(19) United States
(12) Patent Application Publication
(10) Pub. No.: US 2006/0111347 A1
(43) Pub. Date: May 25, 2006

(54) Substituted sulfones and methods of use

(75) Inventors: Benny C. Askew JR., Marshfield, MA (US); Toshihiro Aya, Thousand Oaks, CA (US); Kaustav Biswas, Calabasas, CA (US); Jian J. Chen, Newbury Park, CA (US); Jason Brooks Human, Gasteria, NC (US); Wenyuan Qian, Camarillo, CA (US)

Correspondence Address: AMGEN INC., 1120 VETERANS BOULEVARD, SOUTH SAN FRANCISCO, CA, 94080, US

(73) Assignee: AMGEN INC., THOUSAND OAKS, CA (US)

(21) Appl. No.: 11/243,645

(22) Filed: Oct. 5, 2005

Related U.S. Application Data
(60) Provisional application No. 60/616,685, filed on Oct. 6, 2004.

(51) Int. Cl. A61K 31/5415; A61K 31/47; A61K 31/382; A61K 31/353

(52) U.S. Cl. 514/226.5; 514/311; 514/432; 514/456; 546/161; 549/23; 549/403; 544/49

(57) ABSTRACT

Selected compounds are effective for treatment of pain and diseases, such as inflammation mediated diseases. The invention encompasses novel compounds, analogs, prodrugs and pharmaceutically acceptable derivatives thereof, pharmaceutical compositions and methods for prophylaxis and treatment of diseases and other maladies or conditions involving pain, inflammation, and the like. The subject invention also relates to processes for making such compounds as well as to intermediates useful in such processes.

### CROSS-REFERENCE TO RELATED APPLICATIONS

This application claims the benefit under 35 U.S.C. 119(e) of U.S. Provisional Application Ser. No. 60/616,685, filed on Oct. 6, 2004, the disclosure of which is incorporated herein by reference in its entirety.

### BACKGROUND OF THE INVENTION

1. Field of the Invention

This invention is in the field of pharmaceutical agents and specifically relates to compounds, compositions, uses and methods for treating inflammation-related disorders, including pain.

2. State of the Art

More than two million people in the United States alone are incapacitated by chronic pain on any given day (T. Jessell & D. Kelly, Pain and Analgesia in PRINCIPLES OF NEURAL SCIENCE, third edition (E. Kandel, J. Schwartz, T. Jessell, eds., (1991)). Unfortunately, current treatments for pain are only partially effective, and many cause lifestyle altering, debilitating, and/or dangerous side effects. For example, non-steroidal anti-inflammatory drugs (“NSAIDs”) such as aspirin, ibuprofen, and indomethacin are moderately effective against inflammatory pain but they are also renally toxic, and high doses tend to cause gastrointestinal irritation, ulceration, bleeding, increased cardiovascular risk, and confusion. Patients treated with opioids frequently experience confusion and constipation, and long-term opioid use is associated with tolerance and dependence. Local anesthetics such as lidocaine and mixelitine simultaneously inhibit pain and cause loss of normal sensation. In addition, when used systemically, local anesthetics are associated with adverse cardiovascular effects. Thus, there is currently an unmet need in the treatment of chronic pain.

Pain is a perception based on signals received from the environment and transmitted and interpreted by the nervous system (for review, see M. Millan, Prog. Neurobiol. 57:1-164 (1999)). Noxious stimuli such as heat and touch cause specialized sensory receptors in the skin to send signals to the central nervous system (“CNS”). This process is called nociception, and the peripheral sensory neurons that mediate it are nociceptors. Depending on the strength of the signal from the nociceptor(s) and the abstraction and elaboration of that signal by the CNS, a person may or may not experience a noxious stimulus as painful. When one's perception of pain is properly calibrated to the intensity of the stimulus, pain serves its intended protective function. However, certain types of tissue damage cause a phenomenon, known as hyperalgesia or pronociception, in which relatively innocuous stimuli are perceived as intensely painful because the person's pain thresholds have been lowered. Both inflammation and nerve damage can induce hyperalgesia. Thus, persons afflicted with inflammatory conditions, such as sunburn, osteoarthritis, colitis, carditis, dermatitis, myositis, neuritis, inflammatory bowel disease, collagen vascular diseases (which include rheumatoid arthritis and lupus) and the like, often experience enhanced sensations of pain. Similarly, trauma, surgery, amputation, abscess, causalgia, collagen vascular diseases, demyelinating diseases, trigeminal neuralgia, cancer, chronic alcoholism, stroke, thalamic pain syndrome, diabetes, herpes infections, acquired immune deficiency syndrome (“AIDS”), toxins and chemotherapy cause nerve injuries that result in pain.

As the mechanisms by which nociceptors transduce external signals under normal and hyperalgesic conditions become better understood, processes implicated in hyperalgesia can be targeted to inhibit the lowering of the pain threshold and thereby lessen the amount of pain experienced.

Bradykinin (BK) and the related peptide, kallidin (Lys-BK) mediate the physiological actions of kinins on the cardiovascular and renal systems. However, the active peptides, BK and kallidin, are quickly degraded by peptidases in the plasma and other biological fluids and by those released from a variety of cells, so that the half-life of BK in plasma is reported to be approximately 17 seconds (1). BK and kallidin are rapidly metabolized in the body by carboxypeptidase N, which removes the carboxyterminal arginine residue to generate des-Arg BK or des-Arg kallidin. Des-Arg-kallidin is among the predominant kinins in man and mediates the pathophysiological actions of kinins in man. In addition to being a very potent proinflammatory peptide, des-Arg-BK or des-Arg-kallidin is known to induce vasodilation, vascular permeability, and bronchoconstriction (for review, see Regoli and Barabe, Pharmacological Rev, 32(1), 1-46 (1980)). In addition, des-Arg-BK and des-Arg-kallidin appear to be particularly important mediators of inflammation and inflammatory pain as well as being involved in the maintenance thereof. There is also a considerable body of evidence implicating the overproduction of des-Arg-kallidin in conditions in which pain is a prominent feature such as septic shock, arthritis, angina, and migraine.

The membrane receptors that mediate the pleiotropic actions of kinins are of two distinct classes, designated B1 and B2. Both classes of receptors have been cloned and sequenced from a variety of species, including man (Menke, et al, J. Biol. Chem. 269, 21583-21586 (1994); Hess et al, Biochem. Biophys. Res. Commun. 184, 260-268 (1992)). They are typical G protein coupled receptors having seven putative membrane spanning regions. In various tissues, BK receptors are coupled to every known second messenger. B2 receptors, which have a higher affinity for BK, appear to be the most prevalent form of bradykinin receptor. Essentially all normal physiological responses and many pathophysio-logical responses to bradykinin are mediated by B2 receptors.

B1 receptors, on the other hand, have a higher affinity for des-Arg-BK compared with BK, whereas des-Arg-BK is inactive at B2 receptors. In addition, B1 receptors are not normally expressed in most tissues. Their expression is induced upon injury or tissue damage as well as in certain kinds of chronic inflammation or systemic insult (F. Marceau, et al., Immunopharmacology, 30, 1-26 (1995)). Furthermore, responses mediated by B1 receptors are up-regulated from a null level following administration of bacterial lipopolysaccharide (LPS) or inflammatory cytokines in rabbits, rats, and pigs.

The pain-inducing properties of kinins coupled with the inducible expression of B1 receptors make the B1 receptor an interesting target in the development of anti-inflammatory, antinociceptive, antihyperalgesic and analgesic agents that may be directed specifically at injured tissues with minimal actions in normal tissues.

Certain compounds have been described as bradykinin antagonists. WO 03/07958, published 30 Jan. 2003, describes tetrahydroquinoxalines. Dihydroquinoxalinones are described in a JACS communication.

piperazine-2,3,5-triones are described in Tet. Lett., 40, 7557-7560 (1999). European application 641779, published 8 Mar. 1995, describes 3,6-dioxopiperazines as platelet aggregation inhibitors.

Clearly, there is a need for new, safe and effective treatments for inflammation and pain. Such agents are provided in the present invention.

### DETAILED DESCRIPTION OF THE INVENTION

In one aspect, this invention is directed to a compound of Formula (I):

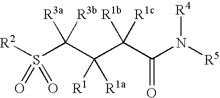

wherein:

R¹ is selected from H, R^{g}, halo, cyano, nitro, —C(═O)R^{b}, —C(═O)OR^{a}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{a}, —S(═O)₂N(R^{a})C(═O)NR^{a}R^{a}, —NR^{a}R^{a}, N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}, benzyl and C₁₋₆alkyl, with the benzyl and C₁₋₆alkyl being substituted by 0, 1, 2, or 3 groups independently selected from R^{g}, cyano, oxo, nitro, —C(═O)R^{b}, —C(═O)OR^{a}, —C(═O)NR^{a}R^{a}, —C(═NR^{a})NR^{a}R^{a}, —OR^{a}, —OC(═O)R^{b}, —OC(═O)NR^{a}R^{a}, OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1, 2, 3, 4, 5 or 6 atoms selected from Br, Cl, F and I;

R^{1a} and R^{1b} are each independently, H, F, Cl, —OH, OCH₃, C₁₋₂alkyl or CF₃;

R^{1c} is H, C₁₋₈alkyl, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}, or C₁₋₆alkyl substituted by 0, 1, 2 or 3 substituents independently selected from C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} or —NR^{a}C₂₋₆alkylOR^{a};

R^{3a} is H, R^{g}, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}, benzyl or C₁₋₆alkyl, with the benzyl and C₁₋₆alkyl being substituted by 0, 1, 2 or 3 substituents independently selected from R^{e}, R^{g}, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alklOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1, 2, 3, 4, 5 or 6 atom selected from Br, Cl, F and I;

R^{3b} is H, F, Cl, OCH₃, C₁₋₂alkyl or CF₃; or

R^{3a} and R^{3b} together are C₂₋₆alkylenyl to form a spiroalkyl that is substituted by 0, 1, 2 or 3 substituents independently selected from R^{e}, R^{g}, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1, 2, 3, 4, 5 or 6 atom selected from Br, Cl, F and I;

R² is a saturated, partially saturated or unsaturated 5-, 6- or 7-membered monocyclic or 6-, 7-, 8-, 9-, 10- or 11-membered bicyclic hydrocarbon ring containing 0, 1, 2, 3 or 4 atoms independently selected from N, O and S, wherein the carbon atoms of the ring are substituted by 0, 1 or 2 oxo groups and the ring is substituted by 0, 1, 2 or 3 substituents selected from R^{e}, R^{g}, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and the ring is additionally substituted by 0, 1, 2, 3, 4 or 5 substituents independently selected from Br, Cl, F and I;

R⁴ is H, phenyl, benzyl or C₁₋₆alkyl, the phenyl, benzyl and C₁₋₆alkyl being substituted by 0, 1, 2 or 3 substituents independently selected from C₁₋₄alkyl, C₁₋₃haloalkyl, —OC₁₋₄alkyl, —NH₂, —NHC₁₋₄alkyl, and —N(C₁₋₄alkyl)C₁₋₄alkyl, and additionally substituted by 0, 1, 2, 3, 4, 5 or 6 atom selected from Br, Cl, F and I;

R⁵ is -(alkylene)n-R where n is 0 or 1 and R is a 5-, 6-, 7-, or 8-membered saturated, partially saturated or unsaturated monocyclic, a saturated, partially saturated or unsaturated 8-, 9-, 10- or 11-membered bicyclic or 12-, 13-, 14- or 15-membered tricyclic hydrocarbon ring containing 0, 1, 2, 3 or 4 atoms selected from N, O and S, wherein the carbon and sulfur atoms of the ring are substituted by 0, 1 or 2 oxo groups and the ring is substituted by R⁶, R⁷, or R⁸ which are independently selected from basic moieties, and additionally substituted by 0, 1, 2 or 3 substituents selected from R⁶, R⁷, and R⁸ which are independently selected from R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and the ring is additionally substituted by 0, 1, 2, 3, 4 or 5 substituents selected from R⁶, R⁷, R⁸, R⁹ and R¹⁰ which are independently selected from Br, Cl, F and I; or

R^{1c} and R⁴ together may additionally be C₂₋₄alkylene substituted by 0, 1 or 2 substituents independently selected from C₁₋₈alkyl, C₁₋₄haloalkyl, halo, oxo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1, 2, 3 or 4 substituents independently selected from Br, Cl, F and I;

R^{a} is independently, at each instance, H or R^{b};

R^{b} is independently, at each instance, phenyl, benzyl or C₁₋₆alkyl, the phenyl, benzyl and C₁₋₆alkyl being substituted by 0, 1, 2 or 3 substituents selected from halo, C₁₋₄alkyl, C₁₋₃haloalkyl, —OC₁₋₄alkyl, —NH₂, —NHC₁₋₄alkyl, —N(C₁₋₄alkyl)C₁₋₄alkyl;

R^{d} is independently, at each instance, C₁₋₈alkyl, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} or —NR^{a}C₂₋₆alkylOR^{a};

R^{e} is independently, at each instance, C₁₋₆alkyl substituted by 0, 1, 2 or 3 substituents independently selected from R^{d} and additionally substituted by 0 or 1 substituents selected from R^{g}; and

R^{g} is independently, at each instance, a saturated, partially saturated or unsaturated 5-, 6- or 7-membered monocyclic or 6-, 7-, 8-, 9-, 10- or 11-membered bicyclic hydrocarbon ring containing 0, 1, 2, 3 or 4 atoms selected from N, O and S, wherein the carbon atoms of the ring are substituted by 0, 1 or 2 oxo groups and the ring is substituted by 0, 1, 2 or 3 substituents selected from C₁₋₈alkyl, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and the ring is additionally substituted by 0, 1, 2, 3, 4 or 5 substituents independently selected from Br, Cl, F and I; or

any pharmaceutically-acceptable salt or hydrate thereof.

Preferably, R¹ is selected from H, R^{g}, cyano, nitro, —C(═O)R^{b}, —C(═O)OR^{a}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}, benzyl and C₁₋₆alkyl, with the benzyl and C₁₋₆alkyl being substituted by 0, 1, 2, or 3 groups independently selected from R^{g}, cyano, oxo, nitro, —C(═O)R^{b}, —C(═O)OR^{a}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1, 2, 3, 4, 5 or 6 atoms selected from Br, Cl, F and I;

R⁵ is a saturated, partially saturated or unsaturated 8-, 9-, 10- or 11-membered bicyclic or 12-, 13-, 14- or 15-membered tricyclic ring hydrocarbon containing 0, 1, 2, 3 or 4 atoms selected from N, O and S, wherein the carbon and sulfur atoms of the ring are substituted by 0, 1 or 2 oxo groups and the ring is substituted by R⁶, R⁷ or R⁸ independently selected from basic moieties, and additionally substituted by 0, 1, 2 or 3 substituents independently selected from R⁶, R⁷ and R⁸which are independently selected from R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and the ring is additionally substituted by 0, 1, 2, 3, 4 or 5 substituents selected from R⁶, R⁷, R⁸, R⁹ and R¹⁰ which are independently selected from Br, Cl, F and I; and

R^{1a} and R^{1b} are each independently, H, F, Cl, OCH₃, C₁₋₂alkyl or CF₃.

In another embodiment, in conjunction with any one of the above and below embodiments, the basic moieties are independently selected from amino, mono-C₁₋₄-alkylamino-C₁₋₄-alkyl, di-C₁₋₄-alkylamino-C₁₋₄-alkyl, mono-C₁₋₄-alkylamino-C₂₋₄-alkenyl, di-C₁₋₄-alkylamino-C₂₋₄-alkenyl, 5-8 membered nitrogen-containing heterocyclyl-C₂₋₄-alkenyl, optionally substituted 5-6 membered nitrogen-containing heterocyclyl and 5-8 membered nitrogen-containing heterocyclyl-C₁₋₄-alkyl.

In another embodiment, in conjunction with any one of the above and below embodiments, the basic moieties are independently selected from amino, aminomethyl, isopropylaminomethyl, t-butylaminomethyl, 2-t-butylaminoethyl, 2-tert-butylamino-1-methylethyl, 1-tert-butylaminoethyl, 1-(tert-butylamino-methyl)-vinyl, 1-(piperidin-1-ylmethyl)-vinyl, N-isobutyl-aminomethyl, N-isobutyl-aminoethyl, (2,2-dimethyl)propylaminomethyl, N-isopropyl-N-ethylaminomethyl, N-isopropyl-N-methylaminomethyl, N-t-butyl-N-methylaminomethyl, N-iso-butyl-N-methylaminomethyl, N-1-butyl-N-ethylaminomethyl, N-isobutyl-N-methylaminomethyl, N-t-butyl-N-isopropylaminomethyl, N,N-di(isopropyl)-aminomethyl, N,N-dimethylaminomethyl, N,N-diethylaminomethyl, N,N-di(1-butyl)-aminomethyl, cyclopropylaminomethyl, cyclopropylaminoethyl, cyclopropylmethyl-aminomethyl, cyclopropylmethylaminoethyl, cyclobutylaminomethyl, cyclobutylaminoethyl, cyclobutylmethylaminomethyl, cyclobutylmethylaminoethyl, 4,5-dihydro-imidazolyl, 1-piperidinylmethyl, 4-fluoropiperidin-1-ylmethyl, 4,4-difluoropiperidin-1-ylmethyl, 3-hydroxypiperidin-1-ylmethyl, 4-hydroxypiperidin-1-ylmethyl, 4-(piperidin-1-yl)-piperidinylmethyl, 4-(dimethylamino)piperidin-1-ylmethyl, 2,6-dimethylpiperidin-1-ylmethyl, 4-morpholinylmethyl, 1-pyrrolidinylmethyl, 2-methylpyrrolidin-1-ylmethyl, 2,5-dimethyl-pyrrolidin-1-ylmethyl, piperazin-1-ylmethyl, azocan-1-ylmethyl, azepan-1-ylmethyl, (7-azabicyclo[2.2.1]hept-7-yl)methyl, (1,3,3-trimethyl-6-azaicyclo[3.2.1]oct-6-yl)methyl, 2-piperidinyl and 4-methylpiperazin-1-ylmethyl.

In another embodiment, in conjunction with any one of the above and below embodiments, R¹ is H.

In another embodiment, in conjunction with any one of the above and below embodiments, R¹ is OR^{a} where R^{a} is hydrogen or C₁₋₆alkyl and R^{1a} is hydrogen.

In another embodiment, in conjunction with any one of the above and below embodiments, R¹ is selected from R^{g}, cyano, nitro, —C(═O)R^{b}, —C(═O)OR^{a}, —C(═O)NR^{a}R^{a}, —C(═NR^{a})NR^{a}R^{a}, —OR^{a}, —OC(═O)R^{b}, —C(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{a}, —S(═O)₂N(R^{a})C(═O)NR^{a}R^{a}, —NR^{a}R^{a}, N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, benzyl and C₁₋₆alkyl, with the benzyl and C₁₋₆alkyl being substituted by 0, 1, 2, or 3 groups independently selected from R^{g}, cyano, oxo, nitro, —C(═O)R^{b}, —C(═O)OR^{a}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1, 2, 3, 4, 5 or 6 atoms selected from Br, Cl, F and I.

In another embodiment, in conjunction with any one of the above and below embodiments, R¹ is R^{g}.

In another embodiment, in conjunction with any one of the above and below embodiments, R¹ is selected from cyano, nitro, —C(═O)R^{b}, —C(═O)OR^{a}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}benzyl and C₁₋₆alkyl, with the benzyl and C₁₋₆alkyl being substituted by 0, 1, 2, or 3 groups independently selected from R^{g}, cyano, oxo, nitro, —C(═O)R^{b}, —C(═O)OR^{a}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1, 2, 3, 4, 5 or 6 atoms selected from Br, Cl, F and I.

In another embodiment, in conjunction with any one of the above and below embodiments, R¹ is —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} or —NR^{a}C₂₋₆alkylOR^{a}.

In another embodiment, in conjunction with any one of the above and below embodiments, R¹ is benzyl and C₁₋₆alkyl, with the benzyl and C₁₋₆alkyl being substituted by 0, 1, 2, or 3 groups independently selected from R^{g}, cyano, oxo, nitro, —C(═O)R^{b}, —C(═O)OR^{a}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1, 2, 3, 4, 5 or 6 atoms selected from Br, Cl, F and I.

In another embodiment, in conjunction with any one of the above and below embodiments, R¹ is C₁₋₆alkyl substituted by 1, 2, or 3 groups independently selected from R^{g}, cyano, oxo, nitro, —C(═O)R^{b}, —C(═O)OR^{a}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1, 2, 3, 4, 5 or 6 atoms selected from Br, Cl, F and I.

In another embodiment, in conjunction with any one of the above and below embodiments, R¹ is C₁₋₆alkyl.

In another embodiment, in conjunction with any one of the above and below embodiments, R¹ is phenyl.

In another embodiment, in conjunction with any one of the above and below embodiments, R^{1a} and R^{1b} are each independently, H, F, Cl, OCH₃, C₁₋₂alkyl or CF₃.

In another embodiment, in conjunction with any one of the above and below embodiments, R^{1a} is H and R^{1b} is F, Cl, OCH₃, C₁₋₂alkyl or CF₃.

In another embodiment, in conjunction with any one of the above and below embodiments, R^{1a} is F, Cl, OCH₃, C₁₋₂alkyl or CF₃ and R^{1b} is H.

In another embodiment, in conjunction with any one of the above and below embodiments, R^{1a} and R^{1b} are each independently, H or F.

In another embodiment, in conjunction with any one of the above and below embodiments, R^{1b} is hydrogen or OCH₃ and R^{1c} is hydrogen.

In another embodiment, in conjunction with any one of the above and below embodiments, R^{1c} is C₁₋₈alkyl, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}, or C₁₋₆alkyl substituted by 0, 1, 2 or 3 substituents selected from C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} or —NR^{a}C₂₋₆alkylOR^{a}.

In another embodiment, in conjunction with any one of the above and below embodiments, R^{1c} is C₁₋₆alkyl substituted by 0, 1, 2 or 3 substituents selected from C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} or —NR^{a}C₂₋₆alkylOR^{a};

In another embodiment, in conjunction with any one of the above and below embodiments, R^{1c} is H or F.

In another embodiment, in conjunction with any one of the above and below embodiments, R² is phenyl or napthyl, both of which are substituted by 0, 1, 2 or 3 substituents selected from R^{e}, R^{g}, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆ alkylNR^{a}R^{a} and —NR^{a}C₂₋₆alkylOR^{a}, and additionally substituted by 0, 1, 2, 3, 4 or 5 substituents independently selected from Br, Cl, F and I.

In another embodiment, in conjunction with any one of the above and below embodiments, R² is phenyl or napthyl, both of which are substituted by 1, 2 or 3 substituents selected from R^{e}, R^{g}, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, OR^{a}, OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆ alkylNR^{a}R^{a} and —NR^{a}C₂₋₆alkylOR^{a}, and additionally substituted by 0, 1, 2, 3, 4 or 5 substituents independently selected from Br, Cl, F and I.

In another embodiment, in conjunction with any one of the above and below embodiments, R² is a saturated, partially saturated or unsaturated 5-, 6- or 7-membered monocyclic or 6-, 7-, 8-, 9-, 10- or 11-membered bicyclic ring containing 1, 2, 3 or 4 atoms selected from N, O and S, wherein the carbon atoms of the ring are substituted by 0, 1 or 2 oxo groups and the ring is substituted by 0, 1, 2 or 3 substituents selected from R^{e}, R^{g}, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and the ring is additionally substituted by 0, 1, 2, 3, 4 or 5 substituents independently selected from Br, Cl, F and I.

In another embodiment, in conjunction with any one of the above and below embodiments, R is an unsaturated 5-, 6- or 7-membered monocyclic ring containing 1, 2 or 3 atoms selected from N, O and S, wherein the carbon atoms of the ring are substituted by 0, 1 or 2 oxo groups and the ring is substituted by 0, 1, 2 or 3 substituents selected from R^{e}, R^{g}, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and the ring is additionally substituted by 0, 1, 2, 3, 4 or 5 substituents independently selected from Br, Cl, F and I.

In another embodiment, in conjunction with any one of the above and below embodiments, R² is selected from 2-naphthyl, 1-naphthyl, phenyl, 3-chlorophenyl, 4-chlorophenyl, 3,5-dichlorophenyl, 3,4-dichlorophenyl, 2,4,6-trichlorophenyl, 3-fluorophenyl, 3-methoxyphenyl, 4-methoxyphenyl, 3-biphenyl, 3-chloro-4-methylphenyl, 4-chloro-3-methylphenyl, 3-trifluoromethylphenyl, 4-trifluoromethylphenyl, 4-trifluoromethoxyphenyl, 3-methylphenyl, 2,1,3-benzoxadiazol-4-yl, thien-2-yl, 3-pyridyl, 8-quinolyl and 5-isoquinolyl.

In another embodiment, in conjunction with any one of the above and below embodiments, R^{3a} is H.

In another embodiment, in conjunction with any one of the above and below embodiments, R^{3a} is R^{g}, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a} benzyl or C₁₋₆alkyl, with the benzyl and C₁₋₆alkyl being substituted by 0, 1, 2 or 3 substituents selected from R^{e}, R^{g}, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1, 2, 3, 4, 5 or 6 atom selected from Br, Cl, F and I.

In another embodiment, in conjunction with any one of the above and below embodiments, R^{3a} is R^{g}.

In another embodiment, in conjunction with any one of the above and below embodiments, R^{3a} is halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} or —NR^{a}C₂₋₆alkylOR^{a}.

In another embodiment, in conjunction with any one of the above and below embodiments, R^{3a} is benzyl or C₁₋₆alkyl, with the benzyl and C₁₋₆alkyl being substituted by 0, 1, 2 or 3 substituents selected from R^{e}, R^{g}, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆ alkylNR^{a}R^{a} and —NR^{a}C₂₋₆alkylOR^{a}, and additionally substituted by 0, 1, 2, 3, 4, 5 or 6 atom selected from Br, Cl, F and I.

In another embodiment, in conjunction with any one of the above and below embodiments, R^{3b} is H.

In another embodiment, in conjunction with any one of the above and below embodiments, R^{3b} is F, Cl, OCH₃, C₁₋₂alkyl or CF₃; or R^{3a} and R^{3b} together are C₂₋₆alkylenyl to form a spiroalkyl that is substituted by 0, 1, 2 or 3 substituents selected from R^{e}, R^{g}, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1, 2, 3, 4, 5 or 6 atom selected from Br, Cl, F and I.

In another embodiment, in conjunction with any one of the above and below embodiments, R⁴ is H.

In another embodiment, in conjunction with any one of the above and below embodiments, R⁴ is phenyl, benzyl or C₁₋₆alkyl, the phenyl, benzyl and C₁₋₆alkyl being substituted by 0, 1, 2 or 3 substituents selected from C₁₋₄alkyl, C₁₋₃haloalkyl, —OC₁₋₄alkyl, —NH₂, —NHC₁₋₄alkyl, and —N(C₁₋₄alkyl)C₁₋₄alkyl, and additionally substituted by 0, 1, 2, 3, 4, 5 or 6 atom selected from Br, Cl, F and I.

In another embodiment, in conjunction with any one of the above and below embodiments, R⁵ is:

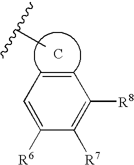

wherein:

the C ring is a saturated or partially saturated 6- or 7-membered ring containing 0, 1 or 2 atoms selected from N, O and S, wherein the carbon and sulfur atoms of the ring are substituted by 0, 1 or 2 oxo groups and the ring is substituted by 0 or 1 substituents selected from R^{g}, C₁₋₈alkyl, C₁₋₄haloalkyl, cyano, nitro, —C(═C))R^{b}, —(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and the ring is additionally substituted by 0, 1, 2 or 3 substituents independently selected from Br, Cl, F and I; and

R⁶, R⁷ and R⁸ are independently selected from H, a basic moiety, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}, Br, Cl, F and I; provided that 1 or 2 of R⁶, R⁷ and R⁸ are a basic moiety.

In another embodiment, in conjunction with any one of the above and below embodiments, R⁶ and R⁸ are H; and wherein R⁷ is selected from amino, aminomethyl, isopropylaminomethyl, 1-butylaminomethyl, 2-t-butylaminoethyl, 2-tert-butylamino-1-methylethyl, 1-tert-butylaminoethyl, 1-(tert-butylamino-methyl)-vinyl, 1-(piperidin-1-ylmethyl)-vinyl, N-isobutyl-aminomethyl, N-isobutyl-aminoethyl, (2,2-dimethyl)propylaminomethyl, N-isopropyl-N-ethylaminomethyl, N-isopropyl-N-methylaminomethyl, N-t-butyl-N-methylaminomethyl, N-iso-butyl-N-methylaminomethyl, N-t-butyl-N-ethylaminomethyl, N-isobutyl-N-methylaminomethyl, N-t-butyl-N-isopropylaminomethyl, N,N-di(isopropyl)aminomethyl, N,N-dimethylaminomethyl, N,N-diethylaminomethyl, N,N-di(t-butyl)-aminomethyl, cyclopropylaminomethyl, cyclopropylaminoethyl, cyclopropylmethylaminomethyl, cyclopropylmethylaminoethyl, cyclobutylaminomethyl, cyclobutylaminoethyl, cyclobutylmethylaminomethyl, cyclobutylmethylaminoethyl, 4,5-dihydro-imidazolyl, 1-piperidinylmethyl, 4-fluoropiperidin-1-ylmethyl, 4,4-difluoropiperidin-1-ylmethyl, 3-hydroxypiperidin-1-ylmethyl, 4-hydroxypiperidin-1-ylmethyl, 4-(piperidin-1-yl)piperidinylmethyl, 4-(dimethylamino)piperidin-1-ylmethyl, 2,6-dimethylpiperidin-1-ylmethyl, 4-morpholinylmethyl, 1-pyrrolidinylmethyl, 2-methylpyrrolidin-1-ylmethyl, 2,5-dimethylpyrrolidin-1-ylmethyl, piperazin-1-ylmethyl, azocan-1-ylmethyl, azepan-1-ylmethyl, (7-azabicyclo[2.2.1]hept-7-yl)methyl, (1,3,3-trimethyl-6-azaicyclo[3.2.1]oct-6-yl)methyl, 2-piperidinyl and 4-methylpiperazin-1-ylmethyl.

In another embodiment, in conjunction with any one of the above and below embodiments, R⁷ and R⁸ are H; and R⁶ is selected from amino, aminomethyl, isopropylaminomethyl, t-butylaminomethyl, 2-t-butylaminoethyl, 2-tert-butylamino-1-methylethyl, 1-tert-butylaminoethyl, 1-(tert-butylamino-methyl)-vinyl, 1-(piperidin-1-ylmethyl)-vinyl, N-isobutyl-aminomethyl, N-isobutyl-aminoethyl, (2,2-dimethyl)propylaminomethyl, N-isopropyl-N-ethylaminomethyl, N-isopropyl-N-methylaminomethyl, N-t-butyl-N-methylaminomethyl, N-iso-butyl-N-methylaminomethyl, N-t-butyl-N-ethylaminomethyl, N-isobutyl-N-methylaminomethyl, N-t-butyl-N-isopropylaminomethyl, N,N-di(isopropyl)-aminomethyl, N,N-dimethylaminomethyl, N,N-diethylaminomethyl, N,N-di(t-butyl)-aminomethyl, cyclopropylaminomethyl, cyclopropylaminoethyl, cyclopropylmethylaminomethyl, cyclopropylmethylaminoethyl, cyclobutylaminomethyl, cyclobutylaminoethyl, cyclobutylmethylaminomethyl, cyclobutylmethylaminoethyl, 4,5-dihydro-imidazolyl, I-piperidinylmethyl, 4-fluoropiperidin-1-ylmethyl, 4,4-difluoropiperidin-1-ylmethyl, 3-hydroxypiperidin-1-ylmethyl, 4-hydroxypiperidin-1-ylmethyl, 4-(piperidin-1-yl)-piperidinylmethyl, 4-(dimethylamino)piperidin-1-ylmethyl, 2,6-dimethylpiperidin-1-ylmethyl, 4-morpholinylmethyl, 1-pyrrolidinylmethyl, 2-methylpyrrolidin-1-ylmethyl, 2,5-dimethylpyrrolidin-1-ylmethyl, piperazin-1-ylmethyl, azocan-1-ylmethyl, azepan-1-ylmethyl, (7-azabicyclo[2.2.1]hept-7-yl)methyl, (1,3,3-trimethyl-6-azaicyclo[3.2.1]oct-6-yl)methyl, 2-piperidinyl and 4-methylpiperazin-1-ylmethyl.

In another embodiment, in conjunction with any one of the above and below embodiments, R⁶ and R⁷ are H; and R⁸ is selected from amino, aminomethyl, isopropylaminomethyl, t-butylaminomethyl, 2-t-butylaminoethyl, 2-tert-butylamino-1-methylethyl, 1-tert-butylaminoethyl, 1-(tert-butylamino-methyl)-vinyl, 1-(piperidin-1-ylmethyl)-vinyl, N-isobutyl-aminomethyl, N-isobutyl-aminoethyl, (2,2-dimethyl)propylaminomethyl, N-isopropyl-N-ethylaminomethyl, N-isopropyl-N-methylaminomethyl, N-t-butyl-N-methylaminomethyl, N-iso-butyl-N-methylaminomethyl, N-t-butyl-N-ethylaminomethyl, N-isobutyl-N-methylaminomethyl, N-t-butyl-N-isopropylaminomethyl, N,N-di(isopropyl)-aminomethyl, N,N-dimethylaminomethyl, N,N-diethylaminomethyl, N,N-di(t-butyl)-aminomethyl, cyclopropylaminomethyl, cyclopropylaminoethyl, cyclopropylmethylaminomethyl, cyclopropylmethylaminoethyl, cyclobutylaminomethyl, cyclobutylaminoethyl, cyclobutylmethylaminomethyl, cyclobutylmethylaminoethyl, 4,5-dihydro-imidazolyl, 1-piperidinylmethyl, 4-fluoropiperidin-1-ylmethyl, 4,4-difluoropiperidin-1-ylmethyl, 3-hydroxypiperidin-1-ylmethyl, 4-hydroxypiperidin-1-ylmethyl, 4-(piperidin-1-yl)piperidinylmethyl, 4-(dimethylamino)piperidin-1-ylmethyl, 2,6-dimethylpiperidin-1-ylmethyl, 4-morpholinylmethyl, 1-pyrrolidinylmethyl, 2-methylpyrrolidin-1-ylmethyl, 2,5-dimethylpyrrolidin-1-ylmethyl, piperazin-1-ylmethyl, azocan-1-ylmethyl, azepan-1-ylmethyl, (7-azabicyclo[2.2.1]-hept-7-yl)methyl, (1,3,3-trimethyl-6-azaicyclo[3.2.1]oct-6-yl)methyl, 2-piperidinyl and 4-methylpiperazin-1-ylmethyl.

In another embodiment, in conjunction with any one of the above and below embodiments, R⁵ is:

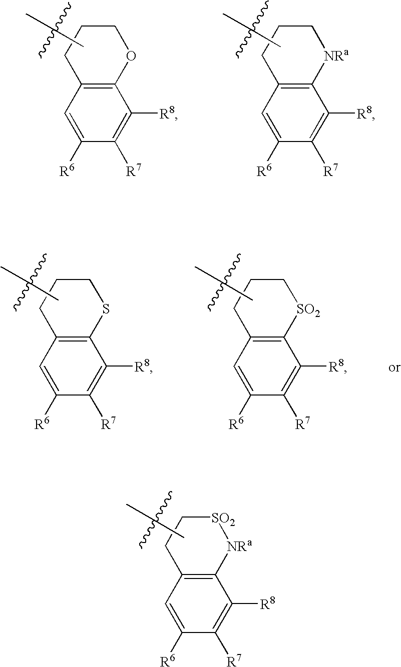

each of which are substituted by 0 or 1 substituents selected from R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR⁸, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1, 2 or 3 substituents independently selected from Br, Cl, F and I.

In another embodiment, in conjunction with any one of the above and below embodiments, R⁵ is:

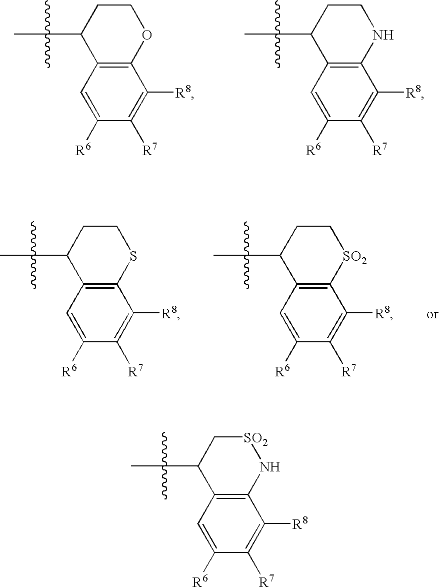

each of which are substituted by 0 or 1 substituents selected from R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1, 2 or 3 substituents independently selected from Br, Cl, F and I.

In another embodiment, in conjunction with any one of the above and below embodiments, R⁵ is:

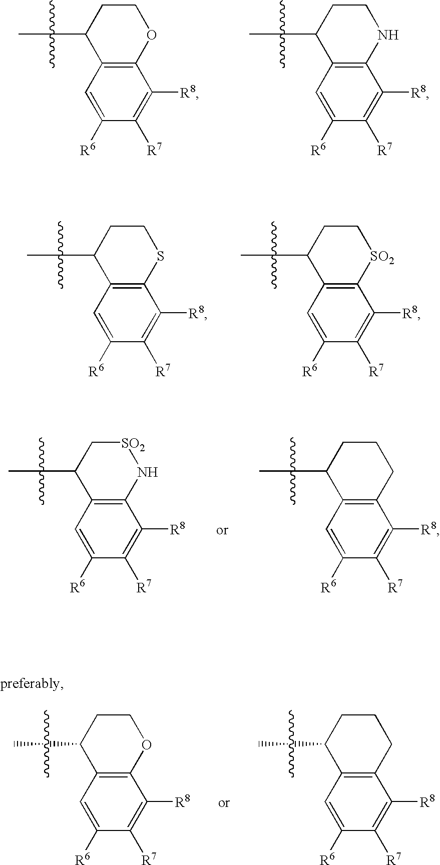

where the part of the above rings that is attached to the nitrogen atom in Formula (I) [i.e., dihydropyranyl, tetrahydropyridinyl, dihydrothiopyranyl, 1,1-dioxodihydrothiopyranyl portion of the ring including the nitrogen ring atom] is substituted by 0 or 1 substituents selected from R^{g}, C₁₋₄alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1, 2 or 3 substituents independently selected from Br, Cl, F and I and R⁶, R⁷ and R⁸ are independently selected from H, a basic moiety, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}, Br, Cl, F and I; provided that 1 or 2 of R⁶, R⁷ and R⁸ are a basic moiety.

In another embodiment, in conjunction with any one of the above and below embodiments, R^{1c} and R⁴ together are C₂₋₄alkylene substituted by 1 or 2 substituents selected from C₁₋₈alkyl, C₁₋₄haloalkyl, halo, oxo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1, 2, 3 or 4 substituents independently selected from Br, Cl, F and I.

In another embodiment, in conjunction with any one of the above and below embodiments, R^{1c} and R⁴ together may additionally be C₂₋₄alkylene.

In another embodiment, in conjunction with any one of the above and below embodiments, R^{3a} is hydrogen, C₁₋₆alkyl, —C(═O)R^{b}, —C(═O)OR^{a}, or —C(═O)NR^{a}R^{a}, preferably hydrogen, methyl, carboxy or methoxycarbonyl;

R^{3b} is hydrogen or C₁₋₂alkyl, preferably hydrogen or methyl; or

R^{3a} and R^{3b} together form C₂₋₃ alkylene, preferably cyclopropylene; and

R⁴ is hydrogen.

Within this embodiment and more preferred groups contained therein, a more preferred group of compounds is that wherein:

R¹ is hydrogen, —OR^{a}, R^{g}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}, —NR^{a}C₂₋₆alkylOR^{a}, and C₁₋₆alkyl being substituted by 0, 1, 2, or 3 groups selected from fluoro or —OR^{a}, preferably hydrogen, hydroxyl, phenyl, methyl, trifluoromethyl, hydroxymethyl, or amino, more preferably hydrogen, hydroxyl or methoxy; and

R^{1a}, R^{1b}, and R^{1c} are independently selected from hydrogen, hydroxyl, methyl, or methoxy, preferably R^{1a} and R^{1b} are hydrogen and R^{1c} is hydrogen, hydroxyl or methoxy.

Within this embodiment and preferred and more preferred groups contained therein, an even more preferred group of compounds is that wherein:

R² is phenyl substituted by 0, 1, 2 or 3 substituents independently selected from C₁₋₄haloalkyl, halo, C₁₋₆alkyl, or —OR^{a} or naphthyl, preferably phenyl, 4-trifluoromethylphenyl, 3-trifluoromethylphenyl, naphth-2-yl, 2,4-dichlorophenyl, 2,3-dichlorophenyl, 2,5-dichlorophenyl, 3,4-dichlorophenyl, 3,5-dichlorophenyl, 2,6-dichlorophenyl, 4-trifluoromethoxyphenyl, 2-fluoro-5-trifluoromethylphenyl, 2-chloro-5-trifluoromethylphenyl, 2-chloro-3-trifluoromethylphenyl, 2-fluoro-5-chlorophenyl, 2-fluoro-3-chlorophenyl, 2,5-dimethyl-4-chlorophenyl, 4-tert-butylphenyl, 3-chlorophenyl, 4-chlorophenyl, 2-chlorophenyl, 2-trifluoromethoxyphenyl, 3-methoxyphenyl, 3-trifluoromethoxyphenyl, 3-methylphenyl, 2-fluoro-4-chlorophenyl, or 2-fluoro-5-chlorophenyl.

Within this embodiment and preferred, more preferred, and even more preferred groups contained therein, a particularly preferred group of compounds is that wherein:

R⁵ is (R)-1,2,3,4-tetrahydronaphth-1-yl or (R)-chroman-4-yl wherein the (R)-1,2,3,4-tetrahydronaphth-1-yl and (R)-chroman-4-yl are substituted at the 6-position and 7-position respectively with mono-C₁₋₄-alkylamino-C₁₋₄-alkyl, di-C₁₋₄-alkylamino-C₁₋₄-alkyl or 5-8 membered nitrogen-containing heterocyclyl-C₁₋₄-alkyl wherein the heterocyclyl is optionally substituted with 1, 2 or 3 groups independently selected from halo, —OH, or (C₁-C₆)alkyl optionally substituted with hydroxyl, preferably piperidin-1-ylmethyl, N-methylpiperazin-1-ylmethyl, 4-methylpiperidin-1-ylmethyl, azepan-1-ylmethyl, 3-methylpiperidin-1-ylmethyl, 3-hydroxymethylpiperidin-1-ylmethyl, 3-hydroxypiperidin-1-ylmethyl, 2,6-dimethylpiperidin-1-ylmethyl, 2-methylpiperidin-1-ylmethyl, or tert-butylaminomethyl.

A family of specific compounds of particular interest consists of compounds and pharmaceutically-acceptable salts thereof as follows:
(3R)-4-(2-naphthalenylsulfonyl)-3-phenyl-N-((4R)-7-(1-piperidinylmethyl)-3,4-dihydro-2H-chromen-4-yl)butanamide;
(3S)-3-(acetylamino)-N-((4R)-6-chloro-7-(((1,1-dimethylethyl)amino)methyl)-3,4-dihydro-2H-chromen-4-yl)-4-((3,4-dichlorophenyl)sulfonyl)butanamide;
(3S)-3-(methyloxy)-4-(2-naphthalenylsulfonyl)-N-((1R)-6-(1-piperidinylmethyl)-1,2,3,4-tetrahydro-1-naphthalenyl)butanamide;
(3S)-3-amino-N-((4R)-6-chloro-7-(((1,1-dimethylethyl)amino)methyl)-3,4-dihydro-2H-chromen-4-yl)-4-((3,4-dichlorophenyl)sulfonyl)butanamide;
(3S)-3-hydroxy-4-(2-naphthalenylsulfonyl)-N-((1R)-6-(1-(1-piperidinylmethyl)-ethenyl)-1,2,3,4-tetrahydro-1-naphthalenyl)butanamide;
(3S)-3-hydroxy-4-(2-naphthalenylsulfonyl)-N-((1R)-6-(1-piperidinylmethyl)-1,2,3,4-tetrahydro-1-naphthalenyl)butanamide;
(3S)-3-hydroxy-N-((1R)-6-(((2-methylpropyl)amino)methyl)-1,2,3,4-tetrahydro-1-naphthalenyl)-4-(2-naphthalenylsulfonyl)butanamide;
(3S)-3-hydroxy-N-((1R)-6-((4-methyl-1-piperazinyl)methyl)-1,2,3,4-tetrahydro-1-naphthalenyl)-4-(2-naphthalenylsulfonyl)butanamide;
(3S)-3-hydroxy-N-((1R)-6-(hydroxymethyl)-1,2,3,4-tetrahydro-1-naphthalenyl)-4-(2-naphthalenylsulfonyl)butanamide;
(3S)-3-methyl-4-(2-naphthalenylsulfonyl)-N-((1R)-6-(1-piperidinylmethyl)-1,2,3,4-tetrahydro-1-naphthalenyl)butanamide;
(3S)-3-phenyl-N-((1R)-5-(1-piperidinylmethyl)-2,3-dihydro-1H-inden-1-yl)-4-((3-(trifluoromethyl)phenyl)sulfonyl)butanamide;
(3S)-4-((3,4-dichlorophenyl)sulfonyl)-3-hydroxy-N-((1R)-6-(1-piperidinylmethyl)-1,2,3,4-tetrahydro-1-naphthalenyl)butanamide;
(3S)-4-((4-(1,1-dimethylethyl)phenyl)sulfonyl)-3-hydroxy-N-((1R)-6-(1-piperidinylmethyl)-1,2,3,4-tetrahydro-1-naphthalenyl)butanamide;
(3S)-4-(2-naphthalenylsulfonyl)-3-phenyl-N-((1R)-5-(1-piperidinylmethyl)-2,3-dihydro-1H-inden-1-yl)butanamide;
(3S)-N-((1R)-6-(((1,1-dimethylethyl)amino)methyl)-1,2,3,4-tetrahydro-1-naphthalenyl)-3-phenyl-4-((3-(trifluoromethyl)phenyl)sulfonyl)butanamide;
(3S)-N-((1R)-6-((4-fluoro-1-piperidinyl)methyl)-1,2,3,4-tetrahydro-1-naphthalenyl)-3-hydroxy-4-(2-naphthalenylsulfonyl)butanamide;
(3S)-N-((1R)-6-((cyclopentylamino)methyl)-1,2,3,4-tetrahydro-1-naphthalenyl)-3-hydroxy-4-(2-naphthalenylsulfonyl)butanamide;
(3S)-N-((4R)-6-chloro-7-(((1,1-dimethylethyl)amino)methyl)-3,4-dihydro-2H-chromen-4-yl)-4-((3,4-dichlorophenyl)sulfonyl)-3-hydroxybutanamide;
(R)-2-hydroxy-4-(naphthalen-2-ylsulfonyl)-N-((R)-6-(piperidin-1-ylmethyl)-1,2,3,4-tetrahydronaphthalen-1-yl)butanamide;
1,1-dimethylethyl (1S)-3-(((4R)-6-chloro-7-(((1,1-dimethylethyl)amino)methyl)-3,4-dihydro-2H-chromen-4-yl)amino)-1-(((3,4-dichlorophenyl)sulfonyl)methyl)-3-oxopropylcarbamate;
4-(2-naphthalenylsulfonyl)-N-((1R)-5-(1-piperidinylmethyl)-2,3-dihydro-1H-inden-1-yl)butanamide;
(2R)-2-hydroxy-4-(2-naphthalenyl-sulfonyl)-N-((1R)-6-(1-piperidinylmethyl)-1,2,3,4-tetrahydro-1-naphthalenyl)-butanamide;
(4R,5S)-2,2-dimethyl-5-((naphthalen-2-yl-sulfonyl)methyl)-N-((R)-6-(piperidin-1-yl-methyl)-1,2,3,4-tetrahydronaphthalen-1-yl)-1,3-dioxolane-4-carboxamide;
(2R,3S)-2,3-dihydroxy-4-(naphthalen-2-ylsulfonyl)-N-((R)-6-(piperidin-1-ylmethyl)-1,2,3,4-tetrahydronaphthalen-1-yl)-butanamide;
(S)-4-(naphthalen-2-ylsulfonyl)-3-phenyl-N-((R)-6-(piperidin-1-ylmethyl)-1,2,3,4-tetrahydronaphthalen-1-yl)butanamide;
(R)-4-(naphthalen-2-ylsulfonyl)-3-phenyl-N-((R)-6-(piperidin-1-ylmethyl)-1,2,3,4-tetrahydronaphthalen-1-yl)butanamide;
4-(2-naphthalenylsulfonyl)-N-((1R)-6-(1-piperidinylmethyl)-1,2,3,4-tetrahydro-1-naphthalenyl)butanamide;
(R)-3-(hydroxymethyl)-4-(naphthalen-2-ylsulfonyl)-N-((R)-6-(piperidin-1-ylmethyl)-1,2,3,4-tetrahydronaphthalen-1-yl)-butanamide;
(S)-3-(hydroxymethyl)-4-(naphthalen-2-ylsulfonyl)-N-((R)-6-(piperidin-1-ylmethyl)-1,2,3,4-tetrahydronaphthalen-1-yl)-butanamide;
(R)-4-(naphthalen-2-ylsulfonyl)-3-phenyl-N-(4-(4-(pyridin-4-yl)piperazin-1-yl)-phenyl)butanamide;
(S)-4-(naphthalen-2-ylsulfonyl)-3-phenyl-N-(4-(4-(pyridin-4-yl)piperazin-1-yl)phenyl)-butanamide;
(S)-N-(4-(4,5-dihydro-1H-imidazol-2-yl)phenethyl)-4-(naphthalen-2-ylsulfonyl)-3-phenylbutanamide;
(R)-N-(4-(4,5-dihydro-1H-imidazol-2-yl)-phenethyl)-4-(naphthalen-2-ylsulfonyl)-3-phenylbutanamide;
(2R,3S)-N-((R)-6-chloro-7-(piperidin-1-yl-methyl)chroman-4-yl)-2,3-dihydroxy-4-(naphthalen-2-ylsulfonyl)butanamide;
(2R,3S)-2,3-dihydroxy-N-((R)-6-((4-methyl-piperidin-1-yl)methyl)-1,2,3,4-tetrahydronaphthalen-1-yl)-4-(naphthalen-2-ylsulfonyl)butanamide;
(2R,3S)-4-(3-chlorophenylsulfonyl)-2,3-dihydroxy-N-((R)-6-(piperidin-1-ylmethyl)-1,2,3,4-tetrahydronaphthalen-1-yl)-butanamide;
(R)-2-methoxy-4-(naphthalen-2-ylsulfonyl)-N-((R)-6-(piperidin-1-ylmethyl)-1,2,3,4-tetrahydronaphthalen-1-yl)butanamide;
(2R,3S)-4-(3,4-dichlorophenylsulfonyl)-2,3-dihydroxy-N-((R)-6-(piperidin-1-yl-methyl)-1,2,3,4-tetrahydronaphthalen-1-yl)-butanamide;
(R)-2-hydroxy-3,3-dimethyl-4-(naphthalen-2-ylsulfonyl)-N-((R)-6-(piperidin-1-yl-methyl)-1,2,3,4-tetrahydronaphthalen-1-yl)butanamide;
(2R,3S)-2,3-dihydroxy-N-((R)-6-(piperidin-1-ylmethyl)-1,2,3,4-tetrahydronaphthalen-1-yl)-4-tosylbutanamide;
(2R,3S)-2,3-dihydroxy-N-((R)-6-(piperidin-1-ylmethyl)-1,2,3,4-tetrahydronaphthalen-1-yl)-4-tosylbutanamide;
(S)-2-hydroxy-4-(naphthalen-2-ylsulfonyl)-N-((R)-6-(piperidin-1-ylmethyl)-1,2,3,4-tetrahydronaphthalen-1-yl)butanamide;
(2R,3S)-4-(4-tert-butylphenylsulfonyl)-2,3-dihydroxy-N-((R)-6-(piperidin-1-ylmethyl)-1,2,3,4-tetrahydronaphthalen-1-yl)butanamide;
(2S,3S)-2,3-dihydroxy-3-(1-(naphthalen-2-ylsulfonyl)cyclopropyl)-N-((R)-6-(piperidin-1-ylmethyl)-1,2,3,4-tetrahydronaphthalen-1-yl)propanamide;
(2R,3R)-2,3-dihydroxy-3-(1-(naphthalen-2-ylsulfonyl)cyclopropyl)-N-((R)-6-(piperidin-1-ylmethyl)-1,2,3,4-tetrahydronaphthalen-1-yl)propanamide;
(2R,3S)-N-((R)-6-((2,6-dimethylpiperidin-1-yl)methyl)-1,2,3,4-tetrahydronaphthalen-1-yl)-2,3-dihydroxy-4-(naphthalen-2-yl-sulfonyl)butanamide;
(2R,3S)-2,3-dihydroxy-N-((R)-6-(((R)-2-methylpiperidin-1-yl)methyl)-1,2,3,4-tetrahydronaphthalen-1-yl)-4-(naphthalen-2-ylsulfonyl)butanamide;
(2R,3S)-2,3-dihydroxy-N-((R)-6-(((S)-2-methylpiperidin-1-yl)methyl)-1,2,3,4-tetrahydronaphthalen-1-yl)-4-(naphthalen-2-ylsulfonyl)butanamide;
(S)-3-hydroxy-3-(1-(naphthalen-2-yl-sulfonyl)cyclopropyl)-N-((R)-6-(piperidin-1-yl-methyl)-1,2,3,4-tetrahydronaphthalen-1-yl)propanamide;
(R)-3-hydroxy-3-(1-(naphthalen-2-yl-sulfonyl)cyclopropyl)-N-((R)-6-(piperidin-1-yl-methyl)-1,2,3,4-tetrahydronaphthalen-1-yl)propanamide;
(2R,3S)-2-hydroxy-3-methyl-N-((R)-6-(piperidin-1-ylmethyl)-1,2,3,4-tetrahydronaphthalen-1-yl)-4-(3-(trifluoromethyl)-phenylsulfonyl)butanamide;
(2S,3R)-2,3-dihydroxy-N-((R)-6-(piperidin-1-ylmethyl)-1,2,3,4-tetrahydronaphthalen-1-yl)-4-(3-(trifluoromethyl)phenyl-sulfonyl)butanamide;
(2R,3S)-N-((R)-6-((tert-butylamino)methyl)-1,2,3,4-tetrahydronaphthalen-1-yl)-2,3-dihydroxy-4-(3-(trifluoromethyl)-phenylsulfonyl)butanamide;
(R)-3-(1-(naphthalen-2-ylsulfonyl)-cyclopropyl)-N-(6-(piperidin-1-ylmethyl)-1,2,3,4-tetrahydronaphthalen-1-yl)propanamide;
(2R,3S)-2,3-dihydroxy-N-((R)-6-(piperidin-1-ylmethyl)-1,2,3,4-tetrahydronaphthalen-1-yl)-4-(4-(trifluoromethyl)phenyl-sulfonyl)butanamide;
(2R,3S)-4-(2,3-dichlorophenylsulfonyl)-2,3-dihydroxy-N-((R)-6-(piperidin-1-yl-methyl)-1,2,3,4-tetrahydronaphthalen-1-yl)-butanamide;
(2R,3S)-2,3-dihydroxy-N-((R)-6-((4-methylpiperidin-1-yl)methyl)-1,2,3,4-tetrahydronaphthalen-1-yl)-4-(3-(trifluoromethyl)phenylsulfonyl)butanamide;
(2R,3S)-2,3-dihydroxy-N-((R)-6-(piperidin-1-ylmethyl)-1,2,3,4-tetrahydronaphthalen-1-yl)-4-(3-(trifluoromethyl)phenyl-sulfonyl)butanamide;
(2R,3S)-2,3-dihydroxy-N-((1R)-6-((1R/S)-1-(1-piperidinyl)ethyl)-1,2,3,4-tetrahydro-1-naphthalenyl)-4-((3-(trifluoromethyl)-phenyl)-sulfonyl)butanamide;
(2R,3S)-2,3-dihydroxy-4-(phenylsulfonyl)-N-((R)-6-(piperidin-1-ylmethyl)-1,2,3,4-tetrahydronaphthalen-1-yl)butanamide;
(2R,3S)-2,3-dihydroxy-N-((R)-6-((R)-1-(piperidin-1-yl)ethyl)-1,2,3,4-tetrahydronaphthalen-1-yl)-4-(3-(trifluoromethyl)-phenylsulfonyl)butanamide;
(2R,3S)-2,3-dihydroxy-N-((R)-6-((S)-1-(piperidin-1-yl)ethyl)-1,2,3,4-tetrahydronaphthalen-1-yl)-4-(3-(trifluoromethyl)-phenylsulfonyl)butanamide;
(2R,3S)-2,3-dihydroxy-N-((R)-6-(piperidin-1-ylmethyl)-1,2,3,4-tetrahydronaphthalen-1-yl)-4-(3-(trifluoromethoxy)phenyl-sulfonyl)butanamide;
(2R,3R)-2,3-dihydroxy-N-((R)-6-(piperidin-1-ylmethyl)-1,2,3,4-tetrahydronaphthalen-1-yl)-4-(3-(trifluoromethyl)phenyl-sulfonyl)butanamide;
(2S,3S)-2,3-dihydroxy-N-((1R)-6-(1-piperidinylmethyl)-1,2,3,4-tetrahydro-1-naphthalenyl)-4-((3-(trifluoromethyl)-phenyl)sulfonyl)butanamide;
(2S,3S)-2,3-dihydroxy-N-((R)-6-(piperidin-1-ylmethyl)-1,2,3,4-tetrahydronaphthalen-1-yl)-4-(3-(trifluoromethyl)phenylsulfonyl)-butanamide;
(R)-methyl 2-(naphthalen-2-ylsulfonyl)-5-oxo-5-((R)-6-(piperidin-1-ylmethyl)-1,2,3,4-tetrahydronaphthalen-1-ylamino)pentanoate;
(S)-methyl 2-(naphthalen-2-ylsulfonyl)-5-oxo-5-((R)-6-(piperidin-1-ylmethyl)-1,2,3,4-tetrahydronaphthalen-1-ylamino)pentanoate;
(S)-2-(naphthalen-2-ylsulfonyl)-5-oxo-5-((R)-6-(piperidin-1-ylmethyl)-1,2,3,4-tetrahydronaphthalen-1-ylamino)pentanoic acid;
(R)-2-(naphthalen-2-ylsulfonyl)-5-oxo-5-((R)-6-(piperidin-1-ylmethyl)-1,2,3,4-tetrahydronaphthalen-1-ylamino)pentanoic acid;
1-(((5R)-5-(((2S,3R)-2,3-dihydroxy-4-((3-(trifluoromethyl)phenyl)sulfonyl)butanoyl)-amino)-5,6,7,8-tetrahydro-2-naphthalenyl)methyl)-1-methylpiperidinium;
(2R,3S)-2,3-dihydroxy-N-((R)-7-(piperidin-1-ylmethyl)chroman-4-yl)-4-(3-(trifluoromethyl)phenylsulfonyl)-butanamide;
(2R,3S)-N-((R)-6-((R)-1-(tert-butylamino)ethyl)-1,2,3,4-tetrahydronaphthalen-1-yl)-2,3-dihydroxy-4-(3-(trifluoromethyl)phenyl-sulfonyl)butanamide;
(2R,3S)-N-((R)-6-((S)-1-(tert-butyl-amino)ethyl)-1,2,3,4-tetrahydro-naphthalen-1-yl)-2,3-dihydroxy-4-(3-(trifluoromethyl)phenylsulfonyl)butanamide;
(S)-3-hydroxy-N-((R)-6-(piperidin-1-ylmethyl)-1,2,3,4-tetrahydronaphthalen-1-yl)-4-(3-(trifluoromethyl)phenyl-sulfonyl)butanamide;
(4R,5S)-2,2-dimethyl-N-((1R)-6-(1-piperidinylmethyl)-1,2,3,4-tetrahydro-1-naphthalenyl)-5-(((2-(trifluoromethyl)-phenyl)sulfonyl)methyl)-1,3-dioxolane-4-carboxamide;
(2R,3S)-2,3-dihydroxy-N-((R)-6-(piperidin-1-ylmethyl)-1,2,3,4-tetrahydronaphthalen-1-yl)-4-(2-(trifluoromethyl)phenylsulfonyl)-butanamide;
(S)-4-(naphthalen-2-ylsulfonyl)-N-((R)-6-(piperidin-1-ylmethyl)-1,2,3,4-tetrahydronaphthalen-1-yl)pentanamide;
(R)-4-(naphthalen-2-ylsulfonyl)-N-((R)-6-(piperidin-1-ylmethyl)-1,2,3,4-tetrahydronaphthalen-1-yl)pentanamide;
(R)-2-hydroxy-N-((R)-6-(piperidin-1-ylmethyl)-1,2,3,4-tetrahydronaphthalen-1-yl)-4-(3-(trifluoromethyl)phenylsulfonyl)-butanamide;
(S)-2-hydroxy-N-((R)-6-(piperidin-1-ylmethyl)-1,2,3,4-tetrahydronaphthalen-1-yl)-4-(3-(trifluoromethyl)phenylsulfonyl)-butanamide;
(R)-4-methyl-4-(naphthalen-2-ylsulfonyl)-N-(6-(piperidin-1-ylmethyl)-1,2,3,4-tetrahydronaphthalen-1-yl)pentanamide;
(R)-4-(2,3-dihydrobenzo[b][1,4]dioxin-6-ylsulfonyl)-2-hydroxy-N-((R)-6-(piperidin-1-ylmethyl)-1,2,3,4-tetrahydronaphthalen-1-yl)butanamide;
3-hydroxy-N-(6-(piperidin-1-ylmethyl)-1,2,3,4-tetrahydronaphthalen-1-yl)-4-(2-(trifluoromethoxy)phenylsulfonyl)butanamide;
3-hydroxy-N-(6-(piperidin-1-ylmethyl)-1,2,3,4-tetrahydronaphthalen-1-yl)-4-(4-(trifluoromethoxy)phenylsulfonyl)butanamide;
(R)-2,2-dimethyl-N-(6-(piperidin-1-ylmethyl)-1,2,3,4-tetrahydronaphthalen-1-yl)-4-(3-(trifluoromethyl)phenylsulfonyl)-butanamide;
(R)-N-(6-(piperidin-1-ylmethyl)-1,2,3,4-tetrahydronaphthalen-1-yl)-4-(3-(trifluoromethyl)phenylsulfonyl)butanamide;
(S)-3-hydroxy-4-methyl-N-((R)-6-(piperidin-1-ylmethyl)-1,2,3,4-tetrahydronaphthalen-1-yl)-4-(3-(trifluoromethyl)-phenyl-sulfonyl)pentanamide;
(R)-3-hydroxy-4-methyl-N-((R)-6-(piperidin-1-ylmethyl)-1,2,3,4-tetrahydronaphthalen-1-yl)-4-(3-(trifluoro-methyl)phenylsulfonyl)pentanamide.

In a second aspect, this invention is directed to a pharmaceutical composition comprising a compound of Formula (I) or any pharmaceutically-acceptable salt or hydrate thereof and a pharmaceutically acceptable excipient.

In a third aspect, this invention is directed to a method of treating a disease in a patient mediated by the B1 receptor comprising administering to the patient a pharmaceutical composition comprising a therapeutically effective amount of a compound of Formula (I) or

any pharmaceutically-acceptable salt or hydrate thereof and a pharmaceutically acceptable excipient. Specifically, the compounds of the present invention are useful in the treatment of a disorder such as acute pain, dental pain, back pain, lower back pain, pain from trauma, surgical pain, pain resulting from amputation or abscess, causalgia, fibromyalgia, demyelinating diseases, trigeminal neuralgia, cancer, chronic alcoholism, stroke, thalamic pain syndrome, diabetes, acquired immune deficiency syndrome (“AIDS”), toxins and chemotherapy, general headache, migraine, cluster headache, mixed-vascular and non-vascular syndromes, tension headache, general inflammation, arthritis, rheumatic diseases, lupus, osteoarthritis, inflammatory bowel disorders, inflammatory eye disorders, inflammatory or unstable bladder disorders, psoriasis, skin complaints with inflammatory components, sunburn, carditis, dermatitis, myositis, neuritis, collagen vascular diseases, chronic inflammatory conditions, inflammatory pain and associated hyperalgesia and allodynia, neuropathic pain and associated hyperalgesia and allodynia, diabetic neuropathy pain, sympathetically maintained pain, deafferentation syndromes, asthma, vasomotor or allergic rhinitis, epithelial tissue damage or dysfunction, herpes simplex, post-herpetic neuralgia, disturbances of visceral motility at respiratory, genitourinary, gastrointestinal or vascular regions, wounds, burns, allergic skin reactions, pruritis, vitiligo, general gastrointestinal disorders, colitis, inflammatory bowel disease, gastric ulceration, duodenal ulcers, thalamic pain syndrome, diabetes, toxins and chemotherapy, septic shock, and bronchial disorders.

The invention also provides for the use of the compounds of the present invention for the prevention or for the treatment of a disorder such as acute pain, dental pain, back pain, lower back pain, pain from trauma, surgical pain, pain resulting from amputation or abscess, causalgia, fibromyalgia, demyelinating diseases, trigeminal neuralgia, cancer, chronic alcoholism, stroke, thalamic pain syndrome, diabetes, acquired immune deficiency syndrome (“AIDS”), toxins and chemotherapy, general headache, migraine, cluster headache, mixed-vascular and non-vascular syndromes, tension headache, general inflammation, arthritis, rheumatic diseases, lupus, osteoarthritis, inflammatory bowel disorders, inflammatory eye disorders, inflammatory or unstable bladder disorders, psoriasis, skin complaints with inflammatory components, sunburn, carditis, dermatitis, myositis, neuritis, collagen vascular diseases, chronic inflammatory conditions, inflammatory pain and associated hyperalgesia and allodynia, neuropathic pain and associated hyperalgesia and allodynia, diabetic neuropathy pain, sympathetically maintained pain, deafferentation syndromes, asthma, vasomotor or allergic rhinitis, epithelial tissue damage or dysfunction, herpes simplex, post-herpetic neuralgia, disturbances of visceral motility at respiratory, genitourinary, gastrointestinal or vascular regions, wounds, burns, allergic skin reactions, pruritis, vitiligo, general gastrointestinal disorders, colitis, inflammatory bowel disease, gastric ulceration, duodenal ulcers, thalamic pain syndrome, diabetes, toxins and chemotherapy, septic shock, and bronchial disorders.

In a fourth aspect, this invention is directed to the use of one or more of the compounds of the present invention in the manufacture of a medicament. Preferably, the medicament is useful in the treatment of a disorder such as acute pain, dental pain, back pain, lower back pain, pain from trauma, surgical pain, pain resulting from amputation or abscess, causalgia, fibromyalgia, demyelinating diseases, trigeminal neuralgia, cancer, chronic alcoholism, stroke, thalamic pain syndrome, diabetes, acquired immune deficiency syndrome (“AIDS”), toxins and chemotherapy, general headache, migraine, cluster headache, mixed-vascular and non-vascular syndromes, tension headache, general inflammation, arthritis, rheumatic diseases, lupus, osteoarthritis, inflammatory bowel disorders, inflammatory eye disorders, inflammatory or unstable bladder disorders, psoriasis, skin complaints with inflammatory components, sunburn, carditis, dermatitis, myositis, neuritis, collagen vascular diseases, chronic inflammatory conditions, inflammatory pain and associated hyperalgesia and allodynia, neuropathic pain and associated hyperalgesia and allodynia, diabetic neuropathy pain, sympathetically maintained pain, deafferentation syndromes, asthma, vasomotor or allergic rhinitis, epithelial tissue damage or dysfunction, herpes simplex, post-herpetic neuralgia, disturbances of visceral motility at respiratory, genitourinary, gastrointestinal or vascular regions, wounds, burns, allergic skin reactions, pruritis, vitiligo, general gastrointestinal disorders, colitis, inflammatory bowel disease, gastric ulceration, duodenal ulcers, thalamic pain syndrome, diabetes, toxins and chemotherapy, septic shock, and bronchial disorders.

The compounds of this invention may also act as inhibitors of other receptors or kinases, and thus be effective in the treatment of diseases associated with other protein kinases.

Besides being useful for human treatment, these compounds are also useful for veterinary treatment of companion animals, exotic animals and farm animals, including mammals, rodents, and the like. More preferred animals include horses, dogs, and cats.

Definitions:

Unless otherwise stated, the following terms used in the specification and claims have the meanings given below:

The term “C_{α-β}alkyl” means an alkyl group having a minimum of α and a maximum of β carbon atoms in a branched, cyclical or linear relationship or any combination of the three, wherein α and β represent integers as indicated in this Application. The alkyl groups described in this section may also contain one or two double or triple bonds. When the alkyl group has a double bond it is also referred to herein as alkenyl. When the alkyl group has a triple bond it is also referred to herein as alkynyl. Examples of C₁₋₆alkyl include, but are not limited to, the following: 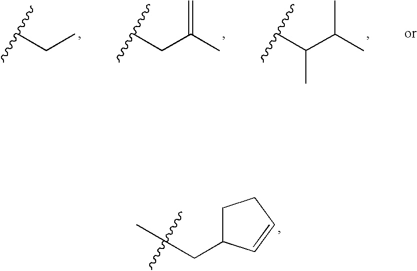
methyl, and the like.

The term “alkylene” or “alkylenyl” means a divalent hydrocarbon radical of one to ten carbon atoms, preferably from two to six carbon atoms unless otherwise stated e.g, methylene, ethylene, propylene, and the like.

The term “alkylamino” denotes amino groups which have been substituted with one or two alkyl radicals, including terms “N-alkylamino” and “N,N-dialkylamino”. More preferred alkylamino radicals are “lower alkylamino” radicals having one or two alkyl radicals of one to six carbon atoms, attached to a nitrogen atom. Even more preferred are lower alkylamino radicals having one to three carbon atoms. Suitable “alkylamino” may be mono or dialkylamino such as N-methylamino, N-ethylamino, N,N-dimethylamino, N,N-diethylamino and the like.

The terms “N-aralkyl-N-alkylamino” and “N-alkyl-N-arylamino” denote amino groups which have been substituted with one aralkyl and one alkyl radical, or one aryl and one alkyl radical, respectively, to an amino group.

The term “aminoalkyl” embraces linear or branched alkyl radicals having one to ten carbon atoms any one of which may be substituted with one or more amino radicals. More preferred aminoalkyl radicals are “lower aminoalkyl” radicals having one to six carbon atoms and one or more amino radicals. Examples of such radicals include aminomethyl, aminoethyl, aminopropyl, aminobutyl and aminohexyl. Even more preferred are lower aminoalkyl radicals having one to three carbon atoms.

The term “alkylaminoalkyl” embraces aminoalkyl radicals having the nitrogen atom independently substituted with an alkyl radical. More preferred alkylaminoalkyl radicals are “lower alkylaminoalkyl” radicals having alkyl radicals of one to six carbon atoms. Even more preferred are lower alkylaminoalkyl radicals having alkyl radicals of one to three carbon atoms. Suitable alkylaminoalkyl radicals may be mono or dialkyl substituted, such as N-methylaminomethyl, N,N-dimethyl-aminoethyl, N,N-diethylaminomethyl and the like.

The term “aminoalkenyl” embraces linear or branched alkenyl radicals having two to ten carbon atoms any one of which may be substituted with one or more amino radicals. More preferred aminoalkenyl radicals are “lower aminoalkenyl” radicals having two to six carbon atoms and one or more amino radicals. Examples of such radicals include aminoethenyl, aminopropenyl, aminobutenyl and aminohexenyl. Even more preferred are lower aminoalkenyl radicals having two or three carbon atoms.

The term “alkylaminoalkenyl” embraces aminoalkenyl radicals having the nitrogen atom independently substituted with an alkyl radical. More preferred alkylaminoalkenyl radicals are “lower alkylaminoalkenyl” radicals having alkyl radicals of one to six carbon atoms. Even more preferred are lower alkylaminoalkenyl radicals having alkyl radicals of one to three carbon atoms. Suitable alkylaminoalkenyl radicals may be mono or dialkyl substituted, such as N-methylaminovinyl, N,N-dimethyl-aminovinyl, N,N-diethylaminovinyl, and the like.

The term “alkoxy” embrace linear or branched oxy-containing radicals (—OR) each having alkyl portions of one to ten carbon atoms. More preferred alkoxy radicals are “lower alkoxy” radicals having one to six carbon atoms. Examples of such radicals include methoxy, ethoxy, propoxy, butoxy and tert-butoxy. Even more preferred are lower alkoxy radicals having one to three carbon atoms. The “alkoxy” radicals may be further substituted with one or more halo atoms, such as fluoro, chloro or bromo, to provide “haloalkoxy” radicals. Even more preferred are lower haloalkoxy radicals having one to three carbon atoms. Examples of such radicals include fluoromethoxy, chloromethoxy, trifluoromethoxy, trifluoroethoxy, fluoroethoxy, and fluoropropoxy.

The term “alkoxyalkyl” embraces linear or branched alkyl radicals having one to about ten carbon atoms any one of which may be substituted with one or more alkoxyl radicals. More preferred alkoxyalkyl radicals are “lower alkoxyalkyl” radicals respectively having one to six carbon atoms. Examples of such radicals include methoxymethyl, methoxyethyl, and the like. Even more preferred are lower alkoxyalkyl radicals respectively having one to three carbon atoms alkyl radicals.

The term “aminoalkoxy” embraces alkoxy radicals substituted with an amino radical. More preferred aminoalkoxy radicals are “lower aminoalkoxy” radicals having alkoxy radicals of one to six carbon atoms. Suitable aminoalkoxy radicals may be aminoethoxy, aminomethoxy, aminopropoxy and the like.

The term “alkylaminoalkoxy” embraces alkoxy radicals substituted with alkylamino radicals. More preferred alkylaminoalkoxy radicals are “lower alkylaminoalkoxy” radicals having alkoxy radicals of one to six carbon atoms. Even more preferred are lower alkylaminoalkoxy radicals having alkyl radicals of one to three carbon atoms. Suitable alkylaminoalkoxy radicals may be mono or dialkyl substituted, such as N-methylaminoethoxy, N,N-dimethylaminoethoxy, N,N-diethylaminoethoxy and the like.

The term “alkylaminoalkoxyalkoxy” embraces alkoxy radicals substituted with alkylaminoalkoxy radicals as defined above. More preferred alkylaminoalkoxyalkoxy radicals are “lower alkylaminoalkoxyalkoxy” radicals independently having alkoxy radicals of one to six carbon atoms. Even more preferred are lower alkylaminoalkoxyalkoxy radicals having alkyl radicals of one to three carbon atoms. Suitable alkylaminoalkoxyalkoxy radicals may be mono or dialkyl substituted, such as N-methylaminoethoxymethoxy, N,N-dimethylaminoethoxymethoxy, N,N-diethylaminomethoxymethoxy, and the like.

The term “aryl”, alone or in combination, means a carbocyclic aromatic system containing one or two rings wherein such rings may be attached together in a pendent manner or may be fused. The term “aryl” embraces aromatic radicals such as phenyl, naphthyl, tetrahydronaphthyl, indane and biphenyl. More preferred aryl is phenyl. The “aryl” group may have 1 to 3 substituents such as lower alkyl, hydroxyl, halo, haloalkyl, nitro, cyano, alkoxy, and lower alkylamino.

The term “aralkyl” embraces aryl-substituted alkyl radicals. Preferable aralkyl radicals are “lower aralkyl” radicals having aryl radicals attached to alkyl radicals having one to six carbon atoms. Even more preferred are lower aralkyl radicals phenyl attached to alkyl portions having one to three carbon atoms. Examples of such radicals include benzyl, diphenylmethyl and phenylethyl. The aryl in said aralkyl may be additionally substituted with halo, alkyl, alkoxy, haloalkyl and haloalkoxy.

The term “arylalkenyl” embraces aryl-substituted alkenyl radicals. Preferable arylalkenyl radicals are “lower arylalkenyl” radicals having aryl radicals attached to alkenyl radicals having two to six carbon atoms. Examples of such radicals include phenylethenyl. The aryl in said arylalkenyl may be additionally substituted with halo, alkyl, alkoxy, haloalkyl and haloalkoxy.

The term “N-arylaminoalkyl” denotes aminoalkyl radicals substituted with an aryl radical. More preferred arylaminoalkyl radicals are “lower N-arylaminoalkyl” radicals having alkyl radicals of one to six carbon atoms. Even more preferred are phenylaminoalkyl radicals having one to three carbon atoms. Examples of such radicals include N-phenylaminomethyl and N-phenylaminoethyl.

The term “aralkylaminoalkyl” embraces aralkyl radicals as described above, attached to an aminoalkyl radical as defined herein. More preferred are lower arylalkylaminoalkyl radicals independently having alkyl radicals of one to three carbon atoms.

The term “basic moiety” or “basic moieties” means a chemical moiety that has a measured or calculated pKₐ of from about 7 to about 13. The term also can include a chemical moiety that is protonable, to some extent, between a pH range of from about 7 to about 10. Examples of basic moieties include, but are not limited to, amino, cycloalkylamino-(C₁-C₆)alkyl, cycloalkyl(C₁-C₆)alkylamino(C₁-C₆)alkyl, heterocyclylamino(C₁-C₆)alkyl, heterocyclyl(C₁-C₆)alkylamino(C₁-C₆)alkyl, arylamino(C₁-C₆)alkyl, aryl(C₁-C₆)alkylamino-(C₁-C₆)alkyl, (C₁-C₆)alkylamino(C₁-C₆)alkoxy, (C₁-C₆)alkylamino(C₁-C₆)alkoxy(C₁-C₆)-alkoxy, amino(C₁-C₆)alkoxy, amino(C₁-C₆)alkyl, (C₁-C₆)alkylamino(C₁-C₆)alkyl, (C₁-C₄)alkylamino-(C₂-C₆)alkenyl, 4-8-membered nitrogen-containing heterocyclyl(C₂-C₆)alkenyl, 5-6 membered heterocyclyloxy, 5-6 membered nitrogen-containing heterocyclyl and 5-7 membered nitrogen-containing heterocyclylalkyl; more specifically amino, cycloalkylamino(C₁-C₆)alkyl, cycloalkyl(C₁-C₆)alkylamino-(C₁-C₆)alkyl, heterocyclylamino(C₁-C₆)alkyl, heterocyclyl(C₁-C₆)alkylamino-(C₁-C₆)alkyl, arylamino(C₁-C₆)alkyl, aryl(C₁-C₆)alkylamino(C₁-C₆)alkyl, (C₁-C₆)alkyl amino(C₁-C₆)alkoxy, (C₁-C₆)alkylamino(C₁-C₆)alkoxy(C₁-C₆)alkoxy, amino(C₁-C₆)alkoxy, amino(C₁-C₆)alkyl, (C₁-C₆)alkylamino(C₁-C₆)alkyl, (C₁-C₄)alkylamino-(C₂-C₆)alkenyl, 5-8-membered nitrogen-containing heterocyclyl(C₂-C₆)alkenyl, heterocyclyl(C₁-C₆)amino(C₂-C₆)alkyl, 5-6 membered heterocyclyloxy, 5-6 membered nitrogen-containing heterocyclyl and 5-7 membered nitrogen-containing heterocyclyl(C₁-C₆)alkyl. Each basic moiety can be substituted by 0, 1, 2 or 3 groups independently selected from halo, —NH₂, —OH, —CN, —CF₃, (C₁-C₆)alkylamino, haloalkyl, oxo, (C₁-C₆)alkoxy, (C₁-C₆)alkoxyalkyl, (C₂-C₆)alkenyl, (C₂-C₆)alkynyl, di(C₁-C₆)alkylamino, ═NCN; and (C₁-C₆)alkyl, aryl, heteroaryl, cycloalkyl and heterocyclyl, each of which is substituted by 0, 1, 2 or 3 groups independently selected from halo, —NH₂, —OH, —CN, —CF₃, (C₁-C₆)alkylamino, haloalkyl, oxo, (C₁-C₆)alkoxy, (C₁-C₆)alkoxyalkyl, (C₁-C₆)alkyl, (C₂-C₆)alkenyl, (C₂-C₆)alkynyl, or di(C₁-C₆)alkylamino. In one emodiment, the basic moiety is selected from cycloalkylamino(C₁-C₆)alkyl, cycloalkyl(C₁-C₆) alkylamino(C₁-C₆)alkyl, heterocyclylamino(C₁-C₆)alkyl, heterocyclyl(C₁-C₆)alkyl-amino(C₁-C₆)alkyl, arylamino(C₁-C₆)alkyl, aryl(C₁-C₆)alkylamino(C₁-C₆)alkyl, (C₁-C₆)alkyl amino(C₁-C₆)alkoxy, (C₁-C₆)alkylamino(C₁-C₆)alkoxy(C₁-C₆)alkoxy, amino(C₁-C₆)alkoxy, amino(C₁-C₆)alkyl, (C₁-C₆)alkylamino(C₁-C₆)alkyl, (C₁-C₄)alkylamino-(C₂-C₆)alkenyl, 4-8-membered nitrogen-containing heterocyclyl(C₂-C₆)alkenyl, heterocyclyl(C₁-C₆)amino-(C₂-C₆)alkyl, 5-6 membered heterocyclyloxy, 5-6 membered nitrogen-containing heterocyclyl and 5-7 membered nitrogen-containing heterocyclylalkyl. In another emodiment, the basic moiety is selected from cycloalkylamino(C₁-C₆)alkyl, cycloalkyl(C₁-C₆) alkylamino-(C₁-C₆)alkyl, heterocyclylamino(C₁-C₆)alkyl, heterocyclyl(C₁-C₆)alkylamino(C₁-C₆)alkyl, arylamino(C₁-C₆)alkyl, aryl(C₁-C₆)alkylamino(C₁-C₆)alkyl, (C₁-C₆)alkyl amino(C₁-C₆)alkoxy, (C₁-C₆)alkylamino(C₁-C₆)alkoxy(C₁-C₆)alkoxy, amino(C₁-C₆)alkoxy, amino(C₁-C₆)alkyl, (C₁-C₆)alkylamino(C₁-C₆)alkyl, (C₁-C₄)alkylamino-(C₂-C₆)alkenyl, 4-8-membered nitrogen-containing heterocyclyl(C₂-C₆)alkenyl, heterocyclyl(C₁-C₆)amino(C₂-C₆)alkyl, 5-6 membered heterocyclyloxy, 5-6 membered nitrogen-containing heterocyclyl and 5-7 membered nitrogen-containing heterocyclyl-alkyl any of which are substituted by halo, C₁₋₆alkyl or cycloalkyl, preferably halo, C₁₋₆alkyl or cycloalkyl. More specifically, the basic moiety is amino, aminomethyl, isopropylaminomethyl, t-butylaminomethyl, 2-t-butylaminoethyl, 2-tert-butylamino-1-methyl-ethyl, 1-tert-butylaminoethyl, 1-(tert-butylamino-methyl)-vinyl, 1-(piperidin-1-ylmethyl)-vinyl, N-isobutyl-aminomethyl, N-isobutyl-aminoethyl, (2,2-dimethyl)propylaminomethyl, N-isopropyl-N-ethylaminomethyl, N-isopropyl-N-methylaminomethyl, N-t-butyl-N-methylaminomethyl, N-iso-butyl-N-methylaminomethyl, N-t-butyl-N-ethylaminomethyl, N-isobutyl-N-methylaminomethyl, N-t-butyl-N-isopropylaminomethyl, N,N-di(isopropyl)aminomethyl, N,N-dimethylaminomethyl, N,N-diethylaminomethyl, N,N-di(t-butyl)-aminomethyl, cyclopropylaminomethyl, cyclopropylaminoethyl, cyclopropylmethylaminomethyl, cyclopropylmethylaminoethyl, cyclobutylaminomethyl, cyclobutylaminoethyl, cyclobutylmethylaminomethyl, cyclobutylmethylaminoethyl, 4,5-dihydro-imidazolyl, 1-piperidinylmethyl, 4-fluoropiperidin-1-ylmethyl, 4,4-difluoropiperidin-1-ylmethyl, 3-hydroxypiperidin-1-ylmethyl, 4-hydroxy-piperidin-1-ylmethyl, 4-(piperidin-1-yl)piperidinylmethyl, 4-(dimethylamino)piperidin-1-ylmethyl, 2,6-dimethylpiperidin-1-ylmethyl, 4-morpholinylmethyl, 1-pyrrolidinylmethyl, 2-methylpyrrolidin-1-ylmethyl, 2,5-dimethylpyrrolidin-1-ylmethyl, piperazin-1-ylmethyl, azocan-1-ylmethyl, azepan-1-ylmethyl, (7-azabicyclo[2.2.1]hept-7-yl)methyl, (1,3,3-trimethyl-6-azaicyclo[3.2.1]oct-6-yl)methyl, 2-piperidinyl or 4-methylpiperazin-1-ylmethyl.

The term “cycloalkyl” includes saturated carbocyclic groups. Preferred cycloalkyl groups include C₃-C₆ rings. More preferred compounds include cyclopentyl, cyclopropyl, and cyclohexyl.

The term “cycloalkylaminoalkyl” refers to aminoalkyl radicals where the nitrogen atom of the amino group is independently substituted with one or two cycloalkyl radicals and therefore includes “N-cycloalkylaminoalkyl” and “N,N-dicycloalkylaminoalkyl”. More preferred cycloalkylaminoalkyl radicals are “lower cycloalkylaminoalkyl” radicals having alkyl radicals with one to six carbon atoms. Even more preferred are lower cycloalkylaminoalkyl radicals having alkyl radicals with one to three carbon atoms. Examples of such lower alkylaminosulfonyl radicals include N-cyclohexylaminomethyl- and N-cyclopentylaminoethyl.

The term “cycloalkylalkylaminoalkyl” embraces cycloalkyl radicals as described above, attached to an alkylaminoalkyl radical. More preferred are lower cycloalkyl-alkylaminoalkyl radicals independently having alkyl radicals of one to three carbon atoms.

“Halo” or “halogen” means a halogen atoms selected from F, Cl, Br and I.

“C_{α-β}haloalkyl” means an alkyl group as described above, unless otherwise indicated, wherein any number—at least one—of the hydrogen atoms attached to the alkyl chain are replaced by F, Cl, Br or I.

“Heterocycle” or “heterocyclyl” means a ring comprising at least one carbon atom and at least one other atom selected from N, O and S. The term heterocycle embraces saturated, partially saturated and unsaturated heteroatom-containing ring radicals, where the heteroatoms may be selected from nitrogen, sulfur and oxygen. It does not include rings containing —O—O— or —S—S— portions. Preferably, the heterocycle ring contains 3 to 10 ring atoms. Unsaturated heteroatom-containing ring radicals as used herein means a heterocycle containing at least one aromatic ring. Unsaturated heteroatom-containing ring radicals are also referred to herein as heteroaryl. Partially saturated heteroatom-containing ring radicals as used herein means a heterocycle containing one or more double bonds provided that it is not aromatic.

Examples of heterocycles that may be found in the claims include, but are not limited to, the following:

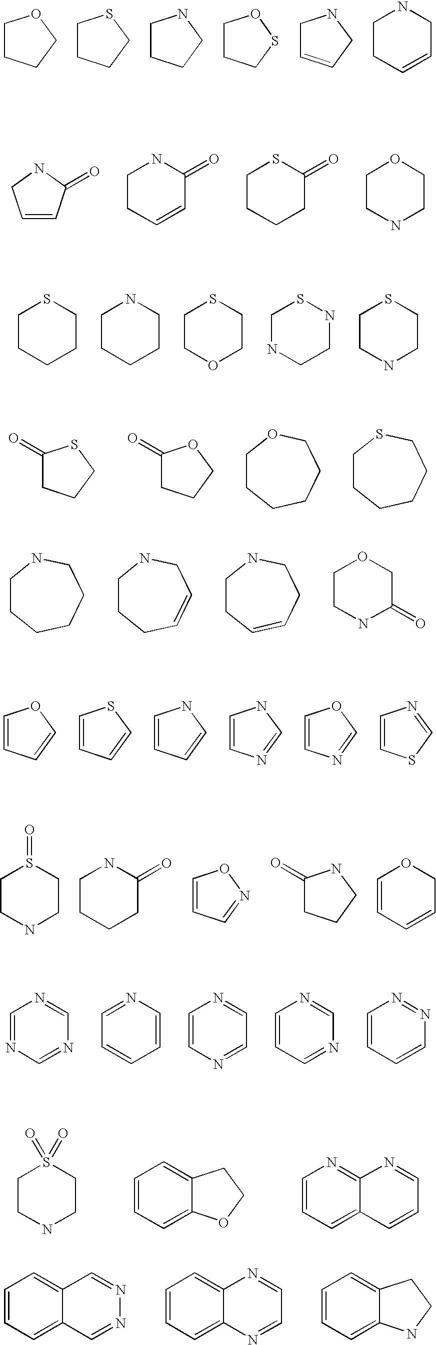

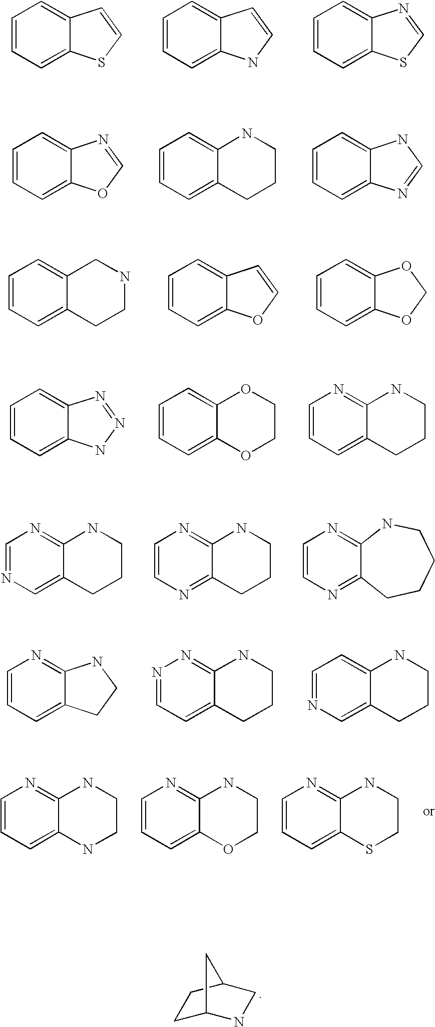

The term “heterocyclylaminoalkyl” embraces heterocyclyl radicals as described above, attached to an aminoalkyl radical as defined herein.

The term “heterocyclylalkylaminoalkyl” embraces heterocyclylalkyl radicals as described below, attached to an aminoalkyl radical. More preferred are lower heterocyclylalkylaminoalkyl radicals having, independently, alkyl radicals of one to three carbon atoms.

The term “heterocyclylalkyl” embraces heterocycle-substituted alkyl radicals. More preferred heterocyclylalkyl radicals are “5- or 6-membered heteroarylalkyl” radicals having alkyl portions of one to six carbon atoms and a 5- or 6-membered heteroaryl radical. Even more preferred are lower heteroarylalkyl radicals having alkyl portions of one to three carbon atoms. Examples include such radicals as pyridinylmethyl and thienylmethyl.

The term “heterocyclylalkenyl” embraces heterocyclyl-substituted alkenyl radicals. Preferable heterocyclylalkenyl radicals are “lower heterocyclylalkenyl” radicals having heterocyclyl radicals attached to alkenyl radicals having two to six carbon atoms. Examples of such radicals include pyridinylethenyl. When the heterocyclyl ring contains 4 to 8 ring atoms having at least a nitrogen ring atom it is referred to herein as 4-8 membered nitrogen containing heterocyclylalkenyl.

The term “heterocyclyloxy” embraces optionally substituted heterocyclyl radicals, as defined above, attached to an oxygen atom. Examples of such radicals include piperidyloxy.

The term “H” denotes a single hydrogen atom. This radical may be attached, for example, to an oxygen atom to form a hydroxyl radical.

The terms “oxo” represent the group ═O (as in carbonyl).

The term “hydroxyalkyl” embraces linear or branched alkyl radicals having one to about ten carbon atoms any one of which may be substituted with one or more hydroxyl radicals.

The terms “carboxy” or “carboxyl” denotes —CO₂H.

The term “carbonyl” denotes —(C═O)—.

The term “comprising” is meant to be open ended, including the indicated component but not excluding other elements.

The specification and claims contain listing of species using the language “selected from . . . and . . . ” and “is . . . or . . . ” (sometimes referred to as Markush groups). When this language is used in this application, unless otherwise stated it is meant to include the group as a whole, or any single members thereof, or any subgroups thereof. The use of this language is merely for shorthand purposes and is not meant in any way to limit the removal of individual elements or subgroups from the genus.

The term “ . . . a saturated 5-, 6- or 7-membered monocyclic or 6-, 7-, 8-, 9-, 10- or 11-membered bicyclic hydrocarbon ring . . . ” means a hydrocarbon ring that do not contain a double bond.

The term “ . . . a partially saturated 5-, 6- or 7-membered monocyclic or 6-, 7-, 8-, 9-, 10- or 11-membered bicyclic hydrocarbon ring . . . ” means a hydrocarbon ring that contain one or more double bonds provided that they are not aromatic.

The term “ . . . a unsaturated 5-, 6- or 7-membered monocyclic or 6-, 7-, 8-, 9-, 10- or 11-membered bicyclic hydrocarbon ring . . . ” means a hydrocarbon ring where at least one of the rings is aromatic.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

The phrase “therapeutically-effective” is intended to qualify the amount of each agent, which will achieve the goal of improvement in disorder severity and the frequency of incidence over treatment of each agent by itself, while avoiding adverse side effects typically associated with alternative therapies. For example, effective pain therapeutic agents relieve the pain sensation of the patient. Alternatively, effective therapeutic agents for the treatment of inflammation minimize the damage from the inflammation, and the like.

The term “treatment” includes therapeutic treatment as well as prophylactic treatment (either preventing the onset of disorders altogether or delaying the onset of a pre-clinically evident stage of disorders in individuals).

Compounds of the present invention can possess, in general, tautomeric forms, including any enolate anions such as cyclic and acyclic amidine and guanidine groups, heteroatom substituted heterocyclyl groups (Y′=O, S, NR), and the like, which are illustrated in the following examples. All such forms are within the scope of this invention.

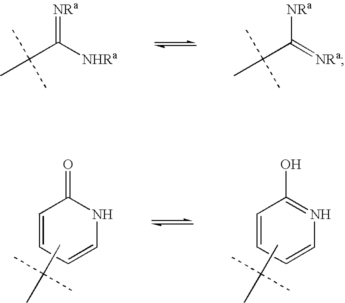

The compounds may also occur in cis- or trans- or E- or Z-double bond isomeric forms. All such isomeric forms of such compounds are included in the present invention. All crystal forms of the compounds described herein are expressly included in the present invention. Additionally, all crystal forms of the compounds described herein are expressly included in the present invention.

Substituents on ring moieties (e.g., phenyl, thienyl, etc.) may be attached to specific atoms, whereby they are intended to be fixed to that atom, or they may be drawn unattached to a specific atom, whereby they are intended to be attached at any available atom that is not already substituted by an atom other than H (hydrogen).

The compounds of this invention may contain heterocyclic ring systems attached to another ring system. Such heterocyclic ring systems may be attached through a carbon atom or a heteroatom in the ring system.

Compounds of the present invention can possess, in general, one or more asymmetric carbon atoms and are thus capable of existing in the form of optical isomers as well as in the form of racemic or non-racemic mixtures thereof. Unless otherwise indicated, the compounds of the present invention, as depicted or named, may exist as the racemate, a single enantiomer, or any uneven (i.e. non 50/50) mixture of enantiomers. All such isomeric forms are within the scope of the invention. The optical isomers can be obtained by resolution of the racemic mixtures according to conventional processes, e.g., by formation of diastereoisomeric salts, by treatment with an optically active acid or base. Examples of appropriate acids are tartaric, diacetyltartaric, dibenzoyltartaric, ditoluoyltartaric, and camphorsulfonic acid and then separation of the mixture of diastereoisomers by crystallization followed by liberation of the optically active bases from these salts. A different process for separation of optical isomers involves the use of a chiral chromatography column, such as, for example, a CHIRAL-AGP column, optimally chosen to maximize the separation of the enantiomers. Still another available method involves synthesis of covalent diastereoisomeric molecules by reacting compounds of the invention with an optically pure acid in an activated form or an optically pure isocyanate. The synthesized diastereoisomers can be separated by conventional means such as chromatography, distillation, crystallization or sublimation, and then hydrolyzed to deliver the enantiomerically pure compound. The optically active compounds of the invention can likewise be obtained by using optically active starting materials. These isomers may be in the form of a free acid, a free base, an ester or a salt. Preferred compounds of the invention have an R configuration at the amide bond as shown below

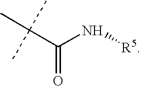

Also included in the family of compounds of Formula (I) are the pharmaceutically-acceptable salts thereof. The term “pharmaceutically-acceptable salts” embraces salts commonly used to form alkali metal salts and to form addition salts of free acids or free bases. The nature of the salt is not critical, provided that it is pharmaceutically-acceptable. Suitable pharmaceutically-acceptable acid addition salts of compounds of Formula I may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, arylaliphatic, heterocyclic carboxylic and sulfonic classes of organic acids, example of which are formic, acetic, adipic, butyric, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, camphoric, camphorsulfonic, digluconic, cyclopentanepropionic, dodecylsulfonic, glucoheptanoic, glycerophosphonic, heptanoic, hexanoic, 2-hydroxy-ethanesulfonic, nicotinic, 2-naphthalenesulfonic, oxalic, palmoic, pectinic, persulfuric, 2-phenylpropionic, picric, pivalic propionic, succinic, tartaric, thiocyanic, mesylic, undecanoic, stearic, algenic, β-hydroxybutyric, salicylic, galactaric and galacturonic acid. Suitable pharmaceutically-acceptable base addition salts of compounds of Formula I include metallic salts, such as salts made from aluminum, calcium, lithium, magnesium, potassium, sodium and zinc, or salts made from organic bases including primary, secondary and tertiary amines, substituted amines including cyclic amines, such as caffeine, arginine, diethylamine, N-ethyl piperidine, histidine, glucamine, isopropylamine, lysine, morpholine, N-ethylmorpholine, piperazine, piperidine, triethylamine, trimethylamine. All of these salts may be prepared by conventional means from the corresponding compound of the invention by reacting, for example, the appropriate acid or base with the compound of Formula I

Also, the basic nitrogen-containing groups can be quaternized with such agents as lower alkyl halides, such as methyl, ethyl, propyl, and butyl chloride, bromides and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others. Water or oil-soluble or dispersible products are thereby obtained.

Examples of acids that may be employed to from pharmaceutically acceptable acid addition salts include such inorganic acids as HCl, H₂SO₄ and H₃PO₄ and such organic acids as oxalic acid, maleic acid, succinic acid and citric acid. Other examples include salts with alkali metals or alkaline earth metals, such as sodium, potassium, calcium or magnesium or with organic bases.

### General Synthetic Procedures

Compounds of this invention can be made by the methods depicted in the reaction schemes shown below.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1994); Rodd's Chemistry of Carbon Compounds, Volumes 1-5 and Supplementals (Elsevier Science Publishers); Organic Reactions, Volumes 1-46 (John Wiley and Sons, 2003), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure.

The starting materials and the intermediates of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., more preferably from about 0° C. to about 125° C. and most preferably at about room (or ambient) temperature, e.g., about 20° C.

In the reactions described hereinafter it may be necessary to protect reactive functional groups, for example hydroxy, amino, imino, thio or carboxy groups, where these are desired in the final product, to avoid their unwanted participation in the reactions. Conventional protecting groups may be used in accordance with standard practice, for examples see T. W. Greene and P. G. M. Wuts in “Protective Groups in Organic Chemistry” John Wiley and Sons, 1999. Compounds of this invention can be made by the methods depicted in the reaction schemes shown below.

Compounds of Formula (I) where R¹, R², R^{1a}, R^{1b}, R^{1c}, R^{3a}, R^{3b}, R⁴ and R⁵ are as defined in the Summary of the Invention can be prepared as shown in Scheme A below.

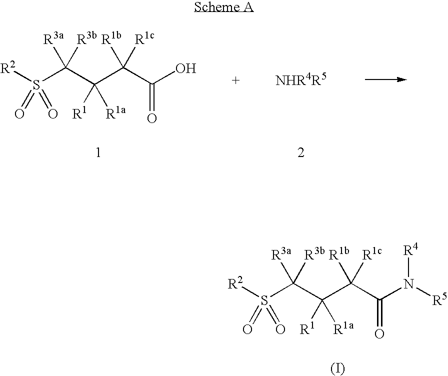

Reaction of a sulfonyl compound of formula I where R², R^{1a}, R^{1b}, R^{1c}, R^{3a}, R^{3b} are as defined in the Summary of the Invention with an amine of formula 2 where R⁴ and R⁵ are as defined in the Summary of the Invention provides a compound of Formula (I). The reaction is carried out in the presence of a coupling agent such as are coupled with the substituted amine 2 using standard peptide coupling conditions coupling agent (e.g., benzotriazol-1-yloxy-trispyrrolidinophosphonium hexafluorophosphate (PyBOP®.), 1-(3-dimethylamino-propyl)-3-ethylcarbodiimide hydrochloride (EDCI), O-(7-azabenzotrizol-1-yl)-1,1,3,3, tetramethyluronium-hexafluoro-phosphate (HATU), O-benzotriazol-1-yl-N,N,N′,N′-tetramethyl-methylmorpholine, and the like, or any suitable combination thereof) at ambient temperature. Suitable reaction solvents include, but are not limited to, dimethylformamide, methylene chloride, and the like.

Compounds of formula I can be prepared by methods well known in the art. Some such methods are described below.

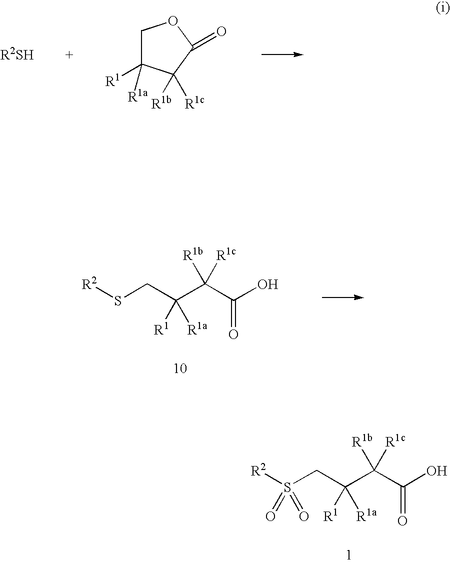

Reaction of a dihydrofuran-2(3H)-one with a thiol compound of formula R²SH where R² is as defined in the Summary of the Invention provides a compound of formula 10. The reaction is carried out in the presence of sodium hydride in a suitable organic solvent such as dimethylformamide, and the like. Dihydrofuran-2(3H)-one such as (3S,4S)-3,4-dihydroxy-dihydrofuran-2(3H)-one, (3aR,6aR)-2,2-dimethyl-dihydrofuro[3,4-d][1,3]dioxol-4(3aH)-one, (3R,4S)-3,4-dihydroxy-dihydrofuran-2(3H)-one, (3S,4R)-3,4-dihydroxy-dihydrofuran-2(3H)-one, 3,3-dimethyl-dihydrofuran-2(3H)-one, and 4-phenyldihydrofuran-2(3H)-one are commercially available. Others can be prepared by known literature methods, e.g, the method of Sengupta and Mehta, Tetrahedron Letters, 1996, 37, 8625. Oxidation of the sulfur atoms with a suitable oxidizing agent such as Oxone, and the like provides a compound of formula 1.

Compounds of formula R²SH are also commercially available.

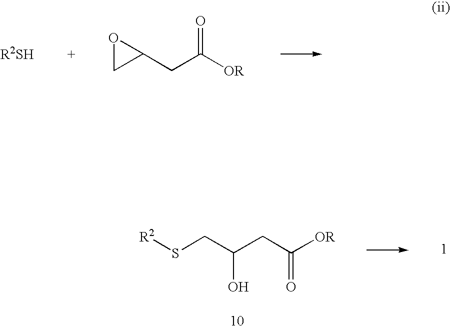

Compounds of formula I where R¹ is hydroxyl or derivatives thereof and R^{1a}, R^{1b}, R^{1c}, R^{3a}, and R^{3b} are hydrogen can be prepared by reacting oxiran-2-ylacetate with a thiol compound of formula R²SH in the presence of a base such as sodium carbonate, cesium carbonate, and the like in an alcoholic solvent such as methanol, ethanol, and the like to provide a compound of formula 10 where R^{1a}, R^{1b}, R^{1c}, R^{3a}, and R^{3b} are hydrogen. Compound 10 is then converted to a compound of formula 1 by oxidation of the sulfur atom as described above, followed by hydrolysis of the ester group to the acid. Ethyl oxiran-2-ylacetate is commercially available.

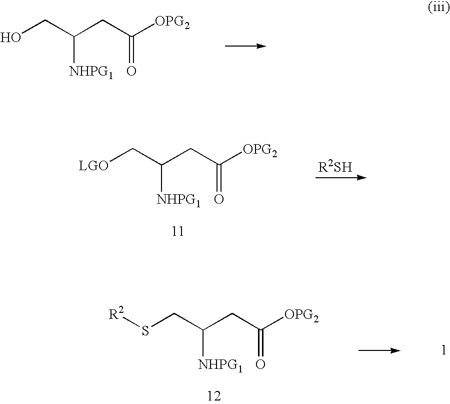

Compounds of formula I where R¹ is amino or derivatives thereof and R^{1a}, R^{1b}, R^{1c}, R^{3a}, and R^{3b} are hydrogen can be prepared by reacting 3-amino-4-hydroxybutonate where PG₁ and PG₂ are amino and hydroxyl protecting groups respectively. The 4-hydroxy group in 3-amino-4-hydroxybutanoate is first converted to a suitable leaving group such as mesylate, tosylate and the like under conditions well known in the art to give a compound of formula 11. Treatment of 11 with a thiol compound of formula R²SH under the reaction conditions described in method (i) above provides a compound of formula 12 which upon oxidation of the sulfur atom and removal of the carboxy provides a compound of formula 1. The amino protecting group can be optionally removed and modified if desired prior to proceeding further.

Detailed descriptions of syntheses of compounds of formula 1 via the above procedures and additional procedures for the synthesis of compounds of formula 1 are provided in working examples below.

Amines of formula 2 are commercially available or may be prepared by methods well known in the art. Detailed descriptions of syntheses of amines are provided in working examples below.

Alternatively, the compounds of Formula (I) where R¹, R², R^{1a}, R^{1b}, R^{1c}, R^{3a}, R^{3b}, R⁴ and R⁵ are as defined in the Summary of the Invention can be prepared as shown in Scheme B below.

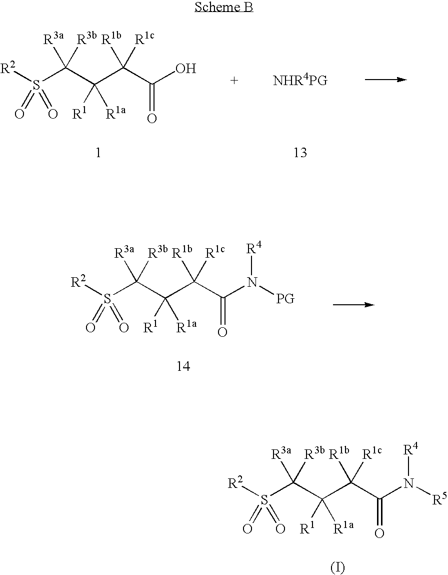

Compounds of Formula (I) can also be prepared by reacting compound 1 with an amine of formula 13 where PG is a precursor group to R⁵ group. Conversion of the PG group in compound 14 to an R⁵ group then provides a compound of Formula (I).

For example, a compound of Formula (I) where R⁵ is a saturated, partially saturated or unsaturated 8-, 9-, 10- or 11-membered bicyclic or 12-, 13-, 14- or 15-membered tricyclic ring substituted with cycloalkylamino-(C₁-C₆)alkyl, cycloalkyl(C₁-C₆)alkylamino(C₁-C₆)alkyl, heterocyclylamino(C₁-C₆)alkyl, heterocyclyl(C₁-C₆)alkylamino(C₁-C₆)alkyl, arylamino(C₁-C₆)alkyl, aryl(C₁-C₆)alkylamino-(C₁-C₆)alkyl, (C₁-C₆)alkylamino(C₁-C₆)alkoxy, (C₁-C₆)alkylamino(C₁-C₆)alkoxy(C₁-C₆)-alkoxy, amino(C₁-C₆)alkoxy, amino(C₁-C₆)alkyl, (C₁-C₆)alkylamino(C₁-C₆)alkyl, and 5-7 membered nitrogen-containing heterocyclylalkyl can be prepared from a corresponding compound of formula 14 where PG is a saturated, partially saturated or unsaturated 8-, 9-, 10- or 11-membered bicyclic or 12-, 13-, 14- or 15-membered tricyclic ring substituted with a hydroxyalkyl group by treating the hydroxyalkyl compound with an oxidizing agent such as manganese oxide and treating the resulting aldehyde compound with an amine optionally substituted with cycloalkyl, cycloalkylalkyl, heterocyclyl, alkyl, aryl, aralkyl, or nitrogen containing heterocyclyl group under reductive amination reaction conditions to provide a compound of Formula (I) with the above listed substitutents.

Alternatively, the aldehyde group can first treated with an unsubstituted amine and then the amine can be substituted under standard alkylation reaction conditions or treated with an aldehyde under reductive amination reaction conditions. The above compounds can also be prepared from a corresponding compound of formula 14 where PG is a saturated, partially saturated or unsaturated 8-, 9-, 10- or 11-membered bicyclic or 12-, 13-, 14- or 15-membered tricyclic ring substituted with an alkene group by first converting the alkyne group to an aldehyde under ozonolysis reaction conditions and then proceeding as described above.

Compounds of Formula (I) where R⁵ is a saturated, partially saturated or unsaturated 8-, 9-, 10- or 11-membered bicyclic or 12-, 13-, 14- or 15-membered tricyclic ring substituted with 5-6 membered heterocyclyloxy can be prepared from a corresponding compound of formula 14 where PG is a saturated, partially saturated or unsaturated 8-, 9-, 10- or 11-membered bicyclic or 12-, 13-, 14- or 15-membered tricyclic ring substituted with a hydroxyl group by reacting it with heterocyclyl halide under alkylating reaction conditions.

Alternatively, the compounds of Formula (I) where R^{3a}, R^{3b}, R^{1a}, and R^{1b} are hydrogen, R¹ and R^{1c} are hydroxyl or hydroxy derivatives listed in the Summary of the Invention and R², R⁴ and R⁵ are as defined in the Summary of the Invention can be prepared as shown in Scheme C below.

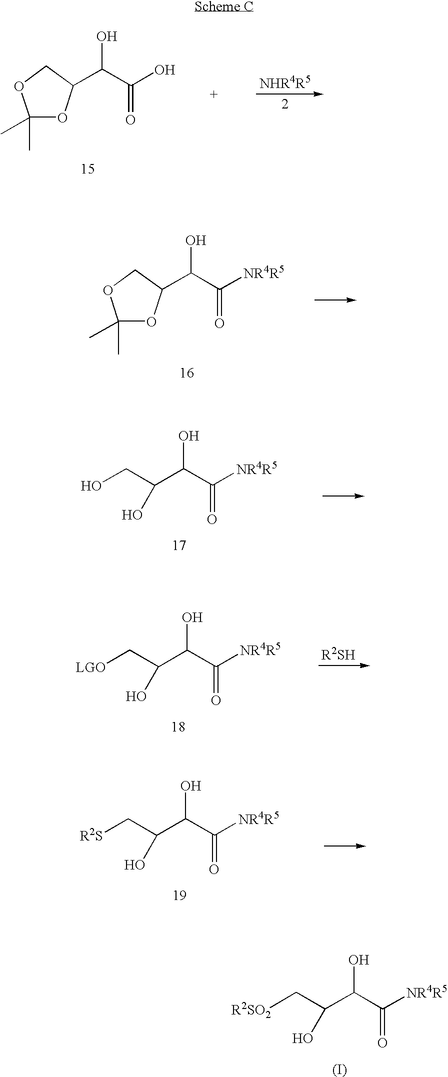

Compounds of Formula (I) where R^{3a}, R^{3b}, R^{1a}, and R^{1b} are hydrogen, R¹ and R^{1c} are hydroxyl or hydroxy derivatives listed in the Summary of the Invention and R², R⁴ and R⁵ are as defined in the Summary of the Invention can be prepared from commercially available protected carbohydrates 15 as shown above. Treatment of compound 15 with an amine of formula 2 provides a compound of formula 16 which upon treatment with an acid such as hydrochloric acid and the like provides a trihydroxy compound of formula 17. Treatment of 17 with tosyl chloride, mesyl chloride, and the like provides a compound of formula 18 where LG is a leaving group. The reaction is carried out in the presence of a base such as triethylamine, pyridine, and the like and in a suitable organic solvent such as methylene chloride, tetrahydrofuran, and the like.

Treatment of compound 18 with a thiol compound of formula R²SH where R² is as defined in the Summary of the Invention provides compound 19 which upon treatment with an oxidizing agent such as Oxone, m-chloroperbenzoic acid, and the like provides a compound of Formula (I) where R¹ and R^{1c} are hydroxyl. Compounds of Formula (I) can be converted to compound of Formula (I) where R¹ and R^{1c} are hydroxyl derivatives listed in the Summary of the Invention by methods well known in the art.

It will be well recognized by a person skilled in the art that if one or more functional groups, for example carboxy, hydroxy, amino, or mercapto, need to be protected during the synthesis of a compound of Formula (I) because they should not take part in the reaction. Such groups should be suitably protected prior to or during the synthesis procedure. A person skilled in the art would be able to easily establish, which protecting groups are suitable with the reactions mentioned above. The protection of such functional groups by protecting groups and their removal reactions are described for example in standard reference works, such as J. F. W. McOmie, “Protective Groups in Organic Chemistry”, Plenum Press, London and New York 1973, in T. W. Greene, “Protective Groups in Organic Synthesis”, Wiley, New York 1991, in “The Peptides”; Volume 3 (editors: E. Gross and J. Meienhofer), Academic Press, London and New York 1981, in “Methoden der organischen Chemie” (Methods of organic chemistry), Houben Weyl, 4th edition, Volume 15/1, Georg Thieme Verlag, Stuttgart 1974, in H.-D. Jakubke and H. Jescheit, “Aminosäuren, Peptide, Proteine” (Amino acids, peptides, proteins), Verlag Chemie, Weinheim, Deerfield Beach, and Basel 1982, and in Jochen Lehmann, “Chemie der Kohlenhydrate: Monosaccharide und Derivate” (Chemistry of carbohydrates: monosaccharides and derivatives), Georg Thieme Verlag, Stuttgart 1974.

Salts of a compound of Formula (I) with a salt-forming group may be prepared in a manner known per se. Acid addition salts of compounds of Formula (I) may thus be obtained by treatment with an acid or with a suitable anion exchange reagent. A salt with two acid molecules (for example a dihalogenide of a compound of Formula (I)) may also be converted into a salt with one acid molecule per compound (for example a monohalogenide); this may be done by heating to a melt, or for example by heating as a solid under a high vacuum at elevated temperature, for example from 130-170° C., one molecule of the acid being expelled per molecule of a compound of Formula (I).

Salts can usually be converted to free compounds, e.g. by treating with suitable basic agents, for example with alkali metal carbonates, alkali metal hydrogen carbonates, or alkali metal hydroxides, typically potassium carbonate or sodium hydroxide.

The compounds of Formula (I), including their salts, are also obtainable in the form of hydrates, or their crystals can include for example the solvent used for crystallization (present as solvates). All such forms are within the scope of this invention.

As can be appreciated by the skilled artisan, the above synthetic schemes are not intended to comprise a comprehensive list of all means by which the compounds described and claimed in this application may be synthesized. Further methods will be evident to those of ordinary skill in the art. Additionally, the various synthetic steps described above may be performed in an alternate sequence or order to give the desired compounds.

### Utility

The compound of Formula (I) are B1 receptor antagonists and hence are useful in the treatment of a disorder such as acute pain, dental pain, back pain, lower back pain, pain from trauma, surgical pain, pain resulting from amputation or abscess, causalgia, fibromyalgia, demyelinating diseases, trigeminal neuralgia, cancer, chronic alcoholism, stroke, thalamic pain syndrome, diabetes, acquired immune deficiency syndrome (“AIDS”), toxins and chemotherapy, general headache, migraine, cluster headache, mixed-vascular and non-vascular syndromes, tension headache, general inflammation, arthritis, rheumatic diseases, lupus, osteoarthritis, inflammatory bowel disorders, inflammatory eye disorders, inflammatory or unstable bladder disorders, psoriasis, skin complaints with inflammatory components, sunburn, carditis, dermatitis, myositis, neuritis, collagen vascular diseases, chronic inflammatory conditions, inflammatory pain and associated hyperalgesia and allodynia, neuropathic pain and associated hyperalgesia and allodynia, diabetic neuropathy pain, sympathetically maintained pain, deafferentation syndromes, asthma, vasomotor or allergic rhinitis, epithelial tissue damage or dysfunction, herpes simplex, post-herpetic neuralgia, disturbances of visceral motility at respiratory, genitourinary, gastrointestinal or vascular regions, wounds, burns, allergic skin reactions, pruritis, vitiligo, general gastrointestinal disorders, colitis, inflammatory bowel disease, gastric ulceration, duodenal ulcers, thalamic pain syndrome, diabetes, toxins and chemotherapy, septic shock, and bronchial disorders.

### Biological Testing

The in vitro binding affinity of the compounds of the invention to the human B1 and B2 bradykinin receptors can be tested using the radioligand binding assay described in Biological Example 1 below. The antagonistic activity of the compounds of the invention for the human B1 and B2 bradykinin receptors can be tested using the calcium flux assay, Rabbit endothelial cell B1-specific PGI₂ secretion Assay, and umbilical vein Assay described in Biological Examples 2 and 3 below. The antinociceptive activity of the compounds of the invention was determined using the rat and monkey pain models described in Example 4 below. The antiinflammatory activity of the compounds of the invention was determined using the Green Monkey LPS inflammation model described in Example 5 below.

### Pharmaceutical Compositions and Administration

The present invention also embraces pharmaceutical compositions comprising the active compounds of Formula (I) in association with one or more non-toxic, pharmaceutically-acceptable carriers and/or diluents and/or adjuvants (collectively referred to herein as “carrier” materials) and, if desired, other active ingredients. The active compounds of the present invention may be administered by any suitable route, preferably in the form of a pharmaceutical composition adapted to such a route, and in a dose effective for the treatment intended. The compounds and compositions of the present invention may, for example, be administered orally, mucosally, topically, rectally, pulmonarily such as by inhalation spray, or parentally including intravascularly, intravenously, intraperitoneally, subcutaneously, intramuscularly intrasternally and infusion techniques, in dosage unit formulations containing conventional pharmaceutically acceptable carriers, adjuvants, and vehicles.

The pharmaceutically active compounds of this invention can be processed in accordance with conventional methods of pharmacy to produce medicinal agents for administration to patients, including humans and other mammals.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are tablets or capsules. For example, these may contain an amount of active ingredient from about 1 to 2000 mg, preferably from about 1 to 500 mg or 5 to 1000 mg. A suitable daily dose for a human or other mammal may vary widely depending on the condition of the patient and other factors, but, once again, can be determined using routine methods.

The amount of compounds which are administered and the dosage regimen for treating a disease condition with the compounds and/or compositions of this invention depends on a variety of factors, including the age, weight, sex and medical condition of the subject, the type of disease, the severity of the disease, the route and frequency of administration, and the particular compound employed. Thus, the dosage regimen may vary widely, but can be determined routinely using standard methods. A daily dose of about 0.01 to 500 mg/kg, preferably between about 0.1 and about 50 mg/kg, and more preferably about 0.1 and about 20 mg/kg body weight may be appropriate. The daily dose can be administered in one to four doses per day.

For therapeutic purposes, the active compounds of this invention are ordinarily combined with one or more adjuvants appropriate to the indicated route of administration. If administered, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose.

In the case of psoriasis and other skin conditions, it may be preferable to apply a topical preparation of compounds of this invention to the affected area two to four times a day.

Formulations suitable for topical administration include liquid or semi-liquid preparations suitable for penetration through the skin (e.g., liniments, lotions, ointments, creams, or pastes) and drops suitable for administration to the eye, ear, or nose. A suitable topical dose of active ingredient of a compound of the invention is 0.1 mg to 150 mg administered one to four, preferably one or two times daily. For topical administration, the active ingredient may comprise from 0.001% to 10% w/w, e.g., from 1% to 2% by weight of the formulation, although it may comprise as much as 10% w/w, but preferably not more than 5% w/w, and more preferably from 0.1% to 1% of the formulation.

When formulated in an ointment, the active ingredients may be employed with either paraffinic or a water-miscible ointment base. Alternatively, the active ingredients may be formulated in a cream with an oil-in-water cream base. If desired, the aqueous phase of the cream base may include, for example at least 30% w/w of a polyhydric alcohol such as propylene glycol, butane-1,3-diol, mannitol, sorbitol, glycerol, polyethylene glycol and mixtures thereof. The topical formulation may desirably include a compound which enhances absorption or penetration of the active ingredient through the skin or other affected areas. Examples of such dermal penetration enhancers include DMSO and related analogs.

The compounds of this invention can also be administered by a transdermal device. Preferably transdermal administration will be accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent may also function as the membrane.

The oily phase of the emulsions of this invention may be constituted from known ingredients in a known manner. While the phase may comprise merely an emulsifier, it may comprise a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. Emulsifiers and emulsion stabilizers suitable for use in the formulation of the present invention include Tween 60, Span 80, cetostearyl alcohol, myristyl alcohol, glyceryl monostearate, sodium lauryl sulfate, glyceryl distearate alone or with a wax, or other materials well known in the art.

The choice of suitable oils or fats for the formulation is based on achieving the desired cosmetic properties, since the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters may be used. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredients are dissolved or suspended in suitable carrier, especially an aqueous solvent for the active ingredients. The active ingredients are preferably present in such formulations in a concentration of 0.5 to 20%, advantageously 0.5 to 10% and particularly about 1.5% w/w.

Formulations for parenteral administration may be in the form of aqueous or non-aqueous isotonic sterile injection solutions or suspensions. These solutions and suspensions may be prepared from sterile powders or granules using one or more of the carriers or diluents mentioned for use in the formulations for oral administration or by using other suitable dispersing or wetting agents and suspending agents. The compounds may be dissolved in water, polyethylene glycol, propylene glycol, ethanol, corn oil, cottonseed oil, peanut oil, sesame oil, benzyl alcohol, sodium chloride, tragacanth gum, and/or various buffers. Other adjuvants and modes of administration are well and widely known in the pharmaceutical art. The active ingredient may also be administered by injection as a composition with suitable carriers including saline, dextrose, or water, or with cyclodextrin (ie. Captisol), cosolvent solubilization (ie. propylene glycol) or micellar solubilization (i.e., Tween 80).

The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed, including synthetic mono- or diglycerides. In addition, fatty acids such as oleic acid find use in the preparation of injectables.

For pulmonary administration, the pharmaceutical composition may be administered in the form of an aerosol or with an inhaler including dry powder aerosol.

Suppositories for rectal administration of the drug can be prepared by mixing the drug with a suitable non-irritating excipient such as cocoa butter and polyethylene glycols that are solid at ordinary temperatures but liquid at the rectal temperature and will therefore melt in the rectum and release the drug.

The pharmaceutical compositions may be subjected to conventional pharmaceutical operations such as sterilization and/or may contain conventional adjuvants, such as preservatives, stabilizers, wetting agents, emulsifiers, buffers etc. Tablets and pills can additionally be prepared with enteric coatings. Such compositions may also comprise adjuvants, such as wetting, sweetening, flavoring, and perfuming agents.

While the compounds of the invention can be administered as the sole active pharmaceutical agent, they can also be used in combination with one or more compounds of the invention or other agents. When administered as a combination, the therapeutic agents can be formulated as separate compositions that are administered at the same time or sequentially at different times, or the therapeutic agents can be given as a single composition.

The phrase “co-therapy” (or “combination-therapy”), in defining use of a compound of the present invention and another pharmaceutical agent, is intended to embrace administration of each agent in a sequential manner in a regimen that will provide beneficial effects of the drug combination, and is intended as well to embrace co-administration of these agents in a substantially simultaneous manner, such as in a single capsule having a fixed ratio of these active agents or in multiple, separate capsules for each agent.

The present compounds may also be used in combination therapies with opioids and other anti-pain analgesics, including narcotic analgesics, Mu receptor antagonists, Kappa receptor antagonists, non-narcotic (i.e. non-addictive) analgesics, monoamine uptake inhibitors, adenosine regulating agents, cannabinoid derivatives, Substance P antagonists, neurokinin-1 receptor antagonists, COX-2 inhibitors such as celecoxib, rofecoxib, valdecoxib, parecoxib, and darecoxib, NSAID's, and sodium channel blockers, among others. More preferred would be combinations with compounds selected from morphine, meperidine, codeine, pentazocine, buprenorphine, butorphanol, dezocine, meptazinol, hydrocodone, oxycodone, methadone, tetrahydrocannibinol, pregabalin, Tramadol [(+) enantiomer], DuP 747, Dynorphine A, Enadoline, RP-60180, HN-11608, E-2078, ICI-204448, acetominophen (paracetamol), propoxyphene, nalbuphine, E-4018, filenadol, mirtentanil, amitriptyline, DuP631, Tramadol [(−) enantiomer], GP-531, acadesine, AKI-1, AKI-2, GP-1683, GP-3269, 4030W92, tramadol racemate, Dynorphine A, E-2078, AXC3742, SNX-111, ADL2-1294, ICI-204448, CT-3, CP-99,994, and CP-99,994.

Alternatively, the present compounds may also be used in co-therapies with other treatments for inflammation, e.g. steroids, NSAIDs, iNOS inhibitors, p38 inhibitors, TNF inhibitors, 5-lipoxygenase inhibitors, LTB₄ receptor antagonists and LTA₄ hydrolase inhibitors.

### EXAMPLES

In order that the invention described herein may be more readily understood, the following examples are set forth. These detailed descriptions fall within the scope, and serve to exemplify, the above-described General Synthetic Procedures which form part of the invention. These detailed descriptions are presented for illustrative purposes only and are not intended as a restriction on the scope of the invention. Unless otherwise noted, all materials were obtained from commercial suppliers and used without further purification. All parts are by weight unless otherwise indicated. All compounds showed NMR spectra consistent with their assigned structures. Melting points were determined on a Buchi apparatus and are uncorrected. Mass spectral data was determined by electrospray ionization technique. All examples were purified to >95% purity as determined by high-performance liquid chromatography. Unless otherwise stated, reactions were run at RT.
AcOH, HOAc—acetic acid
AIBN—2,2′-azobisisobutyronitrile
BH₃ SMe₂—borane-methyl sulfide complex
BH₃—borane
Br₂—bromine
CBS—Corey-Bakshi-Shibata Catalyst
CCl₄—carbon tetrachloride
CH₂Cl₂—dichloromethane; DCM
CH₃CN—acetonitrile
CHCl₃—chloroform
DBU—1,8-diazabicyclo[5.4.0]undec-7-ene
DCE—1,2-dichloroethane
DMAP—4-(dimethylamino)pyridine
DMF—dimethylformamide
DMSO—dimethyl sulfoxide (also known as methyl sulfoxide)
DPPA—diphenylphosphoryl azide
EDC, EDCI—(3-dimethylamino-propyl)-ethyl carbodiimide-HCl salt
Et₂O—diethyl ether
EtOAc—ethyl acetate
EtOH—ethanol
g—gram
h—hour
H₂—hydrogen
H₂O—water
H₂SO₄—sulfuric acid
H₃PO₄—phosphoric acid
HATU—O-(7-Azabenzotriazol-1-yl)-N,N,N′,N′-tetramethyluronium hexafluorophosphate
HCl—hydrochloric acid
HCO₂H—formic acid
HOAt—1-hydroxy-7-azabenzotriazole
HOBt—1-hydroxybenzotriazole
Ip₂NEt, DIEA—diisopropylethylamine
IPA—isopropanol
iPrOH—isopropanol
ISCO—ISCO liquid chromatography system
K₂CO₃—potassium carbonate
KCN—potassium cyanide
KOH—potassium hydroxide
LAH—lithium aluminum hydride
LDA—lithium diisopropylamide
LiOH—lithium hydroxide
Me₂NH—dimethylamine
MeOH—methanol
MgSO₄—magnesium sulfate
min—minutes
mL—milliliter
N₂—nitrogen
NaBH(OAc)₃—sodium triacetoxyborohydride
NaBH₄—sodium borohydride
NaHCO₃—sodium bicarbonate
NaN₃—sodium azide
NaOAc—sodium acetate
NaOH—sodium hydroxide
NBS—N-bromosuccinimide
NH₃—ammonia
NH₄Cl—ammonium chloride
NH₄OH—ammonium hydroxide
NMM—N-methylmorpholine
NMP—1-methyl-2-pyrrolidone
Pd(OH)₂—palladium hydroxide
Pd/C—palladium on carbon
PPh₃—triphenylphosphine
(PPh₃)₂NiBr₂—bis(triphenylphosphine)nickel(II) bromide
RT—room temperature
SiO₂—silica
SOCl₂—thionyl chloride
TEA, Et₃N—triethylamine
TFA—trifluoroacetic acid
THF—tetrahydrofuran
TsCl—p-tosyl chloride
TsOH—p-toluene sulfonic acid

### Reference 1

### Synthesis of (5(R)-amino-5,6,7,8-tetrahydronaphthalen-2-yl)methanol

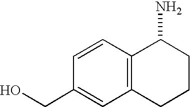

### Step A: Synthesis of 5(S)-hydroxy-5,6,7,8-tetrahydro-naphthalene-2-carboxylic acid methyl ester

To an oven-dried 2 L round-bottomed flask equipped with an argon inlet/outlet and magnetic stirring was added (R)-2-methyl-CBS-oxazaborolidine (7.4 mL of a 1M soln in toluene, 7.4 mmol, Aldrich). Toluene (190 mL) was added and the reaction mixture was cooled in an ice-salt bath (bath temp.=−10° C.). BH₃—SMe₂ was added (17 mL, 180 mmol, Aldrich), followed by a solution of 5-oxo-5,6,7,8-tetrahydronaphthalene-2-carboxylic acid methyl ester (30 g, 150 mmol, Albany Molecular) in THF (200 mL) was added over 5 h using a syringe pump. After the addition was complete, the reaction mixture was stirred for an additional 1 h. The reaction mixture was poured into an addition funnel, and the reaction mixture was added to MeOH (200 mL), cooled in a ice-salt bath, over 30 min at such a rate that the internal temp. was kept below 0° C. The reaction mixture was concentrated in vacuo. Et₂O (1 L) was added, and the mixture was washed with 1M H₃PO₄ (3×), satd NaHCO₃, and brine (ca. 400 mL each wash). The organic layer was dried over MgSO₄, filtered and concentrated in vacuo. The residue was dissolved in Et₂O again (500 mL), and the mixture was washed with 1M H₃PO₄ (3×200 mL), satd NaHCO₃, and brine. After drying the organic layer over MgSO₄, the mixture was filtered and concentrated in vacuo, which gave the title compound as a white-yellow solid. MS (+ ion ESI) m/z=207 (MH⁺), 189 (MH⁺—H₂O).

### Step B: Synthesis of 5(R)-azido-5,6,7,8-tetrahydronaphthalene-2-carboxylic acid methyl ester

To a 500 mL three-neck round-bottomed flask equipped with argon inlet/outlet, thermometer, and magnetic stirring was added 5(S)-hydroxy-5,6,7,8-tetrahydro-naphthalene-2-carboxylic acid methyl ester (29 g, 140 mmol) in toluene (280 mL). The reaction mixture was cooled in a ice-salt bath, and DPPA (36 mL, 170 mmol, Aldrich) was added (internal temp.=−4° C.). DBU (25 mL, 170 mmol, Aldrich) was added over 10 min at such a rate that the internal temp. of the reaction was kept below 1° C. The ice in the bath was allowed to melt, and the reaction continued for 12 h during which time the reaction mixture stopped stirring because a precipitate had formed. Stirring was resumed, and the reaction mixture was stirred at RT for another 11 h. The reaction contents were poured into a 2 L sep funnel, and the lower dark-brown layer was removed. Water (250 mL) was added to the remaining top layer, and the reaction mixture was extracted with Et₂O (3×250 mL). The combined organic layers were washed with 1M H₃PO₄, water, satd NaHCO₃, and brine. The organic layer was dried over MgSO₄, filtered and concentrated in vacuo. Purification by silica gel chromatography (330 g Isco Redisep® column, 1:1 hexane-CH₂Cl₂) of the crude material provided the title compound. MS (+ ion ESI) m/z=232 (MH⁺).

### Step C: Synthesis (5(R)-amino-5,6,7,8-tetrahydronaphthalen-2-yl)methanol

To an oven-dried, 3-neck, 2 L round-bottomed flask equipped with argon inlet/outlet, addition funnel, thermometer, and overhead stirring was added THF (700 mL) and LAH (470 mL of a 1M soln in THF, 470 mmol, Aldrich). The reaction mixture was cooled in a ice-salt bath, and 5-azido-5,6,7,8-tetrahydronaphthalene-2-carboxylic acid methyl ester (27 g, 120 mmol) in THF (100 mL) was added over ca. 30 min. The reaction mixture was warmed to RT overnight, then cooled in an ice-salt bath the next morning. Water (18 mL) in THF (20 mL) was added to the reaction mixture over 4 h. Vigorous gas evolution occurred. 5N NaOH (18 mL) was added over 30 min followed by water (54 mL). After stirring for an additional 1 h, the reaction mixture was filtered, and the filtrate was concentrated in vacuo. The residue was reconstituted in MeOH and CH₃CN, and concentrated in vacuo again to provide the title compound as light-brown solid. MS (+ ion ESI) m/z=161 (M-NH₃).

Similarly (4-(R)-aminochroman-7-yl)-methanol and (1-(R)-aminoindan-5-yl)methanol were prepared.

### Reference 2

### Synthesis of (R)-6-(1-piperidin-1-ylmethylvinyl)-1,2,3,4-tetrahydronaphthalen-1-ylamine

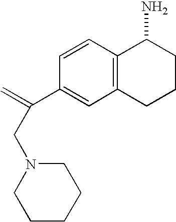

### Step A: Synthesis of trifluoromethanesulfonic acid 5-oxo-5,6,7,8-tetrahydronaphthalen-2-yl ester

To a 1-L round-bottomed flask charged with 6-hydroxy-1-tetralone (Aldrich, 21.97 g, 0.136 mol) at 0° C. was added CH₂Cl₂ (500 mL) and pyridine (Aldrich, 11 mL, 0.136 mol). Triflic anhydride (Aldrich, 23 mL, 0.136 mol) was added through an additional funnel over a period of 12 min. The reaction mixture was gradually warmed to room temperature and stirred at room temperature overnight. The residue was diluted with water and the two phases were separated. The organic phase was washed with 1N HCl (100 mL×2), saturated NaHCO₃, and brine, dried over Na₂SO₄. After filtration and concentration in vacuo, the crude was purified by flash chromatography (5-11% EtOAc-hexane) to provide the title product as yellow oil. MS (ESI): 295 (M+H)⁺.

### Step B: Synthesis of trifluoromethanesulfonic acid 5-hydroxy-5,6,7,8-tetrahydronaphthalen-2-yl ester

To a dry three-necked flask containing (R)-2-methyl-CBS-oxazaborolidine (Aldrich, 1.94 mL, 1.0M in toluene, 1.93 mmol, 0.05 eq) under N₂ was added a solution of borane-methylsulfide (BMS) (Aldrich, 3.30 mL, 34.80 mmol, 0.9 eq) in toluene (200 mL) through an additional funnel. After the addition was complete, the reaction mixture was allowed to cool to 0° C. A solution of trifluoromethanesulfonic acid 5-oxo-5,6,7,8-tetrahydro-naphthalen-2-yl ester (11.37 g, 38.67 mmol, 1.0 eq) in THF (180 mL) was added dropwise through an additional funnel. Following the addition, the reaction mixture was stirred at RT for additional 40 min, quenched with MeOH. The solvent was removed in vacuo and the crude was diluted with H₂O (50 mL). The aqueous phase was extracted with ether (3×150 mL). The combined organic layer was washed with brine, dried over Na₂SO₄, filtered and concentrated. The title compound was obtained as an off-white solid by flash chromatography (16-22% EtOAc-hexane).

### Step C: Synthesis of trifluoromethanesulfonic acid 5-azido-5,6,7,8-tetrahydronaphthalen-2-yl ester

To a solution of trifluoromethanesulfonic acid 5-hydroxy-5,6,7,8-tetrahydronaphthalen-2-yl ester (11.2 g, 37.9 mmol, 1.0 eq) in THF (150 mL) at RT was added DPPA (Aldrich, 11.1 mL, 51.6 mmol, 1.36 eq). The resulting mixture was allowed to cool to 0° C. and DBU (Aldrich, 7.7 mL, 51.6 mmol, 1.36 eq) was added slowly through a syringe. The reaction mixture was allowed to warm to RT and stirred over the weekend. The reaction mixture was concentrated in vacuo. The crude product was dissolved in EtOAc (400 mL). The organic layer was washed with NH₄Cl (twice), H₂O, and brine, dried over Na₂SO₄. After filtration and concentration in vacuo, the crude was purified by flash chromatography (5% EtOAc-hexane) to provide the title compound.

### Step D: Synthesis of trifluoromethanesulfonic acid 5-amino-5,6,7,8-tetrahydronaphthalen-2-yl ester

A solution of trifluoromethanesulfonic acid 5-azido-5,6,7,8-tetrahydronaphthalen-2-yl ester (10.3 g, 32.1 mmol, 1.0 eq) in THF (70 mL) was added PPh₃ (Aldrich, 8.4 g, 32.1 mmol, 1.0 eq), and H₂O (30 mL) at 0° C. The reaction mixture was allowed to warmed to RT and stirred overnight. 2N HCl was added until the mixture was acidic (PH=1-2). The mixture was extracted with toluene (3×100 mL). The aqueous phase was neutralized with 5N NaOH until the pH is 12-13, and the product was extracted with ether (3×150 mL). The ether solution was dried over Na₂SO₄, filtered and concentrated in vacuo. The crude was purified by flash chromatography (6% MeOH—CH₂Cl₂) to provide the title compound.

### Step E: Synthesis of trifluoromethanesulfonic acid 5-tert-butoxycarbonylamino-5,6,7,8-tetrahydro-naphthalen-2-yl ester

A solution of trifluoromethanesulfonic acid 5-amino-5,6,7,8-tetrahydronaphthalen-2-yl ester (2.0 g, 6.8 mmol, 1.0 eq) in CH₂Cl₂ (20 mL) was added Et₃N (1.9 mL, 13.6 mmol, 2.0 eq) and di-tert-butyl carbonate (Aldrich, 1.8 g, 8.1 mmol, 1.2 eq). The reaction mixture was stirred at RT overnight, washed with saturated NaHCO₃ (2×20 mL), brine, dried over Na₂SO₄. After filtration and concentration in vacuo, the crude was purified by flash chromatography (4-10% EtOAc-hexane) to provide the title compound as a white solid.

### Step F: Synthesis of [6-(1-piperidin-1-ylmethylvinyl)-1,2,3,4-tetrahydronaphthalen-1-yl]-carbamic acid tert-butyl ester

A solution of trifluoromethanesulfonic acid 5-tert-butoxycarbonylamino-5,6,7,8-tetrahydronaphthalen-2-yl ester (1.89 g, 4.79 mmol, 1.0 eq) in CH₃CN (25 mL) purged with N₂ was added palladium (II) acetate (Strem Chemicals, 65 mg, 0.29 mmol, 0.06 eq), 1,1′-bis(diphenylphosphino)ferrocene (Aldrich, 0.70 g, 1.26 mmol, 0.26 eq), K₂CO₃ (0.99 g, 7.18 mmol, 1.5 eq) and N-allylpiperidine (Lancaster, 3.00 g, 23.96 mmol, 5.0 eq). The reaction mixture sealed with a septum was heated to 80° C. overnight, cooled to RT, diluted with H₂O, and extracted with ether. The ether solution was dried over Na₂SO₄, filtered and concentrated in vacuo. The crude was purified by flash chromatography (14-21% EtOAc-Hexane) to provide the title compound. MS (ESI): 371 (M+H)⁺.

### Step G: Synthesis of 6-(1-piperidin-1-ylmethylvinyl)-1,2,3,4-tetrahydronaphthalen-1-ylamine

To a solution of [6-(1-piperidin-1-ylmethylvinyl)-1,2,3,4-tetrahydronaphthalen-1-yl]-carbamic acid tert-butyl ester in CH₂Cl₂ (3 mL) was added TFA (3 mL). The reaction mixture was stirred at RT for 4 h and then concentrated in vacuo. The crude was neutralized with 10% Na₂CO₃ until the aqueous phase is basic, extracted with CH₂Cl₂. The organic solution was washed with brine, dried over Na₂SO₄, filtered and concentrated in vacuo to provide the title compound. MS (ESI): 271 (M+H)⁺.

### Reference 3

### Synthesis of (R)-6-((tert-butylamino)methyl)-1,2,3,4-tetrahydronaphthalen-1-ylamine

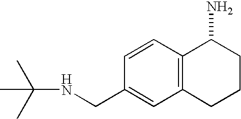

### Step A: Synthesis of (R)-tert-butyl 6-(hydroxymethyl)-1,2,3,4-tetrahydronaphthalen-1-ylcarbamate

Triethylamine (27.7 mL, 199 mmol) and di-tert-butyl-dicarbonate (17.4 g, 79.6 mmol) were added consecutively to a solution of (5(R)-amino-5,6,7,8-tetrahydronaphthalen-2-yl)-methanol (7.05 g, 39.8 mmol) in a mixed solvent of ethyl acetate (100 mL), methanol (100 mL), and dichloromethane (100 mL) and the reaction mixture was stirred at RT for 2 h. The solvents were removed in vacuo, the residue was partitioned between ethyl acetate and saturated sodium bicarbonate. The organic portion was separated, washed with brine, the solvents were removed to afford a white solid, used in the following reaction without purification.

### Step B: Synthesis of (R)-tert-butyl 6-formyl-1,2,3,4-tetrahydronaphthalen-1-ylcarbamate

A mixture of (R)-tert-butyl 6-(hydroxymethyl)-1,2,3,4-tetrahydronaphthalen-1-yl-carbamate (11.0 g, 39.8 mmol) and MnO₂ (Aldrich, <5 micron, 85%, 20.3 g, 198 mmol) in dichloromethane (500 mL) was stirred at r.t. overnight, filtered through a pad of Celite and the solvents were removed to afford a light yellowish viscous oil which was used in the following reaction without purification.

### Step C: Synthesis of (R)-tert-butyl 6-((tert-butylamino)methyl)-1,2,3,4-tetrahydronaphthalen-1-ylcarbamate

A mixture of (R)-tert-butyl 6-formyl-1,2,3,4-tetrahydronaphthalen-1-ylcarbamate (39.8 mmol), tert-butylamine (21.0 mL, 199 mmol), and acetic acid (2.4 mL, 36.0 mmol) in of DMF (50 mL) in a sealed vessel was stirred at 60° C. for 1 h, cooled to r.t. and diluted with MeOH. NaBH₄ (3.0 g, 80 mmol) was added portion wise and the reaction mixture was stirred at RT for 10 min. MeOH was removed under reduced pressure and the DMF solution was partitioned between ethyl acetate and water. The organic portion was separated and the solvents were removed to afford a viscous oil which was used in the following reaction without purification.

### Step D: Synthesis of (R)-6-((tert-butylamino)methyl)-1,2,3,4-tetrahydronaphthalen-1-ylamine

A solution of (R)-tert-butyl 6-((tert-butylamino)methyl)-1,2,3,4-tetrahydronaphthalen-1-ylcarbamate (13.3 g, 39.8 mmol) in 300 mL of a saturated hydrogen chloride solution in ethyl acetate was stirred at RT overnight in a sealed flask and the solids were collected by filtration.

Similarly, (R)-7-((tert-butylamino)methyl)chroman-4-amine and (R)-5-((tert-butylamino)methyl)-2,3-dihydro-1H-inden-1-amine were prepared.

### Reference 4

### Synthesis of (R)-6-(piperidin-1-ylmethyl)-1,2,3,4-tetrahydronaphthalen-1-ylamine

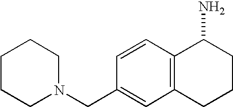

### Step A: Synthesis of (R)-tert-butyl 6-(piperidin-1-ylmethyl)-1,2,3,4-tetrahydronaphthalen-1-ylcarbamate

(R)-tert-Butyl 6-formyl-1,2,3,4-tetrahydronaphthalen-1-ylcarbamate (12.2 g, 44.33 mmol) and piperidine (22 mL, 221.6 mmol, 5.0 equiv) were dissolved in 1,2-dichloroethane (400 mL). Acetic acid (10 drops) and sodium triacetoxyborohydride (23.4 g, 110.82 mmol, 2.5 equiv) were added and the reaction mixture was heated at 50° C. for 15 h. The reaction mixture was cooled to room temperature, diluted with EtOAc (600 mL), washed with saturated sodium bicarbonate solution (2×250 mL), saturated ammonium chloride solution (200 mL), brine (200 mL), dried (MgSO₄) and purified on silica gel using 5% methanol in dichloromethane as eluant, affording (R)-tert-butyl-6-(piperidin-1-ylmethyl)-1,2,3,4-tetrahydronaphthalen-1-ylcarbamate. MS: 345.2 (M+H).

### Step B: Synthesis of (R)-6-(piperidin-1-ylmethyl)-1,2,3,4-tetrahydronaphthalen-1-amine

A solution of (R)-tert-butyl 6-(piperidin-1-ylmethyl)-1,2,3,4-tetrahydronaphthalen-1-ylcarbamate (15.0 g, 43.57 mmol) in methanol (325 mL) was treated with a 1.0M solution of HCl in diethyl ether (300 mL, 300 mmol, 6.8 equiv), capped and stirred at room temperature for 14 h. The reaction mixture was concentrated in vacuo and partioned in dichloromethane (300 mL) and 1.0N NaOH (300 mL). The aqueous layer was separated and extracted with dichloromethane (2×250 mL). The combined organic layers were washed with brine (200 mL), dried (MgSO₄) and concentrated, affording (R)-6-(piperidin-1-ylmethyl)-1,2,3,4-tetrahydronaphthalen-1-amine as a dark yellow oil. MS: 245.2 (M+H).

Similarly, (R)-7-(piperidin-1-ylmethyl)chroman-4-amine, (R)-5-(piperidin-1-ylmethyl)-2,3-dihydro-1H-inden-1-amine and (R)-6-((4-methylpiperidin-1-yl)methyl)-1,2,3,4-tetrahydronaphthalen-1-amine were prepared.

### Reference 5

### Synthesis of 7-(tert-butylaminomethyl)-6-chloro-chroman-(4R)-ylamine

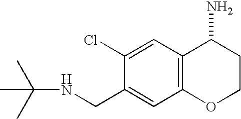

### Step A: Synthesis of 7-bromomethyl-6-chlorochroman-4-one

A mixture of 6-chloro-7-methylchroman-4-one (20 g, 102 mmol), NBS (19.9 g, 112 mmol), and AIBN (4.17 g, 25.4 mmol) in anhydrous CCl₄ (300 mL) was heated at reflux for 24 h. The reaction mixture was cooled and the solid was filtered off. The filtrate was concentrated and used in the next step without purification.

### Step B: Synthesis of 7-(tert-butylamino-methyl)-6-chloro-chroman-4-one

To a stirred mixture of tert-butylamine (7.3 g, 100. Mmol) and Et₃N (10.1 g, 99.8 mmol) in anhydrous CH₂Cl₂ (50 ml) was added a solution of 7-bromomethyl-6-chloro-chroman-4-one (25 g, 91 mmol) in CH₂Cl₂ (150 ml) dropwise. Stirring was continued for 16 h after which the mixture was concentrated, taken up in H₂O, acidified with 10% HCl until ph 1, and extracted with Et₂O (discarded). The acidic aqueous layer was neutralized with 5N NaOH, and extracted with CH₂Cl₂ (3×). The combined extracts were dried over MgSO₄, concentrated to give a yellow solid.

### Step C: Synthesis of 7-(tert-butylamino-methyl)-6-chloro-chroman-(4S)-ol

To a stirred solution of (1S,2S)-(+)-N-(4-toluene-sulfonyl)-1,2-diphenylethylenediamine (0.29 g, 8.1 mmol) in i-PrOH (15 ml) was added [RuCl₂(N-6-p-cymene)]₂, and Et₃N under argon. The reaction mixture was heated at 80° C. for 1 h, cooled, and concentrated to dryness. To this mixture was added a solution of 7-(tert-butylamino-methyl)-6-chloro-chroman-4-one (12 g, 45 mol) in anhydrous CH₃CN (150 ml), followed by 5:2 formic acid/TEA (6 ml). The reaction was stirred at rt for 24 h and then concentrated, taken up in H₂O, neutralized with 10% Na₂CO₃, extracted with CH₂Cl₂ (3×), dried over MgSO₄, concentrated to give a brown foam which was stirred in hexane/ether (1:1), and filtered. The filtrate was concentrated to give a light brown foam.

### Step D: Synthesis of 4(R)-azido-6-chloro-chroman-7-ylmethyl)tert-butylamine

To a stirred, cooled (0° C.) solution of 7-(tert-butylaminomethyl)-6-chlorochroman-(4S)-ol (11.55 g, 42.91 mmol) in anhydrous toluene (150 mL) was added DPPA (23.6 g, 85.8 mmol) dropwise in 0.5 h and DBU (13.1 g, 85.9 mmol). The reaction mixture was stirred at rt for 24 h. The reaction mixture was concentrated, taken up in H₂O, extracted with CH₂Cl₂ (3×), dried over MgSO₄, concentrated and purified by ISCO (3% MeOH/CH₂Cl₂) to give a brown oil. MS (APCI) m/z 296 (M+2).

### Step E: Synthesis of 7-(tert-butylaminomethyl)-6-chlorochroman-(4R)-ylamine

A mixture of (4R)-azido-6-chlorochroman-7-ylmethyl)tert-butylamine (12 g, 41 mmol) and Ph₃P (16 g, 61 mmol) in anhydrous THF (100 mL) was stirred at RT in 3 h. H₂O (100 mL) was added and the reaction mixture was heated at reflux for 24 h. The reaction mixture was cooled, concentrated, taken up in toluene, extracted with 5N HCl. The aqueous layer was neutralized with 10N NaOH, extracted with CHCl₃ (3×), dried over MgSO₄, concentrated to give a brown oil. MS (APCI) m/z 270 (M+2).

Similarly, (R)-6-chloro-7-(piperidin-1-ylmethyl)chroman-4-amine was prepared.

### Reference 6

### Synthesis of (R)-6-(2-(piperidin-1-yl)ethyl)-1,2,3,4-tetrahydronaphthalen-1-amine

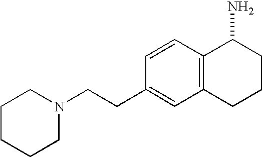

### Step A: Synthesis of (R)-tert-butyl 6-(iodomethyl)-1,2,3,4-tetrahydronaphthalen-1-ylcarbamate

To a solution of (R)-tert-butyl 6-(hydroxymethyl)-1,2,3,4-tetrahydronaphthalen-1-ylcarbamate (415.5 mg, 1.5 mmol) in dichloromethane/ether (1:1, 30 mL) at room temperature were added triphenylphosphine (590 mg, 2.25 mmol) and imidazole (153 mg, 2.25 mmol). To this stirred solution was then added iodine (571 mg, 2.25 mmol). After stirring for 20 min, the reaction was quenched with 10% Na₂S₂O₃ (15 mL) until it became a clear two-phase solution. The aqueous phase was extracted with ether. The combined organic phase was dried over Na₂SO₄, filtered, and evaporated to dryness. Flash chromatography (SiO₂, hexane/CH₂Cl₂=3:1 to pure CH₂Cl₂) afforded the desired product as a white solid.

### Step B: Synthesis of (R)-tert-butyl 6-((1,3-dithian-2-yl)methyl)-1,2,3,4-tetrahydronaphthylen-1-ylcarbamate

To a solution of 1,3-dithiane (1.01 g, 8.4 mmol) in 10 mL of dry THF at −30° C. was added dropwise 2.5M n-butyllithium in hexane (3.36 mL, 8.4 mmol). After stirring at −20° C. for 1.5 h, a solution of the iodide obtained in Step A (542 mg, 1.4 mmole, azeotroped with benzene) in dry THF (10 mL) was added dropwise at −20° C. The reaction mixture was stirred at −5° C. to 0° C. for 1 h. The reaction mixture was quenched with sat. NH₄Cl solution, extracted with EtOAc, dried over Na₂SO₄, filtered, and evaporated to dryness. Flash chromatography (SiO₂, CH₂Cl₂/hexane=1:1 to 2:1 to CH₂Cl₂/EtOAc=100:3) afforded the title compound as a white solid.

### Step C: Synthesis of (R)-tert-butyl 6-(2-oxoethyl)-1,2,3,4-tetrahydronaphthalen-1-ylcarbamate

(R)-tert-butyl 6-((1,3-dithian-2-yl)methyl)-1,2,3,4-tetrahydronaphthylen-1-ylcarbamate

(6.1 g, 16.1 mmol) and CaCO₃ (3.23 g, 32.3 mmol) were suspended in THF/water (120 mL, 5:1 ratio) and Hg(ClO₄)₂ (9.65 g, 24.15 mmol) was added portion wise. After stirring at room temperature for 2 h, the reaction mixture was filtered through a celite pad with the help of EtOAc. The filtrate was evaporated to dryness and the residue was purified by flash chromatography (SiO₂, DCM to DCM/EtOAc=3:1 to 2:1) gave the title compound as a colorless sticky oil.

### Step D: Synthesis of (R)-tert-butyl-6-(2-(piperidin-1-yl)ethyl)-1,2,3,4-tetrahydronaphthalen-1-ylcarbamate

To a solution of (R)-tert-butyl 6-(2-oxoethyl)-1,2,3,4-tetrahydronaphthalen-1-yl-carbamate (2.02 g, 7 mmol) and pyperidine (1.79 g, 21 mmol) in dichloroethane (10 mL) was added sodium triacetoxyborohydride (2.97 g, 14 mmol). After stirring overnight at room temperature, the reaction solution was diluted with EtOAc and washed with sat. NaHCO₃ and brine. The organic phase was dried over Na₂SO₄ and evaporated to dryness in vacuo. The crude was purified by flash chromatography (SiO₂, DCM to EtOAc to EtOAc/MeOH=100:20) to give the title compound as a sticky oil.

### Step E: Synthesis of (R)-6-(2-(piperidin-1-yl)ethyl)-1,2,3,4-tetrahydronaphthalen-1-amine

To a solution of (R)-tert-butyl-6-(2-(piperidin-1-yl)ethyl)-1,2,3,4-tetrahydronaphthalen-1-ylcarbamate (2.0 g, 5.35 mmol) in DCM (25 mL) at room temperature was added TFA (3.66 g, 32 mmol). After stirring at room temperature overnight, the reaction mixture was evaporated to dryness. The residue was treated with 2.5 mL of triethylamine and it was evaporated again in vacuo. The crude product was azeotroped with benzene and was directly used in the next step.

### Reference 7

### Synthesis of 2-(5(R/S)-amino-5,6,7,8-tetrahydroquinazolin-2-yl)ethanol

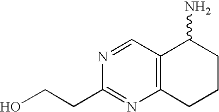

### Step A: Synthesis of 3-(tert-butyldiphenylsilyloxy)-propanenitrile

To a solution of 3-hydroxypropanenitrile (7.1 g, 0.1 mol) and DMAP (1.22 g, 0.01 mmol) in dry DCM (30 mL) at room temperature was added Et₃N (30.3 g, 0.3 mol), followed by TBDPSCl (27.5 g, 0.1 mol). A lot of white solid were formed. After stirring at room temperature overnight, the reaction mixture was quenched with sat. NH₄Cl solution, extracted with DCM, dried over Na₂SO₄, and evaporated in vaco. Flash chromatography (SiO₂, hexane/EtOAc=100:2 to 100:5 to 100:10) of the crude gave of 3-(tert-butyldiphenylsilyloxy)-propanenitrile as a white solid.

### Step B: Synthesis of 3-(tert-butyldiphenylsilyloxy)-propanamidine

To a suspension of NH₄Cl (5.35 g, 0.1 mol) in dry benzene (60 mL) at 0° C. was slowly added a solution of trimethylaluminum in toluene (50 mL, 2M). After the addition was complete, the reaction mixture was allowed to warm up to room temperature and was stirred for 2 h until gas evolution had ceased. A solution of 3-(tert-butyldiphenylsilyloxy)propanenitrile (9.27 g, 0.03 mol) in dry benzene (20 mL) was added to the aluminum amide reagent and the resulting mixture was heated up to 80° C. for 20 h. The reaction mixture was slowly cooled to room temperature and then carefully poured into a slurry of 300 mL of DCM and 200 g of silica gel. It was then filtered and washed thoroughly with MeOH/DCM (1:2). After concentration, flash chromatography (SiO₂, EtOAc to EtOAc/MeOH=100:20 to 100:30 to EtOAc/2M NH₃ in MeOH=100:30) gave the title compound as a white solid.

### Step C: Synthesis of 2-(2-(tert-butyldiphenylsilyloxy)ethyl)-7,8-dihydroquinazolin-5(6H)-one

A solution of 3-(tert-butyldiphenylsilyloxy)-propanamidine (25 g, 77 mmol) and 2-((dimethylamino)methylene)cyclohexane-1,3-dione (12.8 g, 77 mmol) in dry EtOH (400 mL) was heated at 80° C. for 3 h. After cooling to room temperature, the solvent was evaporated. Flash chromatography (SiO₂, EtOAc/hexane=1:1) gave the title compound as a white solid.

### Step D: Synthesis of 2-(2-(tert-butyldiphenylsilyloxy)ethyl)-5,6,7,8-tetrahydro-quinazolin-5-ol

A solution of 2-(2-(tert-butyldiphenylsilyloxy)ethyl)-7,8-dihydroquinazolin-5(6H)-one (2.16 g, 5 mmol) in dry MeOH (30 mL) was treated with NaBH₄ (189 mg, 5 mmol). After 5 min, the reaction was quenched with 5 mL of sat. NH₄Cl solution. The MeOH was evaporated and the residue was extracted with DCM, dried over Na₂SO₄ and evaporated. Flash chromatography (SiO₂, DCM to EtOAc) gave the title compound as a white solid.

### Step E: Synthesis of 5-azido-2-(2-(tert-butyldiphenylsilyloxy)ethyl)-5,6,7,8-tetrahydroquinazoline

To a solution of 2-(2-(tert-butyldiphenylsilyloxy)ethyl)-5,6,7,8-tetrahydroquinazolin-5-ol (2.0 g, 4.63 mmol) in toluene (25 mL) at −10° C. was added DPPA (1.2 mL, 5.56 mmol). To this stirred solution was then added DBU (0.83 mL, 5.56 mmol) dropwise while keeping the temperature below 0° C. After stirring at room temperature for 16 h, the reaction was evaporated to dryness and directly submitted to flash chromatography (SiO₂, hexane/DCM=1:2) to afford the title compound as a white solid.

### Step F: Synthesis of 2-(2-(tert-butyldiphenylsilyloxy)ethyl)-5,6,7,8-tetrahydro-quinazolin-5-amine

A suspension of Pd/C (80 mg, 10% w/w) in a solution of 5-azido-2-(2-(tert-butyldiphenylsilyloxy)ethyl)-5,6,7,8-tetrahydroquinazoline (800 mg, 1.75 mmol) in EtOAc (30 mL) was stirred under H₂ atomosphere overnight. The reaction mixture was then directly submitted to flash chromatograph (SiO₂, EtOAc to EtOAc/MeOH=100:15 to EtOAc/2M NH₃ in MeOH=2:1) to give the title compound as a white solid.

### Step G: Synthesis of 2-(5-amino-5,6,7,8-tetrahydroquinazolin-2-yl)ethanol

A solution of 2-(2-(tert-butyldiphenylsilyloxy)ethyl)-5,6,7,8-tetrahydroquinazolin-5-amine (570 mg, 1.32 mmol) in THF (10 mL) at 0° C. was treated with a 1M TBAF solution in THF (1.56 mL, 1.56 mmol). After stirring at room temperature overnight, the reaction mixture was directly submitted to flash chromatograph (SiO₂, EtOAc to EtOAc/MeOH=100:15 to EtOAc/2M NH₃ in MeOH=1:1) to give crude product as a white solid.

### Reference 8

### Synthesis of 1-(2-(tert-butyldimethylsilyloxy)ethyl)-4,5,6,7-tetrahydro-1H-indazol-4-ylamine

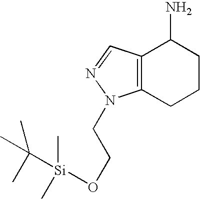

### Step A: Synthesis of 1-(2-hydroxyethyl)-6,7-dihydro-1H-indazol-4(5H)-one

2-Hydroxyethyl hydrazine (1.36 mL, 20 mmol) was slowly added to an ice-cooled solution of 2-((dimethylamino)methylene)cyclohexane-1,3-dione (3.34 g) in methanol (50 mL). After stirring the reaction mixture at room temperature for 20 min, the solvent was evaporated. Flash chromatography (SiO₂, EtOAc/MeOH=100:5 to 100:7 to 100:10) on the crude product gave the title compound as a white solid.

### Step B: Synthesis of 1-(2-(tert-butyldimethylsilyloxy)ethyl)-6,7-dihydro-1H-indazol-4(5H)-one

To a solution of 1-(2-hydroxyethyl)-6,7-dihydro-1H-indazol-4(5H)-one (14 g, 77.8 mmol) in dry DCM (100 mL) was added Et₃N (22 mL, 155.6 mmol), followed by TBSCl (14 g, 93.3 mmol) and DMAP (95 mg, 0.78 mmol). After stirring at room temperature overnight, the reaction mixture was quenched with brine and extracted with EtOAc. Flash chromatography (SiO₂, EtOAc/hexane=1:1) of the crude product gave the title compound as a white solid.

### Step C: Synthesis of 1-(2-(tert-butyldimethylsilyloxy)ethyl)-4,5,6,7-tetrahydro-1H-indazol-4-ol

A solution of 1-(2-(tert-butyldimethylsilyloxy)ethyl)-6,7-dihydro-1H-indazol-4(5H)-one (21 g, 71.4 mmol) in dry MeOH (200 mL) was treated with NaBH₄ (2.7 g, 71.4 mmol). After 30 min, the reaction mixture was quenched with 15 mL of sat. NH₄Cl solution. The MeOH was evaporated and the residue was extracted with EtOAc, dried over Na₂SO₄ and evaporated. Flash chromatography (SiO₂, EtOAc/hexane=1:1 to EtOAc) of the crude product gave the title compound as a white solid.

### Step D: Synthesis of 4-azido-1-(2-(tert-butyldimethyl silyloxy)ethyl)-4,5,6,7-tetrahydro-1H-indazole

To a solution of 1-(2-(tert-butyldimethylsilyloxy)ethyl)-4,5,6,7-tetrahydro-1H-indazol-4-ol (23 g, 77.7 mmol) in 200 mL of toluene at −10° C. was added DPPA (20 mL, 93.2 mmol). DBU (13.9 mL, 93.2 mmol) was added dropwise while keeping the temperature below 0° C. After stirring at room temperature for 18 h, the reaction mixture was evaporated to dryness and directly submitted to flash chromatography (SiO₂, hexane/EtOAc=2:1 to EtOAc) to afford the title compound as a colorless liquid, together with 12 g of recovered starting alcohol.

### Step E: Synthesis of 1-(2-(tert-butyldimethylsilyloxy)ethyl)-4,5,6,7-tetrahydro-1H-indazol-4-amine

A suspension of 150 mg of Pd/C (10% w/w) in a solution of 4-azido-1-(2-(tert-butyldimethyl silyloxy)ethyl)-4,5,6,7-tetrahydro-1H-indazole (2.0 g, 6.23 mmol) in EtOAc (100 mL) was stirred under H₂ atomosphere overnight. The reaction mixture was then directly submitted to flash chromatograph (SiO₂, EtOAc to EtOAc/MeOH=100:20 to EtOAc/2M NH₃ in MeOH=100:20 to 100:30 to 100:40) to give the title compound as a white solid.

### Reference 9

### Synthesis of (R)-7-nitro-6-(piperidin-1-ylmethyl)-1,2,3,4-tetrahydronaphthalen-1-ylamine

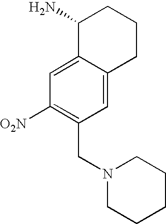

### Step A: Synthesis of (R)-2,2,2-trifluoro-N-(6-(hydroxymethyl)-1,2,3,4-tetrahydronaphthalen-1-yl)acetamide

To a solution of (R)-(5-amino-5,6,7,8-tetrahydronaphthalen-2-yl)methanol (5.32 g, 30 mmol) and trfluoroacetic anhydride (4.66 mL, 33 mL) in 20 mL of dry THF at RT was added dropwise triethylamine (5.0 mL, 36 mmol). After stirring at RT for 2 h, the reaction was quenched with sat. NaHCO₃, extracted with EtOAc, washed with brine, dried over Na₂SO₄, filtered, and evaporated to dryness. Flash chromatography (SiO₂, DCM/EtOAc=1:1 to pure EtOAc) afforded the title compound as a white solid.

### Step B: Synthesis of (R)-(5-(2,2,2-trifluoroacetamido)-5,6,7,8-tetrahydronaphthalen-2-yl)methylacetate

To a solution of (R)-2,2,2-trifluoro-N-(6-(hydroxymethyl)-1,2,3,4-tetrahydronaphthalen-1-yl)acetamide (546 mg, 2 mmol) in 10 mL of dry DCM was added acetic anhydride (0.30 mL, 3 mmol) and triethylamine (0.8 mL, 6 mmol), followed by DMAP (10 mg). After stirring for 2 h at RT, the solution was evaporated to dryness and directly submitted to flash chromatography (SiO₂, DCM) to give the title compound as a white solid.

### Step C: Synthesis of (R)-(1-nitro-5-(2,2,2-trifluoroacetamido)-5,6,7,8-tetrahydronaphthalen-2-yl)methyl acetate and (R)-(3-nitro-5-(2,2,2-trifluoroacetamido)-5,6,7,8-tetrahydronaphthalen-2-yl)methyl acetate

To a solution of (R)-(5-(2,2,2-trifluoroacetamido)-5,6,7,8-tetrahydronaphthalen-2-yl)methyl acetate (160 mg, 0.508 mmol) in MeCN (2 mL) was added NO₂⁺BF₄⁻ (66 mg, 0.508 mmol). After stirring at RT for 20 min, the reaction was quenched with 0.5 mL of sat. NaHCO₃, and the solvent was evaporated to dryness. Flash chromatography (SiO₂, DCM/hexane=5:1 to pure DCM) gave (R)-(1-nitro-5-(2,2,2-trifluoroacetamido)-5,6,7,8-tetrahydronaphthalen-2-yl)methyl acetate (40 mg) as a white solid and (R)-(3-nitro-5-(2,2,2-trifluoroacetamido)-5,6,7,8-tetrahydronaphthalen-2-yl)methyl acetate as a white solid.

### Step D: Synthesis of (R)-2,2,2-trifluoro-N-(6-(hydroxymethyl)-7-nitro-1,2,3,4-tetrahydronaphthalen-1-yl)acetamide

To a solution of (R)-(3-nitro-5-(2,2,2-trifluoroacetamido)-5,6,7,8-tetrahydronaphthalen-2-yl)methyl acetate (17 mg) in 10 mL of MeOH was added one drop of 96% H₂SO₄ and the resulting mixture was stirred at 50° C. for 3 h. After cooling to RT, the reaction was quenched with sat. NaHCO₃ (0.5 mL). The MeOH was evaporated and the residue was directly loaded on column chromatography (SiO₂, DCM to DCM/EtOAc=2:1 to 1:1) to give the title compound as a white solid.

### Step E: Synthesis of (R)-2,2,2-trifluoro-N-(6-formyl-7-nitro-1,2,3,4-tetrahydronaphthalen-1-yl)acetamide

To a solution of (R)-2,2,2-trifluoro-N-(6-(hydroxymethyl)-7-nitro-1,2,3,4-tetrahydronaphthalen-1-yl)acetamide (350 mg, 1.1 mmol) in DCM (100 mL) was added portion wise MnO₂ (957 mg, 11 mmol). After stirring at RT for 3 h, the reaction mixture was filtered through silica gel with the help of hexane/EtOAc=1:1 to give the title compound as a white solid.

### Step F: Synthesis of (R)-2,2,2-trifluoro-N-(7-nitro-6-(piperidin-1-ylmethyl)-1,2,3,4-tetrahydronaphthalen-1-yl)acetamide

To a solution of (R)-2,2,2-trifluoro-N-(6-formyl-7-nitro-1,2,3,4-tetrahydronaphthalen-1-yl)acetamide (350 mg, 1.1 mmol) in 1,2-dichloroethane (6 mL) was added piperidine (187 mg, 2.2 mmol) and NaBH(OAc)₃ (350 mg, 1.65 mmol). After stirring at RT overnight, the reaction was quenched with sat. NaHCO₃, extracted with EtOAc, dried over Na₂SO₄, filtered, and evaporated to dryness. Flash chromatography (SiO₂, EtOAc/hexane=1:1) afforded the title compound as a white solid.

### Step G: Synthesis of (R)-7-nitro-6-(piperidin-1-ylmethyl)-1,2,3,4-tetrahydronaphthalen-1-amine

A solution of (R)-2,2,2-trifluoro-N-(7-nitro-6-(piperidin-1-ylmethyl)-1,2,3,4-tetrahydronaphthalen-1-yl)acetamide (230 mg, 0.6 mmol) and NaOH (108 mg, 2.7 mmol) in a mixed solvent (THF/MeOH/H₂O=5 mL/5 mL/1 mL) was heated at 70° C. for 3 h. After cooling to RT, the solvent was evaporated to dryness and the residue was directly loaded on column chromatography (SiO₂, EtOAc to EtOAc/2M NH₃ in MeOH=100:15 to 100:20) to give the title as a white solid.

### Reference 10

### Synthesis of (R)-5-nitro-6-(piperidin-1-ylmethyl)-1,2,3,4-tetrahydronaphthalen-1-ylamine

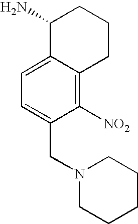

### Step A: Synthesis of (R)-2,2,2-trifluoro-N-(6-(hydroxymethyl)-5-nitro-1,2,3,4-tetrahydro-naphthalen-1-yl)acetamide

To a solution of (R)-(1-nitro-5-(2,2,2-trifluoroacetamido)-5,6,7,8-tetrahydronaphthalen-2-yl)methyl acetate (560 mg) in 10 mL of MeOH was added one drop of 96% H₂SO₄ and the resulting mixture was stirred at 50° C. for 3 h. After cooling to RT, the reaction was quenched with sat. NaHCO₃ (0.5 mL). The MeOH was evaporated and the residue was directly loaded on column chromatography (SiO₂, hexane/EtOAc=2:1) to give the title compound as a white solid.

### Step B: Synthesis of (R)-2,2,2-trifluoro-N-(6-formyl-5-nitro-1,2,3,4-tetrahydronaphthalen-1-yl)acetamide

To a solution of (R)-2,2,2-trifluoro-N-(6-(hydroxymethyl)-5-nitro-1,2,3,4-tetrahydronaphthalen-1-yl)acetamide (440 mg, 1.38 mmol) in DCM (50 mL) was added portionwise MnO₂ (1.20 g, 13.8 mmol). After stirring at RT for 1 h, the reaction mixture was filtered through silica gel with the help of hexane/EtOAc=1:1 to give the title compound as a white solid.

### Step C: Synthesis of (R)-2,2,2-trifluoro-N-(5-nitro-6-(piperidin-1-ylmethyl)-1,2,3,4-tetrahydronaphthalen-1-yl)acetamide

To a solution of (R)-2,2,2-trifluoro-N-(6-formyl-5-nitro-1,2,3,4-tetrahydronaphthalen-1-yl)acetamide (250 mg, 0.79 mmol) in 1,2-dichloroethane (2 mL) and 0.5 mL of HOAc was added piperidine (135 mg, 1.58 mmol) and NaBH(OAc)₃ (251 mg, 1.19 mmol). After stirring at RT overnight, the reaction was quenched with sat. NaHCO₃, extracted with EtOAc, dried over Na₂SO₄, filtered, and evaporated to dryness. Flash chromatography (SiO₂, EtOAc/hexane=1:4 to 1:3 to 1:2) afforded the title compound as a white solid.

### Step D: Synthesis of (R)-5-nitro-6-(piperidin-1-ylmethyl)-1,2,3,4-tetrahydronaphthalen-1-amine

A solution of (R)-2,2,2-trifluoro-N-(5-nitro-6-(piperidin-1-ylmethyl)-1,2,3,4-tetrahydronaphthalen-1-yl)acetamide (230 mg, 0.6 mmol) and NaOH (48 mg, 1.2 mmol) in a mixed solvent (MeOH/H₂O=10 mL/1 mL) was heated at 70° C. for 3 h. After cooling to RT, the solvent was evaporated to dryness and the residue was directly loaded on column chromatography (SiO₂, EtOAc to EtOAc/2M NH₃ in MeOH=100:10 to 100:20 to 100:30) to give the title compound as a white solid.

### Reference 11

### Synthesis of (R)-2-hydroxy-3,3-dimethyl-4-(naphthalen-2-ylsulfonyl)butanoic acid

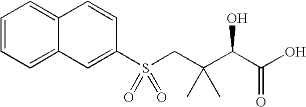

### Step A: Synthesis of (R)-2-hydroxy-3,3-dimethyl-4-(naphthalen-2-ylthio)butanoic acid

A mixture of naphthalene-2-thiol (1.3828 g, 8.6 mmol), (R)-3-hydroxy-4,4-dimethyl-dihydrofuran-2(3H)-one (1.0199 g, 7.8 mmol) and potassium carbonate (2.3851 g, 17 mmol) in DMF (10.0 mL) was stirred under microwave at 120° C. for 1 h, and then at 150° C. for 1 h. To the reaction mixture was added 1N HCl aq. (200 mL) and the product was extracted with AcOEt (100 mL×2). The combined organic phase was washed with 1N HCl aq. (200 mL) and sat'd NaCl aq. (100 mL×2), and dried over Na₂SO₄. The solvent was removed under reduced pressure and chromatographed on silica (Et₂O→Et₂O/AcOH=50/1). The semi-purified product was re-chromatographed on silica (CH₂Cl₂→CH₂Cl₂/MeOH/AcOH=100/5/2) to yield the title compound.

### Step B: Synthesis of (R)-2-hydroxy-3,3-dimethyl-4-(naphthalen-2-ylsulfonyl)butanoic acid

To a solution of (R)-2-hydroxy-3,3-dimethyl-4-(naphthalen-2-ylthio)butanoic acid

(1.55 g, 5.35 mmol) in 1,4-dioxane (15 mL) and H₂O (7.5 mL) in ice-water bath was added Oxone monopersulfate (6.58 g, 10.7 mmol) and stirred for 1 h. The reaction mixture was allowed to r.t. and diluted into AcOEt (200 mL) and washed with 1N HCl aq. (200 mL×2) and sat'd NaCl aq. (200 mL×2), dried over Na₂SO₄. The solvent was removed under reduced pressure and dried in vacuo to yield the title compound.

Using the same procedure (2R,3S)-2-hydroxy-3-methyl-4-(3-(trifluoromethyl)-phenylsulfonyl)butanoic acid was prepared from 3-(trifluoromethyl)benzenethiol and (3R,4R)-3-hydroxy-4-methyl-dihydrofuran-2(3H)-one.

### Reference 12

### Synthesis of (2S,3R)-2,3-dihydroxy-4-(3-(trifluoromethyl)phenylsulfonyl)butanoic acid

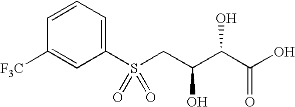

### Step A: Synthesis of (2S,3R)-2,3-dihydroxy-4-(3-(trifluoromethyl)phenylthio)butanoic acid

A mixture of 3-(trifluoromethyl)benzene thiol (0.865 g, 4.9 mmol), (3S,4S)-3,4-dihydroxy-dihydrofuran-2(3H)-one (0.519 g, 4.4 mmol) and potassium carbonate (1.34 g, 9.7 mmol) in DMF (8.0 mL) was stirred under microwave at 120° C. for 30 min. To the reaction mixture was added 1N HCl aq. (100 mL), and extracted with EtOAc (100 mL). The combined organic phase was washed with 1N HCl aq. (100 mL) and sat'd NaCl aq. (100 mL×2), and dried over Na₂SO₄. The solvent was removed under reduced pressure and chromatographed on silica (CH₂Cl₂→CH₂Cl₂/MeOH/AcOH=100/5/1) to yield the title compound.

### Step B: Synthesis of (2S,3R)-2,3-dihydroxy-4-(3-(trifluoromethyl)phenylsulfonyl)butanoic acid

To a solution of (2S,3R)-2,3-dihydroxy-4-(3-(trifluoromethyl)phenylthio)butanoic acid (0.607 g, 2.05 mmol) in 1,4-dioxane (15.0 mL) and H₂O (7.5 mL) in ice-water bath was added Oxone monopersulfate (4.66 g, 7.58 mmol) and the solution was stirred at r.t. The reaction mixture was diluted into AcOEt (150 mL) and washed with 1N HCl aq. (50 mL×2) and sat'd NaCl aq. (50 mL×2), dried over Na₂SO₄. The solvent was removed under reduced pressure and dried in vacuo to yield the title compound.

### Reference 13

### Synthesis of (2R,3S)-2,3-dihydroxy-4-(3-(trifluoromethoxy)phenylsulfonyl)butanoic acid

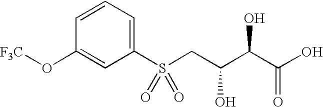

### Step A: Synthesis of (4R,5S)-2,2-dimethyl-5-((3-(trifluoromethoxy)phenylthio)methyl)-1,3-dioxolane-4-carboxylic acid

A mixture of 3-(trifluoromethoxy)benzene thiol (2.064 g, 10.6 mmol), (3aR,6aR)-2,2-dimethyldihydrofuro[3,4-d][1,3]dioxol-4(3aH)-one (1.54 g, 9.73 mmol) and potassium carbonate (2.96 g, 21.4 mmol) in DMF (10.0 mL) was stirred under microwave at 120° C. for 30 min. The reaction mixture was diluted into AcOEt (100 mL) and washed with sat'd NH₄Cl aq./1N HCl aq./sat'd NaCl aq. (200 mL/100 mL/100 mL), sat'd NaCl aq. (100 mL×2), dried over Na₂SO₄. The solvent was removed under reduced pressure and chromatographed on silica (CH₂Cl₂→CH₂Cl₂/MeOH/AcOH=50/5/1) to yield the title compound.

### Step B: Synthesis of (2R,3S)-2,3-dihydroxy-4-(3-(trifluoromethoxy)phenylsulfonyl)-butanoic acid

To a solution of (4R,5S)-2,2-dimethyl-5-((3-(trifluoromethoxy)phenylthio)methyl)-1,3-dioxolane-4-carboxylic acid in 1,4-dioxane (30.0 mL) and H₂O (15.0 mL) was added Oxone monopersulfate (23.44 g, 38.1 mmol) and stirred at r.t. overnight. 4M HCl in 1,4-dioxane (10 mL) was added followed by 1N HCl aq. (45 mL). The reaction mixture was heated to 50° C. for 1.5 h. The reaction mixture was partitioned into AcOEt (300 mL) and 1N HCl aq. (200 mL) and the organic phase was washed with sat'd NaCl aq. (200 mL×2). The organic phase was concentrated under reduced pressure and the residue was re-dissolved in TFA (20 mL), THF (20 mL) and H₂O (20 mL), and stirred at r.t. for 2 h. The solution was diluted with AcOEt (200 mL) and H₂O (200 mL), and the organic phase was washed with sat'd NaCl aq. (100 mL×2), dried over Na₂SO₄. The solvent was removed under reduced pressure and chromatographed on silica (CH₂Cl₂→CH₂Cl₂/MeOH/AcOH=50/5/1) to yield the title compound.

### Reference 14

### Synthesis of (2R,3R)-2,3-dihydroxy-4-(3-(trifluoromethyl)phenylsulfonyl)butanoic acid

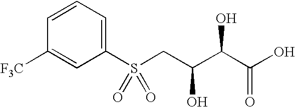

### Step A: Synthesis of (2R,3R)-2,3-dihydroxy-4-(3-(trifluoromethyl)phenylthio)butanoic acid

A mixture of 3-(trifluoromethyl)benzene thiol (0.89 g, 4.97 mmol), (3R,4S)-3,4-dihydroxydihydrofuran-2(3H)-one (0.5364 g, 4.54 mmol) and potassium carbonate (1.517 g, 11.0 mmol) in DMF (8.00 mL) was stirred under microwave at 120° C. for 30 min. The reaction mixture was diluted into AcOEt (80 mL) and washed with 1N HCl aq. (80 mL) and sat'd NaCl aq. (80 mL×2), and dried over Na₂SO₄. The solvent was removed under reduced pressure and chromatographed on silica (CH₂Cl₂→CH₂Cl₂/MeOH/AcOH=50/5/1) to yield the title compound

### Step B: Synthesis of (2R,3R)-2,3-dihydroxy-4-(3-(trifluoromethyl)phenylsulfonyl)-butanoic acid

To a solution of (2R,3R)-2,3-dihydroxy-4-(3-(trifluoromethyl)phenylthio)butanoic acid (0.79 g, 2.66 mmol) in 1,4-dioxane (20.0 mL) and H₂O (10.0 mL) was added Oxone monopersulfate (8.176 g, 13.3 mmol) and stirred overnight. The reaction mixture was diluted into AcOEt (150 mL) and washed with 1N HCl aq. (75 mL) and sat'd NaCl aq. (50 mL×2), dried over Na₂SO₄. The solvent was removed under reduced pressure and the residue was suspended in AcOEt, and hexane was added to form precipitation. This solid was filtered and washed with hexane and dried under vacuum to yield the title compound.

### Reference 15

### Synthesis of (2S,3S)-2,3-dihydroxy-4-(3-(trifluoromethyl)phenylsulfonyl)butanoic acid

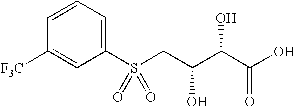

### Step A: Synthesis of (2S,3S)-2,3-dihydroxy-4-(3-(trifluoromethyl)phenylthio)butanoic acid

A mixture of 3-(trifluoromethyl)benzene thiol (0.99 g, 5.53 mmol), (3S,4R)-3,4-dihydroxydihydrofuran-2(3H)-one (0.58 g, 4.92 mmol) and potassium carbonate (1.64 g, 11.9 mmol) in DMF (8.00 mL) was stirred under microwave at 120° C. for 30 min. The reaction mixture was diluted into AcOEt (100 mL) was added and washed with 1N Cl aq. (100 mL) and sat'd NaCl aq. (100 mL×2), and dried over Na₂SO₄. The solvent was removed under reduced pressure and the crude product was chromatographed on silica (CH₂Cl₂—CH₂Cl₂/MeOH/AcOH=50/5/1) to yield the title compound.

### Step B: Synthesis of (2S,3S)-2,3-dihydroxy-4-(3-(trifluoromethyl)phenylsulfonyl)butanoic acid

To a solution of (2S,3S)-2,3-dihydroxy-4-(3-(trifluoromethyl)phenylthio)butanoic acid

(0.66 g, 2.23 mmol) in 1,4-dioxane (20.0 mL) and H₂O (10.0 mL) was added Oxone monopersulfate (6.85 g, 11.1 mmol) and stirred overnight. The reaction mixture was diluted into AcOEt (150 mL) and washed with 1N HCl aq. (75 mL) and sat'd NaCl aq. (50 mL×2), dried over Na₂SO₄. The solvent was removed under reduced pressure and the residue was suspended in AcOEt, and hexane was added to form precipitation. This solid was filtered and washed with hexane and dried under vacuum to yield the title compound.

### Reference 16

### Synthesis of 2,2-dimethyl-4-(3-(trifluoromethyl)phenylsulfonyl)butanoic acid

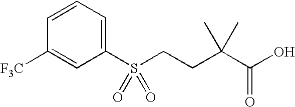

### Step A: Synthesis of 2,2-dimethyl-4-(3-(trifluoromethyl)phenylthio)butanoic acid

A mixture of 3-(trifluoromethyl)benzene thiol (1.64 g, 9.23 mmol), 3,3-dimethyl-dihydrofuran-2(3H)-one (1.00 g, 8.79 mmol) and potassium carbonate (2.88 g, 20.8 mmol) in DMF (5.00 mL) was stirred at 120° C. under microwave in a sealed tube for 1 h. The reaction mixture was partitioned into AcOEt (200 mL) and 1N HCl aq. (200 mL), and the organic layer was washed with 1N HCl aq. (100 mL) and sat'd NaCl aq. (100 mL×2), dried over Na₂SO₄. The solvent was removed under reduced pressure and chromatographed on silica (CH₂Cl₂→CH₂Cl₂/MeOH/AcOH=100/5/1) to yield the title compound.

### Step B: Synthesis of 2,2-dimethyl-4-(3-(trifluoromethyl)phenylsulfonyl)butanoic acid

To a solution of 20 (1.89 g, 6.45 mmol) in 1,4-dioxane (33.0 mL) and H₂O (16.5 mL) was added Oxone monopersulfate (11.9174 g, 19.4 mmol) and stirred overnight. The reaction mixture was partitioned into AcOEt (200 mL) and 1N HCl aq. (200 mL), and the organic layer was washed with 1N HCl aq. (100 mL) and sat'd NaCl aq. (100 mL×2), dried over Na₂SO₄. The solvent was removed under reduced pressure and dried in vacuo to yield the title compound.

### Reference 17

### Synthesis of 4-(naphthalen-2-ylsulfonyl)pentanoic acid

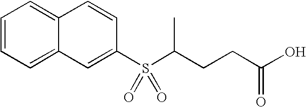

### Step A: Synthesis of ethyl(naphthalen-2-yl)sulfane

Bromoethane (2.8 mL, 37 mmol) and naphthalene-2-thiol (5.00 g, 31 mmol) were dissolved in DMF and potassium carbonate (8.6 g, 62 mmol) was added and the reaction mixture was stirred at room temp overnight. The reaction mixture was quenched with water. (app. 100 mL), and extracted with ethyl acetate (2×100 mL). The extracts were washed with brine (2×25 mL), dried with sodium sulfate, and concentrated to give the title compound (5.8 g, 94%).

### Step B: Synthesis of 2-(ethylsulfonyl)naphthalene.

Ethyl (naphthalen-2-yl)sulfane (5.80 g, 31 mmol) in methanol/water (3:2, 200 mL) was cooled to 0° C. Oxone monopersulfate was added and the reaction mixture was allowed to warm up to 25° C. and stirred for 16 h. The solution was diluted and made acidic with 10% HCl(aq) (200 mL) and then extracted with methylene chloride (3×100 mL), washed with brine (50 mL), dried over sodium sulfate, concentrated and dried in vacuo. The residue was then purified on silica using 0 to 50% EtOAc in hexane to give the title compound (5.7 g, 80% yield).

### Step C: Synthesis of methyl 4-(naphthalen-2-ylsulfonyl)pentanoate

2-(Ethylsulfonyl)naphthalene (1.00 g, 4.54 mmol) is dissolved in THF (25 mL) and cooled to −78 C. n-BuLi (1.2 eq in hexane) was then added and the reaction mixture was allowed to warm to room temp. The reaction mixture was re-cooled to −78 C, and then slowly added to methyl-3-iodopropanoate (1.00 g, 4.54 mmol) in THF (20 mL) at −78 C. The reaction mixture was then allowed to warm to room temp and quenched with sat. sodium bicarbonate. The solvent was removed under vacuum, then the residue was diluted with ethyl acetate, washed with sat. sodium bicarbonate, washed with brine, then dried with sodium sulfate and concentrated. The residue was purified using 0 to 50% ethyl acetate in hexane to give the title compound (190 mg, 13%).

### Step D: Synthesis of 4-(naphthalen-2-ylsulfonyl)pentanoic acid

Methyl 4-(naphthalen-2-ylsulfonyl)pentanoate (190 mg, 0.62 mmol) was dissolved in methanol (2 mL) and then THF (5 mL) and water (5 mL) were added. Lithium hydroxide (29.7 mg, 1.240 mmol) was added and the reaction mixture was stirred at room temp. overnight. 1 M HCl (25 mL) was then added and the product was extracted with ethyl acetate. The extracts were washed with brine, dried with sodium sulfate, and concentrated to give the title compound (160 mg, 88%). MS (ESI, neg. ion) m/z: 290 (M−1).

### Reference 18

### Synthesis of 4-methyl-4-(naphthalen-2-ylsulfonyl)pentanoic acid

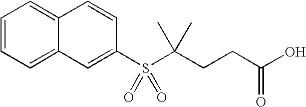

### Step A: Synthesis of isopropyl(naphthalen-2-yl)sulfane

2-Bromopropane (3.5 mL, 37 mmol) and naphthalene-2-thiol (5.00 g, 31 mmol) were dissolved in DMF and potassium carbonate (8.6 g, 62 mmol) was added and the reaction mixture was stirred at room temp. overnight. The reaction mixture was quenched with water (app. 100 mL), and extracted with ethyl acetate (2×100 mL). The extracts were washed with brine (2×25 mL), dried with sodium sulfate, and concentrated to give the title compound.

### Step B: Synthesis of 2-(isopropylsulfonyl)naphthalene

Isopropyl(naphthalen-2-yl)sulfane (6.1 g, 30 mmol) was dissolved in methanol/water (3:2, 200 mL) and was cooled to 0° C. Oxone monopersulfate was added and the reaction mixture was allowed to warm up to 25° C. and stirred for 16 h. The solution was diluted and made acidic with 10% HCl(aq) (200 mL) and extracted with methylenechloride (3×100 mL). The extracts were washed with brine (50 mL), dried over sodium sulfate, concentrated and dried in vacuo. The crude product was then purified on silica using 0 to 50% EtOAc in hexane to give the title compound (4.1 g, 55% yield). The reaction mixture was then azeotroped with toluene.

### Step C: Synthesis of methyl 4-methyl-4-(naphthalen-2-ylsulfonyl)pentanoate

2-(Isopropylsulfonyl)-naphthalene (1.00 g, 4.268 mmol) was dissolved in THF and cooled to −78° C. n-Butyllithium in hexane (2.934 mL, 4.695 mmol) was added and the reaction mixture was warmed to 0 C, then cooled back to −78° C. Methyl 3-iodopropanoate (1.005 g, 4.695 mmol) was dissolved in THF (10 mL), cooled to −78° C., then slowly added to the sulfone anion. The reaction mixture is then stirred and allowed to warm to room temp. and quenched with sat. sodium bicarbonate. The THF was removed under vacuum, then the reaction mixture was diluted with ethyl acetate, washed with sat. sodium bicarbonate, washed with brine, then dried with sodium sulfate and concentrated. The reaction mixture was purified using 0 to 50% ethyl acetate in hexane to give the title compound.

### Step D: Synthesis of 4-methyl-4-(naphthalen-2-ylsulfonyl)pentanoic acid

Methyl 4-methyl-4-(naphthalen-2-ylsulfonyl)pentanoate (270 mg, 0.843 mmol) was dissolved in methanol (2 mL), then THF (5 mL) and water (5 mL) were added. Lithium hydroxide (40 mg, 1.685 mmol) was added and the reaction mixture was stirred at room temp. overnight. 1 M HCl (25 mL) was then added and the product was extracted with ethyl acetate, then the extracts were washed with brine, dried with sodium sulfate, and concentrated to give the title compound.

### Reference 19

### Synthesis of 3-hydroxy-4-methyl-4-(3-(trifluoromethyl)phenylsulfonyl)pentanoic acid

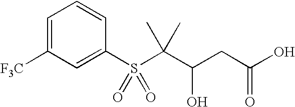

### Step A: Synthesis of methyl 2-(3-(trifluoromethyl)phenylthio)acetate

To a suspension of potassium carbonate (1.02 mL, 16.8 mmol) in DCM, was added 3-(trifluoromethyl)benzene thiol (2.00 g, 11.2 mmol) and methyl 2-bromoacetate (1.17 mL, 12.3 mmol) and the reaction mixture was stirred for two h. Water (25 mL) was added and the reaction mixture was extracted with DCM. The extracts were washed with brine, then dried with sodium sulfate and concentrated to give the title compound.

### Step B: Synthesis of tert-butyl 3-hydroxy-4-methyl-4-(3-(trifluoromethyl)phenylsulfonyl)-pentanoate

To a solution of LDA (3.6 mmol) in dry THF (10 mL) at −78° C. was added tert-butyl acetate (332 mg, 2.854 mmol) and the solution was stirred at 0° C. for 1 h. To this solution at −78° C. was added 2-methyl-2-(3-(trifluoromethyl)phenylsulfonyl)propanal (200 mg, 0.714 mmol) in dry THF (5 mL) and the reaction was stirred at −78° C. for 15 min. The reaction was quenched with sat. NH₄Cl and extracted with EtOAc, dried over Na₂SO₄ and evaporated. The crude product was purified on silica using 5 to 40% EtoAc in hexane to give the title compound.

### Step C: Synthesis of 3-hydroxy-4-methyl-4-(3-(trifluoromethyl)phenylsulfonyl)-pentanoic acid

Tert-butyl 3-hydroxy-4-methyl-4-(3-(trifluoromethyl)phenylsulfonyl)pentanoate (165 mg, 0.416 mmol) was dissolved in 1M HCl in diethyl ether and the reaction mixture was stirred over the weekend. The reaction mixture was concentrated and the residue was purified on silica using 30 to 100% EtOAc in hexane to give the title compound.

### Example 1

### Synthesis of (S,R)-4-(naphthalen-2-ylsulfonyl)-3-phenyl-N-((R)-5-(piperidin-1-ylmethyl)-2,3-dihydro-1H-inden-1-yl)butanamide

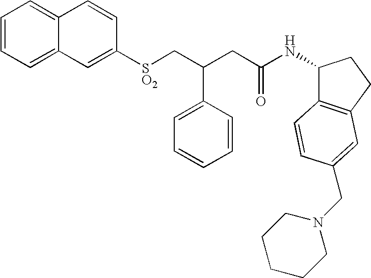

### Step A: Synthesis of 4-(naphthalen-2-ylthio)-3-phenylbutanoic acid

A solution of 4-phenyldihydrofuran-2(3H)-one (5 g, 30.85 mmol) and naphthalene-2-thiol (7.4 g, 46.2 mmol, 1.5 equiv) in N,N-dimethylformamide (100 mL) was cooled to 0° C. and treated with sodium hydride (2.1 g of a 60% dispersion in mineral oil, 52.4 mmol, 1.7 equiv). The reaction mixture was allowed to warm to room temperature over 1 h, and then heated to 100° C. for 18 h. After cooling to room temperature, the reaction mixture was diluted with ethyl acetate (200 mL), washed with 10% hydrochloric acid solution (100 mL), water (100 mL), brine (100 mL), dried (MgSO₄) and purified on silica gel using 25-50% ethyl acetate in hexane as eluant, affording the title compound. MS: 323.1 (M+H)⁺.

### Step B: Synthesis of 4-(naphthalen-2-ylsulfonyl)-3-phenylbutanoic acid.

A solution of 4-(naphthalen-2-ylthio)-3-phenylbutanoic acid (7.9 g, 24.5 mmol) in methanol (180 mL) and dioxane (30 mL) was treated with a solution of Oxone (44.0 g, 73.0 mmol, 3.0 equiv) in water (220 mL). After being stirred at room temperature for 4 h, the reaction mixture was diluted with ethyl acetate (400 mL) and washed with 10% hydrochloric acid solution (250 mL), water (250 mL), brine (250 mL), dried (MgSO₄) and concentrated, affording the title compound.

### Step C: Synthesis of N-((R)-5-(hydroxymethyl)-2,3-dihydro-1H-inden-1-yl)-4-(naphthalen-2-ylsulfonyl)-3-phenylbutanamide

A solution of 4-(naphthalen-2-ylsulfonyl)-3-phenylbutanoic acid (500 mg, 1.41 mmol) in dichloromethane (15 mL) was treated with DMF (0.01 mL, 0.14 mmol, 0.1 equiv) and oxalyl chloride (0.77 mL of a 2.0M solution in dichloromethane, 1.55 mmol, 1.1 equiv). After stirring at room temperature for 30 min, the reaction mixure was concentrated in vacuo. The crude acid chloride was dissolved in tetrahydrofuran (4 mL) and was transferred to a solution of (R)-(1-amino-2,3-dihydro-1H-inden-5-yl)methanol (254 mg, 1.55 mmol, 1.1 equiv) and sodium carbonate (1.5 g, 14.1 mmol, 10.0 equiv) in tetrahydrofuran (4 mL) and water (8 mL). After being stirred at room temperature for 14 h, the reaction mixure was diluted with ethyl acetate (20 mL) and washed with 10% hydrochloric acid solution (3×20 mL), sodium hydroxide solution (1.0N, 20 mL), brine (20 mL), dried (MgSO₄) and concentrated affording the title compound.

### Step D: Synthesis of N-((R)-5-formyl-2,3-dihydro-1H-inden-1-yl)-4-(naphthalen-2-yl-sulfonyl)-3-phenylbutanamide

N-((R)-5-(hydroxymethyl)-2,3-dihydro-1H-inden-1-yl)-4-(naphthalen-2-ylsulfonyl)-3-phenylbutanamide (680 mg, 1.36 mmol) was dissolved in dichloromethane (40 mL) and N,N-dimethylformamide (8 mL) and was treated with manganese (IV) oxide (3.0 g). After stirring at room temperature for 2 h, the reaction mixure was filtered through celite, concentrated and purified on silica gel using 40-65% ethyl acetatel in hexane as eluant, affording the title compound.

### Step E: Synthesis of (S,R)-4-(naphthalen-2-ylsulfonyl)-3-phenyl-N-((R)-5-(piperidin-1-ylmethyl)-2,3-dihydro-1H-inden-1-yl)butanamide

A solution of N-((R)-5-formyl-2,3-dihydro-1H-inden-1-yl)-4-(naphthalen-2-ylsulfonyl)-3-phenylbutanamide (498 mg, 1.0 mmol) and piperidine (2 mL, 20 mmol, 20 equiv) in chloroform (20 mL) was heated to 50° C. and treated with sodium triacetoxyborohydride (2.1 g, 10 mmol, 10.0 equiv). The reaction mixure was cooled to room temperature after 150 min, diluted with dichloromethane (20 mL), washed with saturated sodium bicarbonate solution (2×20 mL), brine (20 mL), dried (MgSO₄) and purified on silica gel using 10% methanol in dichloromethane as eluant, affording the title compound. MS: 567.3 (M+H)⁺.

### Example 2

### Synthesis of (S)-3-hydroxy-4-(naphthalen-2-ylsulfonyl)-N-((R)-6-(piperidin 1-ylmethyl)-1,2,3,4-tetrahydronaphthalen-1-yl)butanamide

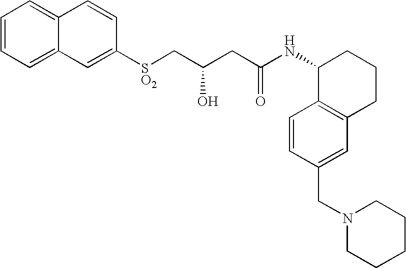

### Step A: Synthesis of (S)-methyl 3-hydroxy-4-(naphthalen-2-ylthio)butanoate

A solution of (S)-ethyl 2-(oxiran-2-yl)acetate (457 mg, 3.51 mmol) and naphthalene-2-thiol (731 mg, 4.56 mmol, 1.3 equiv) in methanol (25 mL) was treated with sodium carbonate (930 mg, 8.77 mmol, 2.5 equiv). The reaction mixure was stirred at room temperature for 20 h, diluted with ethyl acetate (30 mL), washed with saturated ammonium chloride solution (30 mL), brine (30 mL), dried (MgSO₄) and purified on silica gel using 10-25% ethyl acetate in hexane as eluant, affording (S)-methyl 3-hydroxy-4-(naphthalen-2-ylthio)butanoate.

### Step B: Synthesis of (S)-methyl 3-hydroxy-4-(naphthalen-2-ylsulfonyl)butanoate

A solution of (S)-methyl 3-hydroxy-4-(naphthalen-2-ylthio)butanoate (650 mg, 2.35 mmol) in methanol (15 mL) at 0° C. was treated with a solution of Oxone (3.6 g, 5.88 mmol, 2.5 equiv) in water (10 mL). The reaction mixure was allowed to warm to room temperature over 5 h, diluted with ethyl acetate (40 mL) and washed with saturated sodium bicarbonate solution (2×25 mL), brine (25 mL), dried (MgSO₄) and concentrated, affording the title compound.

### Step C: Synthesis of (S)-3-hydroxy-4-(naphthalen-2-ylsulfonyl)butanoic acid

A solution of (S)-methyl 3-hydroxy-4-(naphthalen-2-ylsulfonyl)butanoate (237 mg, 0.77 mmol) in tetrahydrofuran (5 mL) and water (2 mL) was treated with lithium hydroxide monohydrate (49 mg, 1.15 mmol, 1.5 equiv). After stirring at room temperature for 15 h, the reaction mixure was quenched by Dowex-50 acidic ion-exchange resin, filtered and concentrated in vacuo. The crude acid was dried by azeotroping with toluene (2×5 mL), affording the title compound.

### Step D: Synthesis of (S)-3-yydroxy-4-(naphthalen-2-ylsulfonyl)-N-((R)-6-(piperidin-1-ylmethyl)-1,2,3,4-tetrahydronaphthalen-1-yl)butanamide

A solution of (S)-3-hydroxy-4-(naphthalen-2-ylsulfonyl)butanoic acid (190 mg, 0.64 mmol) and (R)-6-(piperidin-1-ylmethyl)-1,2,3,4-tetrahydronaphthalen-1-amine dihydrochloride salt (224 mg, 0.71 mmol, 1.1 equiv) in DMF (6 mL) was treated with coupling agent HATU (365 mg, 0.96 mmol, 1.5 equiv) and N,N-diisopropylethylamine (0.56 mL, 3.2 mmol, 5.0 equiv). The reaction mixure was stirred at room temperature for 18 h, after which another aliquot of HATU was added (243 mg, 0.64 mmol, 1.0 equiv). After a further 5 h, the reaction mixure was diluted with ethyl acetate (15 ml), washed with saturated sodium bicarbonate solution (3×10 mL), brine (15 mL), dried (MgSO₄) and purified on silica gel using 4-8% methanol in dichloromethane as eluant, affording the title compound. MS: 521.3 (M+H)⁺.

### Example 3

### Synthesis of (S)-3-amino-N-((R)-7-((tert-butylamino)methyl)-6-chlorochroman-4-yl)-4-(3,4-dichlorophenylsulfonyl)butanamide

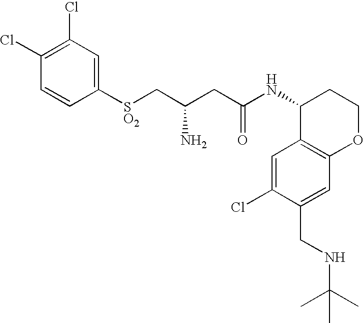

### Step A: Synthesis of (S)-benzyl 3-(tert-butoxycarbonylamino)-4-(3,4-dichlorophenylthio)butanoate

A solution of (S)-benzyl 3-(tert-butoxycarbonylamino)-4-hydroxybutanoate (500 mg, 1.6 mmol) in dichloromethane (15 mL) was cooled to 0° C. and treated with triethylamine (0.34 mL, 2.41 mmol, 1.5 equiv) and methanesulfonyl chloride (0.14 mL, 1.77 mmol, 1.1 equiv). After 2 h, the reaction mixure was warmed to room temperature, diluted with ethyl acetate (25 ml), washed with saturated ammonium chloride solution (15 mL), brine (15 mL), dried (MgSO₄) and concentrated in vacuo. 3,4-dichlorobenzenethiol (0.31 mL, 2.4 mmol, 1.5 equiv) in N,N-dimethylformamide (5 mL) was cooled to 0° C. and treated with sodium hydride (105 mg of a 60% dispersion in mineral oil, 2.56 mmol, 1.6 equiv). After 5 min, a solution of the crude mesylate in N,N-dimethylformamide (5 mL) was added to the reaction mixture. The reaction mixure was warmed to room temperature and stirred for 24 h. The suspension was diluted with ethyl acetate (25 ml), washed with saturated ammonium chloride solution (15 mL), water (15 mL), brine (15 mL), dried (MgSO₄) and purified on silica gel using 5-20% ethyl acetate in hexane as eluant, affording the title compound.

### Step B: Synthesis of (S)-benzyl 3-(tert-butoxycarbonylamino)-4-(3,4-dichlorophenylsulfonyl)butanoate

A solution of (S)-benzyl 3-(tert-butoxycarbonylamino)-4-(3,4-dichlorophenylthio)-butanoate (552 mg, 1.17 mmol) in methanol (20 mL) and water (10 mL) was cooled to 0° C. and treated with sodium bicarbonate (980 mg, 11.7 mmol, 10.0 equiv) and Oxone (1.44 g, 2.35 mmol, 2.0 equv). After 3 h, the reaction mixure was diluted with saturated sodium bicarbonate solution (25 mL) and extracted with ethyl acetate (3×25 mL). The combined organic layers were washed with brine (20 mL), dried (MgSO₄) and concentrated in vacuo to afford (S)-benzyl 3-(tert-butoxycarbonyl)-4-(3,4-dichlorophenylsulfonyl)butanoate. MS: 502.1 (M+H)⁺.

### Step C: Synthesis of (S)-3-(tert-Butoxycarbonyl)-4-(3,4-dichlorophenylsulfonyl)-butanoic acid

A solution of (S)-benzyl 3-(tert-butoxycarbonyl)-4-(3,4-dichlorophenylsulfonyl)-butanoate (560 mg, 1.1 mmol) in THF (10 mL) and water (3 mL) was treated with lithium hydroxide monohydrate (95 mg, 2.2 mmol, 2.0 equiv). The reaction mixure was stirred at room temperature for 6 h, diluted with 1% hydrochloric acid (10 mL) and extracted with dichloromethane (3×15 mL). The combined organic layers were dried (MgSO₄) and concentrated in vacuo, affording the title compound.

### Step D: Synthesis of tert-butyl (S)-4-((R)-7-((tert-butylamino)methyl)-6-chlorochroman-4-ylamino)-1-(3,4-dichlorophenylsulfonyl)-4-oxobutan-2-ylcarbamate

A solution of (S)-3-(tert-butoxycarbonylamino)-4-(3,4-dichlorophenylsulfonyl)butanoic acid (435 mg, 1.05 mmol) and (R)-7-((tert-butylamino)methyl)-6-chlorochroman-4-amine (423 mg, 1.57 mmol, 1.49 equiv) in DMF (15 mL) was treated with the peptide coupling agent HATU (605 mg, 1.58 mmol, 1.5 equiv) and N,N-diisopropylethylamine (0.55 mL, 3.15 mmol, 3.0 equiv). After stirring at room temperature for 2 h, the reaction mixure was diluted with ethyl acetate (25 mL), washed with 5% citric acid solution (15 mL), saturated sodium bicarbonate solution (15 mL), brine (15 mL), dried (MgSO₄) and purified on silica gel using 4-8% methanol in dichloromethane as eluant, affording the title compound.

### Step E: Synthesis of (S)-3-amino-N-((R)-7-((tert-butylamino)methyl)-6-chlorochroman-4-yl)-4-(3,4-dichlorophenylsulfonyl)butanamide

A solution of tert-butyl (S)-4-((R)-7-((tert-butylamino)methyl)-6-chlorochroman-4-ylamino)-1-(3,4-dichlorophenylsulfonyl)-4-oxobutan-2-ylcarbamate (340 mg, 0.51 mmol) in methanol (10 mL) and dichloromethane (2 mL) was treated with hydrogen chloride (2.5 mL of a 11.0M solution in diethyl ether, 2.5 mmol, 5.0 equiv). After 20 h, the reaction mixure was concentrated in vacuo, affording the title compound. MS: 562.3 (M+H)⁺.

### Example 4

### Synthesis of (S)-3-methyl-4-(naphthalen-2-ylsulfonyl)-N-((R)-6-(piperidin-1-ylmethyl)-1,2,3,4-tetrahydronaphthalen-1-yl)butanamide

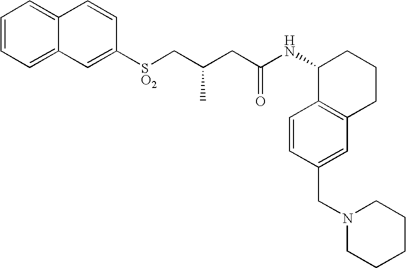

### Step A: Synthesis of (S)-4-(tert-butyldiphenylsilyloxy)-2-methylbutan-1-ol

A solution of (S)-2-methylbutane-1,4-diol (2.5 g, 24.01 mmol) in DMF (100 mL) was treated with tert-butyldiphenylsilyl chloride (6.25 mL, 24.01 mmol, 1.0 equiv) and cooled to −50° C. 1,8-Diazabicyclo[5.4.0]undec-7-ene (5.1 mL, 36.01 mmol, 1.5 equiv) was added in a dropwise fashion. After 20 min, the reaction mixure was diluted with ethyl acetate (500 mL), warmed to room temperature, washed with saturated ammonium chloride solution (100 mL), saturated sodium bicarbonate solution (100 mL), brine (100 mL), dried (MgSO₄) and purified on silica gel using 5-10% ethyl acetate in hexane as eluant, affording the title compound.

### Step B: Synthesis of (S)-tert-butyl (3-methyl-4-(naphthalen-2-ylthio)butoxy)-diphenylsilane

A solution of (S)-4-(tert-butyldiphenylsilyloxy)-2-methylbutan-1-ol (911 mg, 2.66 mmol) in dichloromethane (20 mL) was cooled to 0° C. and treated with triethylamine (0.75 mL, 5.32 mmol, 2.0 equiv) and methanesulfonyl chloride (0.25 mL, 3.19 mmol). The reaction mixture was warmed to room temperature after 2 h, diluted with brine (25 mL) and extracted with ethyl acetate (2×25 mL). The combined organic layers were dried (MgSO₄) and concentrated in vacuo. The crude mesylate was dissolved in DMF (20 mL), treated with naphthalene-2-thiol (541 mg, 3.38 mmol) and cooled to 0° C. Sodium hydride (160 mg of a 60% dispersion in mineral oil, 3.9 mmol) was added, and the reaction mixure was allowed to warm to room temperature over 15 min. After 20 h, the reaction mixure was diluted with ethyl acetate (40 mL), washed with 10% hydrochloric acid solution (25 mL), saturated sodium bicarbonate solution (25 mL), brine (25 mL), dried (MgSO₄) and purified on silica gel using 2% ethyl acetate in hexane as eluant, affording the title compound.

### Step C: Synthesis of (S)-3-methyl-4-(naphthalen-2-ylthio)butan-1-ol

A solution of (S)-tert-butyl (3-methyl-4-(naphthalen-2-ylthio)butoxy)diphenylsilane (1.08 g, 2.23 mmol) in THF (20 mL) was treated with tetrabuytlammonium fluoride (3.4 mL of a 1.0 M solution in tetrahydrofuran, 3.4 mmol). The reaction mixure was stirred at room temperature for 17 h, diluted with ethyl acetate (30 mL), washed with 10% hydrochloric acid solution (25 mL), saturated sodium bicarbonate solution (25 mL), brine (25 mL), dried (MgSO₄) and purified on silica gel using 20-35% ethyl acetate in hexane as eluant, affording the title compound. MS: 247.2 (M+H)⁺.

### Step D: Synthesis of (S)-3-methyl-4-(naphthalen-2-ylsulfonyl)butan-1-ol

A solution of (S)-3-methyl-4-(naphthalen-2-ylthio)butan-1-ol (450 mg, 1.82 mmol) in methanol (20 mL) and water (15 mL) was cooled to 0° C. and treated with Oxone (2.8 g, 4.57 mmol, 2.5 equiv). The reaction mixure was allowed to warm to room temperature over 3 h, diluted with ethyl acetate (40 mL), washed with saturated sodium bicarbonate solution (2×25 mL), brine (25 mL), dried (MgSO₄) and concentrated in vacuo affording the title compound. MS: 279.2 (M+H)—⁺.

### Step E: Synthesis of (S)-3-methyl-4-(naphthalen-2-ylsulfonyl)butanoic acid

A solution of (S)-3-methyl-4-(naphthalen-2-ylsulfonyl)butan-1-ol (500 mg, 1.8 mmol) in acetone (20 mL) was treated with Jones reagent (also known as chromic acid, prepared from CrO₃ and sulfuric acid as described in Encyclopedia of Reagents for Organic Synthesis, Leo Paquette Ed., Wiley, 1995) till the orange color of the reagent persisted and did not change to green (1.1 mL added). The reaction mixture was diluted with ethyl acetate (30 mL), decanted into a separatory funnel, washed with water (2×25 mL), brine (25 mL), dried (MgSO₄) and concentrated in vacuo affording the title compound. MS: 293.2 (M+H)⁺.

### Step F: Synthesis of (S)-3-methyl-4-(naphthalen-2-ylsulfonyl)-N-((R)-6-(piperidin-1-ylmethyl)-1,2,3,4-tetrahydronaphthalen-1-yl)butanamide

A solution of (S)-3-methyl-4-(naphthalen-2-ylsulfonyl)butanoic acid (470 mg, 1.6 mmol) and (R)-6-(piperidin-1-ylmethyl)-1,2,3,4-tetrahydronaphthalen-1-amine (400 mg, 1.6 mmol, 1.0 equiv) in dichloromethane (15 mL) was treated with peptide coupling agents HOBT (332 mg, 2.4 mmol, 1.5 equiv) and EDCI (471 mg, 2.4 mmol, 1. equiv). The reaction mixure was stirred at room temperature for 15 h, diluted with ethyl acetate (30 mL), washed with saturated sodium bicarbonate solution (25 mL), brine (25 mL), dried (MgSO₄) and purified on silica gel using 5% methanol in dichloromethane as eluant, affording the title compound. MS: 519.4 (M+H)⁺.

Similarly, the compounds in Table 1 may be prepared.

TABLE 1

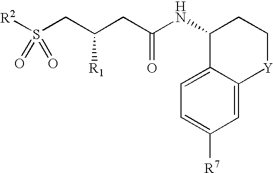

| R² | R¹ | Y | R⁷ |
|---|---|---|---|
| 3-CF₃-phenyl | CH₃ | CH₂ | piperidin-1-ylmethyl |
| 3-CF₃-phenyl | CH₃ | CH₂ | N-Mepiperazin-1-ylmethyl |
| 3-CF₃-phenyl | CH₃ | CH₂ | 4-Mepiperidin-1-ylmethyl |
| 3-CF₃-phenyl | CH₃ | CH₂ | 3-Mepiperidin-1-ylmethyl |
| 3-CF₃-phenyl | CH₃ | CH₂ | azepan-1-ylmethyl |
| 3-CF₃-phenyl | CH₃ | CH₂ | 3-hydroxy- |
| | | | piperidin-1-ylmethyl |
| 3-CF₃-phenyl | CH₃ | CH₂ | 3-HOCH₂piperidin-1-ylmethyl |
| 3-Cl-phenyl | CH₃ | CH₂ | piperidin-1-ylmethyl |
| 2-F-5-CF₃-phenyl | CH₃ | CH₂ | piperidin-1-ylmethyl |
| 2-Cl-5-CF₃-phenyl | CH₃ | CH₂ | piperidin-1-ylmethyl |
| 2-Naphthyl-phenyl | CH₃ | CH₂ | piperidin-1-ylmethyl |
| 2,3-diCl-phenyl | CH₃ | CH₂ | piperidin-1-ylmethyl |
| 2,5-diCl-phenyl | CH₃ | CH₂ | piperidin-1-ylmethyl |
| 3,4-diCl-phenyl | CH₃ | CH₂ | piperidin-1-ylmethyl |
| 2,6-diCl-phenyl | CH₃ | CH₂ | piperidin-1-ylmethyl |
| 2-F-5-Cl-phenyl | CH₃ | CH₂ | piperidin-1-ylmethyl |
| 2-F-3-Cl-phenyl | CH₃ | CH₂ | piperidin-1-ylmethyl |
| 2,5-diMe-4-Cl-phenyl | CH₃ | CH₂ | piperidin-1-ylmethyl |
| 4-tert-Bu-phenyl | CH₃ | CH₂ | piperidin-1-ylmethyl |
| 2,5-diMe-4-Cl-phenyl | CH₃ | O | piperidin-1-ylmethyl |
| 2,5-diMe-4-Cl-phenyl | CH₃ | — | piperidin-1-ylmethyl |
| 2,5-diMe-4-Cl-phenyl | CF₃ | — | piperidin-1-ylmethyl |
| 4-Cl-phenyl | CF₃ | CH₂ | piperidin-1-ylmethyl |
| 2-Cl-phenyl | CF₃ | CH₂ | piperidin-1-ylmethyl |
| 2-OCF₃-phenyl | CF₃ | CH₂ | piperidin-1-ylmethyl |
| 3-OMe-phenyl | CF₃ | CH₂ | piperidin-1-ylmethyl |
| 3-OCF₃-phenyl | CF₃ | CH₂ | piperidin-1-ylmethyl |
| 2,3-diCl-phenyl- | CF₃ | CH₂ | azepan-1-ylmethyl |
| phenyl | | | |
| 3-CF₃-phenyl | CF₃ | CH₂ | azepan-1-ylmethyl |
| 3-CF₃-phenyl | CF₃ | CH₂ | piperidin-1-ylmethyl |
| 3-CF₃-phenyl | CF₃ | CH₂ | 4-Mepiperidin-1-ylmethyl |
| 3-CF₃-phenyl | CF₃ | CH₂ | 3-OHpiperidin-1-ylmethyl |
| 3-CF₃-phenyl | CF₃ | CH₂ | 3-HOCH₂piperidin-1-ylmethyl |
| phenyl | CF₃ | CH₂ | piperidin-1-ylmethyl |
| 2,5-diCl-phenyl | CF₃ | CH₂ | piperidin-1-ylmethyl |
| 3-Me-phenyl | CF₃ | CH₂ | piperidin-1-ylmethyl |
| 3-Cl-phenyl | CF₃ | CH₂ | piperidin-1-ylmethyl |
| 2-F-4-Cl-phenyl | CF₃ | CH₂ | piperidin-1-ylmethyl |
| 2-F-3-Cl-phenyl | CF₃ | CH₂ | piperidin-1-ylmethyl |
| 2-F-5-Cl-phenyl | CF₃ | CH₂ | piperidin-1-ylmethyl |
| 2,3-diCl-phenyl | CF₃ | CH₂ | piperidin-1-ylmethyl |
| 2,6-diCl-phenyl | CF₃ | CH₂ | piperidin-1-ylmethyl |
| 3,4-diCl-phenyl | CF₃ | CH₂ | piperidin-1-ylmethyl |
| 3,5-diCl-phenyl | CF₃ | CH₂ | piperidin-1-ylmethyl |
| 2-Cl-3-CF₃-phenyl | CF₃ | CH₂ | piperidin-1-ylmethyl |
| 2-naphthylphenyl | CF₃ | CH₂ | piperidin-1-ylmethyl |
| 2-naphthylphenyl | CF₃ | O | piperidin-1-ylmethyl |
| 2,5-diMe-4-Cl-phenyl | CF₃ | O | piperidin-1-ylmethyl |
| 2-naphthylphenyl | Et | CH₂ | piperidin-1-ylmethyl |
| 2-naphthylphenyl | i-Pr | CH₂ | piperidin-1-ylmethyl |
| 2-naphthylphenyl | c-Pr | CH₂ | piperidin-1-ylmethyl |
| 2-naphthylphenyl | tert-Bu | CH₂ | piperidin-1-ylmethyl |
| 2-naphthylphenyl | CN | CH₂ | piperidin-1-ylmethyl |
| 2-naphthylphenyl | CCH | CH₂ | piperidin-1-ylmethyl |
| 2-naphthylphenyl | CH₂CCH | CH₂ | piperidin-1-ylmethyl |
| 2-naphthylphenyl | CH₂F | CH₂ | piperidin-1-ylmethyl |
| 2-naphthyl-phenyl | CH₂CN | CH₂ | piperidin-1-ylmethyl |

### Example 5

### Synthesis of (R)-3-(hydroxymethyl)-4-(naphthalen-2-ylsulfonyl)-N-(6-(piperidin-1-ylmethyl)-1,2,3,4-tetrahydronaphthalen-1-yl)butanamide

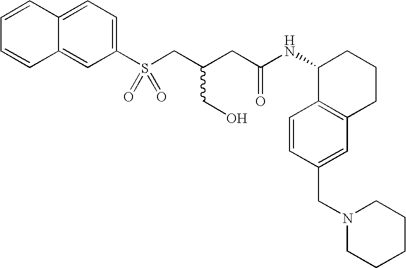

### Step A: Synthesis of 4-methoxy-2-((naphthalen-2-ylsulfonyl)methyl)-4-oxobutanoic acid

Itaconic acid methyl ester (2 g, 13.8 mmol), 2-naphthalenethiol (2.4 g, 15.3 mmol), piperidine (0.41 mL, 4.14 mmol) in dioxane (20 mL) were heated to reflux for 8 h. The reaction mixture was diluted with ethyl acetate and washed with 110% HCl, brine, and dried and concentrated to give the oil. Column chromatograph purification gave the title compound.

### Step B: Synthesis of methyl 3-(hydroxymethyl)-4-(naphthalen-2-ylthio)butanoate and 4-((naphthalen-2-ylthio)methyl)-dihydrofuran-2(3H)-one

To a solution of 4-methoxy-2-((naphthalen-2-ylsulfonyl)methyl)-4-oxobutanoic acid (2.94 g, 9.66 mmol) in THF (50 mL) under N₂ at −10° C. was added isobutyl carbonochloridate (1.30 mL, 10.0 mmol) followed by 4-methylmorpholine (1.10 mL, 10.0 mmol). After 10 min, sodium borohydride (1.10 g, 29.1 mmol) was slowly added. After 20 min, the reaction mixture was quenched with sat'd NH₄Cl aq. (400 mL) and extracted with AcOEt (400 mL). The organic layer was washed with sat'd NaCl aq. (400 mL×2) and dried over Na₂SO₄. The solvent was removed under reduced pressure and chromatographed on silica (hexane→hexane/AcOEt=2/1) to yield a mixture of above compounds.

### Step C: Synthesis of 2-((naphthalen-2-ylthio)methyl)succinic acid and 4-((naphthalen-2-yl-thio)methyl)-dihydrofuran-2(3H)-one

A mixture of methyl 3-(hydroxymethyl)-4-(naphthalen-2-ylthio)butanoate and 4-((naphthalen-2-ylthio)methyl)-dihydrofuran-2(3H)-one (3.24 g) was dissolved in THF (40 mL) and diluted with MeOH (40 mL) and H₂O (40 mL). Lithium hydroxide monohydrate (3.85 g, 91.7 mmol) was added and stirred at r.t. for 1 h. The solvent was removed under reduced pressure and the residue was stirred in AcOEt (100 mL) and H₂O (50 mL) vigorously, then acidified with 5 N HCl aq. to pH 1. The organic phase was collected and washed with a mixture of sat'd NaCl aq. and 1N HCl aq. (50 mL+50 mL×2) and dried over Na₂SO₄. The solvent was removed under reduced pressure and dried in vacuo to yield a mixture of the title compounds.

### Step D: Synthesis of 4-((naphthalen-2-ylthio)methyl)-dihydrofuran-2(3H)-one

A mixture of 2-((naphthalen-2-ylthio)methyl)succinic acid and 4-((naphthalen-2-yl-thio)methyl)-dihydrofuran-2(3H)-one (3.79 g) was stirred in 1M HCl solution in Et₂O for 2 h, and then quenched with Na₂SO₄ (30 g) and stirred vigorously. The reaction mixture was filtered and the filtrate was condensed under reduced pressure. The residue was suspended in AcOEt (200 mL) and washed with sat'd NaCl aq. (200 mL×2) and dried over Na₂SO₄. The solvent was removed under reduced pressure and chromatographed on silica (CH₂Cl₂) to yield the title compound.

### Step E: Synthesis of 4-((naphthalen-2-ylsulfonyl)methyl)-dihydrofuran-2(3H)-one

A mixture of 4-((naphthalen-2-ylthio)methyl)-dihydrofuran-2(3H)-one (0.474 g, 1.84 mmol) and Oxone (2.25 mmol) in 1,4-dioxane (5.0 mL) and H₂O (2.5 mL) was stirred at r.t. for 20 h. The reaction mixture was quenched with H₂O (50 mL) and sat'd NaCl aq. (50 mL) and extracted with AcOEt (100 mL). The organic layer was washed with sat'd NaCl aq. (100 mL×2) and dried over Na₂SO₄. The solvent was removed under reduced pressure and the residual solid was recrystallized from AtOEc/CH₂Cl₂/hexane and dried under vacuum to yield the title compound.

### Step F: Synthesis of (R)-3-(hydroxymethyl)-4-(naphthalen-2-ylsulfonyl)-N-(6-(piperidin-1-ylmethyl)-1,2,3,4-tetrahydronaphthalen-1-yl)butanamide

To a solution of (R)-6-(piperidin-1-ylmethyl)-1,2,3,4-tetrahydronaphthalen-1-amine (0.124 g, 0.507 mmol) in CH₂Cl₂ at 0° C. was added trimethylaluminum (2M solution in toluene, 0.510 mL, 1.02 mmol) followed by 4-((naphthalen-2-ylsulfonyl)methyl)-dihydrofuran-2(3H)-one (0.157 g, 0.540 mmol). After 15 min, the cold bath was removed and the solution was heated to 50° C. for 17 h. 4-((Naphthalen-2-ylsulfonyl)methyl)-dihydrofuran-2(3H)-one (0.155 g, 0.533 mmol) and trimethylaluminum (2M solution in toluene, 0.510 mL, 1.02 mmol) were added and the reaction was further stirred at 70° C. for 1.5 h. The reaction mixture was allowed to r.t., and quenched with AcOEt (20 mL) and sat'd NH₄Cl aq. (20 mL). The reaction mixture was diluted with AcOEt (80 mL), sat'd NH₄Cl aq. (40 mL) and sat'd NaHCO₃ aq. (40 mL), and filtered to remove the insoluble materials. The organic layer of the filtrate was washed with sat'd NaHCO₃ aq. (100 mL) and sat'd NaCl aq. (100 mL×2), and dried over Na₂SO₄. The solvent was removed under reduced pressure and chromatographed on silica (CH₂Cl₂→CH₂Cl₂/MeOH with 2N NH₃=40/1) to yield the title compound.

### Example 6

### Synthesis of (2S,3R)-2,3-dihydroxy-N-((R)-6-(1-methylpiperidin-1-ylmethyl)-1,2,3,4-tetrahydronaphthalen-1-yl)-4-(3-(trifluoromethyl)phenylsulfonyl)butanamide iodide

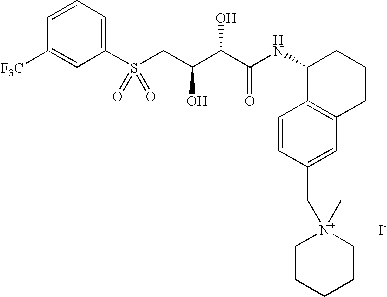

To a solution of (2S,3R)-2,3-dihydroxy-N-((R)-6-(piperidin-1-ylmethyl)-1,2,3,4-tetrahydronaphthalen-1-yl)-4-(3-(trifluoromethyl)phenylsulfonyl)butanamide (0.0844 g, 0.152 mmol) in dichloromethane (4.00 mL) was added iodomethane (0.100 mL, 1.60 mmol) and stirred at 50° C. in a sealed tube for 2 h. After cooling to r.t., the solution was concentrated under reduced pressure and the residual solid was treated with Et₂O, sonicated, filtered and washed with Et₂O and dried under vacuum to yield the title compound.

### Example 7

### Synthesis of (2R,3S)-2,3-dihydroxy-4-(naphthalen-2-ylsulfonyl)-N-((R)-6-(piperidin-1-yl-methyl)-1,2,3,4-tetrahydronaphthalen-1-yl)butanamide

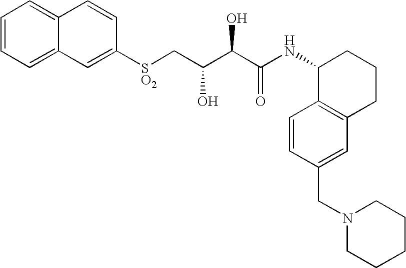

### Step A: Synthesis of (4R,5S)-2,2-dimethyl-5-((naphthalen-2-ylthio)methyl)-1,3-dioxolane-4-carboxylic acid

A solution of 2-naphthalenethiol (3.5 g, 22 mmol) in DMF (60 mL) was cooled to 0° C. and treated with sodium hydride, 60% dispersion in mineral oil (0.94 g, 24 mmol) under nitrogen. The reaction was warmed to 25° C. after 10 min, treated with 2,3-O-isopropylidene-D-erythronolactone (3.1 g, 20 mmol), and heated to 80° C. under nitrogen for 17 h. The reaction was cooled to 25° C., diluted with EtOAc (500 mL), washed with 10% hydrochloric acid solution (250 mL), water (250 mL), brine (250 mL), dried over MgSO₄, concentrated in vacuo and purified by silica gel chromatography (eluant: 5-10% MeOH/dichloromethane) to afford the title compound (5.3 g, 81%). MS: 317.1 (M−H).

### Step B: Synthesis of (4R,5S)-2,2-dimethyl-5-((naphthalen-2-ylsulfonyl)methyl)-1,3-dioxolane-4-carboxylic acid

A solution of (4R,5S)-2,2-dimethyl-5-((naphthalen-2-ylthio)methyl)-1,3-dioxolane-4-carboxylic acid (5.3 g, 17 mmol) in methanol (250 mL) and water (150 mL) was cooled to 0° C. and treated with Oxone monopersulfate (20 g, 33 mmol). The reaction mixture was allowed to warm to 25° C. over 1 h and stirred for a further 4 h. The reaction mixture was diluted with 10% hydrochloric acid solution (500 mL) and extracted with dichloromethane (3×500 mL). The combined organic layers were dried over MgSO₄ and concentrated in vacuo to afford the title compound (5.5 g, 86%) as a white solid. MS: 349.1 (M−H).

### Step C: Synthesis of (4R,5S)-2,2-dimethyl-5-((naphthalen-2-ylsulfonyl)methyl)-N-((R)-6-(piperidin-1-ylmethyl)-1,2,3,4-tetrahydronaphthalen-1-yl)-1,3-dioxolane-4-carboxamide

A solution of (4R,5S)-2,2-dimethyl-5-((naphthalen-2-ylsulfonyl)methyl)-1,3-dioxolane-4-carboxylic acid (1.5 g, 4.3 mmol) and (R)-6-(piperidin-1-ylmethyl)-1,2,3,4-tetrahydronaphthalen-1-amine (1.0 g, 4.3 mmol) in DMF (30 mL) was treated with 1-hydroxybenzotriazole (0.87 g, 6.4 mmol) and N-(3-dimethylaminopropyl)-NA-ethyl-carbodiimide hydrochloride (1.2 g, 6.4 mmol). The reaction was stirred at 23° C. under nitrogen for 3 h, diluted with EtOAc (200 mL), washed with saturated NaHCO₃ solution (100 mL), water (100 mL), brine (100 mL), dried over MgSO₄, concentrated in vacuo and purified by silica gel chromatography (eluant: 2-5% MeOH/dichloromethane), affording the title compound (2.03 g, 81%) as a white solid. MS: 577.3 (M+H).

### Step D: Synthesis of (2R,3S)-2,3-dihydroxy-4-(naphthalen-2-ylsulfonyl)-N-((R)-6-(piperidin-1-ylmethyl)-1,2,3,4-tetrahydronaphthalen-1-yl)butanamide

A solution of (4R,5S)-2,2-dimethyl-5-((naphthalen-2-ylsulfonyl)methyl)-N-((R)-6-(piperidin-1-ylmethyl)-1,2,3,4-tetrahydronaphthalen-1-yl)-1,3-dioxolane-4-carboxamide (2.3 g, 4 mmol) in methanol (53 mL) was treated with hydrogen chloride 4.0 m in 1,4-dioxane (7 mL, 28 mmol). The reaction was capped and stirred at 23° C. for 65 h and concentrated in vacuo to afford the title compound (2 g, 88%) as a white solid. MS: 537.2 (M+H).

### Example 8

### Synthesis of (R)-4-(naphthalen-2-ylsulfonyl)-N-(6-(piperidin-1-ylmethyl)-1,2,3,4-tetrahydronaphthalen-1-yl)butanamide

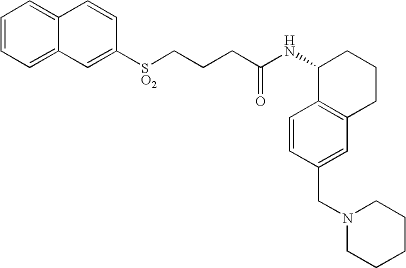

### Step A: Synthesis of 4-(naphthalen-2-ylthio)butanoic acid

A solution of 2-naphthalenethiol (5.6 g, 34 mmol) in DMF (30 mL) was cooled to 0° C. and treated with sodium hydride, 60% dispersion in mineral oil (1.6 g, 39 mmol) under nitrogen. After 15 min, γ-butyrolactone (2 g, 23 mmol) was added, and the reaction was warmed to 23° C. After a further 30 min, the reaction mixture was heated to 100° C. After 6 h, the solution was cooled to 23° C., diluted with ethyl acetate (200 mL), washed with 10% hydrochloric acid solution (2×100 mL), water (100 mL), brine (100 mL), dried over MgSO₄, concentrated in vacuo and purified by silica gel chromatography (eluant: 0-5% MeOH/dichloromethane) to afford 4-(naphthalen-2-ylthio)butanoic acid (3.5 g, 53%).

### Step B: Synthesis of 4-(naphthalen-2-ylsulfonyl)butanoic acid

A solution of 4-(naphthalen-2-ylthio)butanoic acid (2.3 g, 9.42 mmol) in methanol (50 mL), 1,4-dioxane (10 mL) and water (50 mL) was treated with Oxone monopersulfate (17 g, 28 mmol). The reaction mixture was stirred at 23° C. for 22 h. The reaction mixture was diluted with ethyl acetate (100 mL) and washed with 10% hydrochloric acid solution (100 mL), water (100 mL), brine (100 mL), dried over MgSO₄, concentrated in vacuo and purified by silica gel chromatography (eluant: 5% MeOH/dichloromethane) to afford the title compound (1.49 g, 57%). MS: 277.1 (M−H).

### Step C: Synthesis of (R)-4-(naphthalen-2-ylsulfonyl)-N-(6-(piperidin-1-ylmethyl)-1,2,3,4-tetrahydronaphthalen-1-yl)butanamide

A solution of 4-(naphthalen-2-ylsulfonyl)butanoic acid (205 mg, 0.73 mmol) and (R)-6-(piperidin-1-ylmethyl)-1,2,3,4-tetrahydronaphthalen-1-amine (182 mg, 0.74 mmol) in DMF (8 mL) was treated with HOBT (150 mg, 1.10 mmol) and EDCI (212 mg, 1.10 mmol). The reaction was stirred at 23° C. for 4 h, diluted with ethyl acetate (50 mL) and washed with saturated sodium bicarbonate solution (25 mL), water (25 mL), brine (25 mL), dried over MgSO₄, concentrated in vacuo and purified by silica gel chromatography (eluant: 4-8% MeOH/dichloromethane) to afford the title compound (260 mg, 70%). MS: 505.3 (M+H).

### Example 9

### Synthesis of (R)-2-hydroxy-4-(naphthalen-2-ylsulfonyl)-N-((R)-6-(piperidin-1-ylmethyl)-1,2,3,4-tetrahydronaphthalen-1-yl)butanamide

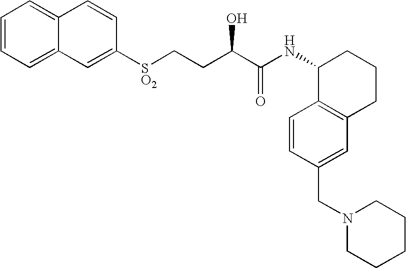

### Step A: Synthesis of (R)-2-hydroxy-4-(naphthalen-2-ylthio)butanoic acid

A solution of 2-naphthalenethiol (740 mg, 4.61 mmol) in DMF (20 mL) was cooled to 0° C. and treated with sodium hydride, 60% dispersion in mineral oil (200 mg, 4.99 mmol) under nitrogen. After 15 min, (R)-3-hydroxy-dihydrofuran-2(3H)-one (0.3 mL, 3.84 mmol) was added, and the reaction was warmed to 23° C. After 10 min, the reaction mixture was heated to 80° C. After 15 h, the solution was cooled to 23° C., diluted with ethyl acetate (100 mL), washed with 10% hydrochloric acid solution (75 mL), water (50 mL), brine (50 mL), dried over MgSO₄, concentrated in vacuo and purified by silica gel chromatography (eluant: 2-5% MeOH/dichloromethane, followed by 10% MeOH/dichloromethane+1% AcOH) to afford the title compound (125 mg, 12%). MS: 263.2 (M+H).

### Step B: Synthesis of (R)-2-hydroxy-4-(naphthalen-2-ylsulfonyl)butanoic acid.

A solution of (R)-2-hydroxy-4-(naphthalen-2-ylthio)butanoic acid (125 mg, 0.477 mmol) in methanol (2.5 mL), 1,4-dioxane (1 mL) and water (2.5 mL) was cooled to 0° C. and treated with Oxone monopersulfate compound (585 mg, 0.954 mmol). The reaction was stirred for 4 h, diluted with 10% hydrochloric acid solution (25 mL) and extracted with dichloromethane (3×25 mL). The combined organic layers were dried over MgSO₄ and concentrated in vacuo to afford the title compound (140 mg, 99%). MS: 295.1 (M+H).

### Step C: Synthesis of (R)-2-hydroxy-4-(naphthalen-2-ylsulfonyl)-N-((R)-6-(piperidin-1-yl-methyl)-1,2,3,4-tetrahydronaphthalen-1-yl)butanamide

A solution of (R)-2-hydroxy-4-(naphthalen-2-ylsulfonyl)butanoic acid (140 mg, 0.476 mmol) and (R)-6-(piperidin-1-ylmethyl)-1,2,3,4-tetrahydronaphthalen-1-amine (116 mg (0.476 mmol) in DMF (5 mL) was treated with HOBT (90 mg, 0.666 mmol) and EDCI (128 mg, 0.666 mmol). The reaction was stirred at 23° C. for 15 h, diluted with ethyl acetate (25 mL) and washed with saturated sodium bicarbonate solution (15 mL), water (15 mL), brine (15 mL), dried over MgSO₄, concentrated in vacuo and purified by silica gel chromatography (eluant: 4-8% MeOH/dichloromethane) to afford the title compound (165 mg, 67%). MS: 521.3 (M+H).

### Example 10

### Synthesis of (R)-2-methoxy-4-(naphthalen-2-ylsulfonyl)-N-((R)-6-(piperidin 1-ylmethyl)-1,2,3,4-tetrahydronaphthalen-1-yl)butanamide

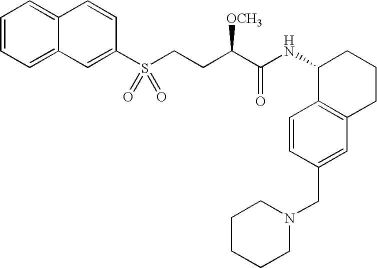

### Step A: Synthesis of (R)-3-methoxydihydrofuran-2(3H)-one

A solution of (R)-3-hydroxy-dihydrofuran-2(3H)-one (475 mg, 4.653 mmol) in acetonitrile (30 mL) was treated with iodomethane (1.449 mL, 23.3 mmol) and silver(I) oxide (1.294 g, 5.58 mmol) in a sealed tube and capped. The tube was stirred at 23° C. for 6 h, heated to 75° C. for 15 h, cooled to 23° C. and filtered through celite. The filtrate was concentrated, and purified on silica gel (eluant: 20-30% EtOAc/hexane) to afford the title compound (384 mg, 71%) as a colorless oil.

### Step B: Synthesis of (R)-2-methoxy-4-(naphthalen-2-ylthio)butanoic acid

A solution of (R)-3-methoxy-dihydrofuran-2(3H)-one (355 mg, 3.05 mmol) and naphthalene-2-thiol (538 mg, 3.4 mmol) in DMF (15 mL) was cooled to 0° C. under nitrogen and treated with sodium hydride, 60% dispersion in mineral oil (146 mg, 3.7 mmol). After 10 min, the reaction mixture was heated to 80° C. and stirred for 17 h. After cooling to 23° C., the reaction was diluted with EtOAc (100 mL), washed with 10% hydrochloric acid (50 mL), water (50 mL), brine (50 mL), dried over MgSO₄, concentrated in vacuo and purified by silica gel chromatography (eluant: 1-10% methanol/dichloromethane) to afford the title compound (553 mg, 63%) as an yellow solid. MS: 277.2 (M+H).

### Step C: Synthesis of (R)-2-methoxy-4-(naphthalen-2-ylsulfonyl)butanoic acid

A solution of (R)-2-methoxy-4-(naphthalen-2-ylthio)butanoic acid (491 mg, 1.777 mmol) in methanol/water (20 mL, 1:1) was cooled to 0° C. and treated with Oxone monopersulfate (2.185 g, 3.55 mmol). The reaction mixture was allowed to warm to 23° C. over 3 h, diluted with EtOAc (50 mL) and washed with 10% hydrochloric acid (50 mL). The aqueous layer was further extracted with EtOAc (50 mL). The combined organic layers were washed with brine (50 mL), dried over MgSO₄ and concentrated in vacuo to afford the title compound (536 mg, 95%) as a yellow solid. MS: 309.1 (M+H).

### Step D: Synthesis of (R)-2-methoxy-4-(naphthalen-2-ylsulfonyl)-N-((R)-6-(piperidin-1-yl-methyl)-1,2,3,4-tetrahydronaphthalen-1-yl)butanamide

A solution of (R)-2-methoxy-4-(naphthalen-2-ylsulfonyl)butanoic acid (167 mg, 542 μmol) and (R)-6-(piperidin-1-ylmethyl)-1,2,3,4-tetrahydronaphthalen-1-amine (132 mg, 542 μmol) in DMF (5 mL) was treated with 1-hydroxybenzotriazole (110 mg, 0.812 mmol) and N-(3-dimethylaminopropyl)-N′-ethylcarbodiimide hydrochloride (156 mg, 0.812 mmol). The reaction was stirred under nitrogen at 23° C. for 5 h, diluted with EtOAc (40 mL), washed with saturated NaHCO₃ solution (25 mL), water (25 mL), brine (25 mL), dried over MgSO₄, concentrated in vacuo and purified by silica gel chromatography (eluant: 5% methanol/dichloromethane), affording the title compound (225 mg, 75%) as a crystalline white solid. MS: 535.2 (M+H).

### Example 11

### Synthesis of (S)-2-hydroxy-4-(naphthalen-2-ylsulfonyl)-N-((R)-6-(piperidin-1-ylmethyl)-1,2,3,4-tetrahydronaphthalen-1-yl)butanamide

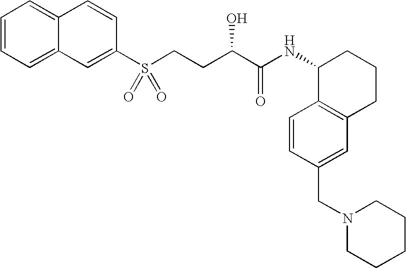

### Step A: Synthesis of (S)-2-hydroxy-4-(naphthalen-2-ylthio)butanoic acid

A solution of (S)-(−)-α-hydroxy-γ-butyrolactone (505 mg, 4.947 mmol) and naphthalene-2-thiol (872 mg, 5.4 mmol) in DMF (20 mL) was cooled to 0° C. under nitrogen, and treated with sodium hydride, 60% dispersion in mineral oil (416 mg, 10 mmol). After 10 minutes, the reaction was heated to 80° C. for 14 h, cooled to 23° C., diluted with EtOAc (100 mL) and washed with 10% hydrochloric acid solution (50 mL) and brine (50 mL). The organic layer was dried over MgSO₄, concentrated in vacuo and purified using silica gel. chromatography (eluant: 2-5-10% methanol/dichloromethane), affording the title compound (274 mg, 21%) as a yellow solid. MS: 261.1 (M−H).

### Step B: Synthesis of (S)-2-hydroxy-4-(naphthalen-2-ylsulfonyl)butanoic acid

A solution of (S)-2-hydroxy-4-(naphthalen-2-ylthio)butanoic acid (250 mg, 0.953 mmol) in methanol/water (1:1, 20 mL) was cooled to 0° C. and treated with Oxone monopersulfate (1.172 g, 1.91 mmol). The reaction was allowed to warm to 23° C. over 2 h, diluted with 10% hydrochloric acid solution (50 mL) and extracted with dichloromethane (3×50 mL). The combined organic layers were dried over MgSO₄ and concentrated in vacuo to afford the title compound (218 mg, 69%) as a white solid.

### Step C: Synthesis of (S)-2-hydroxy-4-(naphthalen-2-ylsulfonyl)-N-((R)-6-(piperidin-1-yl-methyl)-1,2,3,4-tetrahydronaphthalen-1-yl)butanamide

A solution of(S)-2-hydroxy-4-(naphthalen-2-ylsulfonyl)butanoic acid (182 mg, 0.618 mmol) and (R)-6-(piperidin-1-ylmethyl)-1,2,3,4-tetrahydronaphthalen-1-amine (151 mg, 0.618 mmol) in DMF (5 mL) was treated with 1-hydroxybenzotriazole (125 mg, 0.928 mmol) and N-(3-dimethylaminopropyl)-NM-ethylcarbodiimide hydrochloride (178 mg, 0.928 mmol). The reaction was stirred at 23° C. under nitrogen for 3 h, diluted with EOAc (30 mL), washed with saturated NaHCO₃ solution (25 mL), water (25 mL), brine (25 mL), dried over MgSO₄, concentrated in vauo and purified by silica gel chromatography (eluant: 10% methanol/dichloromethane) affording the title compound (234 mg, 69%) as an yellow solid. MS: 521.2 (M+H).

### Example 12

### Synthesis of 3-hydroxy-3-(1-(naphthalen-2-ylsulfonyl)cyclopropyl)-N-((R)-6-(piperidin-1-ylmethyl)-1,2,3,4-tetrahydronaphthalen-1-yl)propanamide

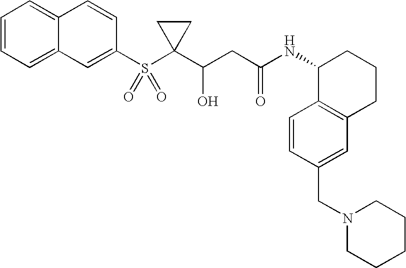

### Step A: Synthesis of methyl 2-(naphthalen-2-ylthio)acetate

A suspension of potassium carbonate (10 g, 76 mmol), naphthalene-2-thiol (11 g, 69 mmol) and methyl 2-bromoacetate (12 g, 76 mmol) in 10 mL of DMF was stirred overnight. The reaction mixture was filtered with the help of large excess of DCM. The filtrate was evaporated to dryness. Flash chromatography (SiO₂, hexane to hexane/DCM=2:1 to 1:1 to pure DCM) afforded the title compound (14.6 g, 63 mmol, 92% yield) as a white solid.

### Step B: Synthesis of methyl 2-(naphthalen-2-ylsulfonyl)acetate

To a suspension of methyl 2-(naphthalen-2-ylthio)acetate (4.64 g, 20 mmol) in MeOH/H₂O (70/50 mL) at RT was added Oxone(R) monopersulfate (22 mL, 40 mmol). After 2 h, MeOH was removed under vacuum and the residue was extracted with DCM. Flash chromatography (SiO₂, DCM to DCM/EtOAc=4:1) gave the title compound (4.58 g) as a white solid.

### Step C: Synthesis of methyl 1-(naphthalen-2-ylsulfonyl)cyclopropane carboxylate

A mixture of potassium carbonate (0.904 mL, 15.0 mmol), 1,2-dibromoethane (0.904 mL, 10.5 mmol) and methyl 2-(naphthalen-2-ylsulfonyl)acetate (1.98 g, 7.49 mmol) in DMF (3 mL) was stirred at 60° C. for 5 h. After cooling to RT, the reaction was diluted with EtOAc/hexane=2:1 and washed with water, dried over Na₂SO₄ and evaporated to dryness. CC (SiO₂, DCM) gave the title compound (2.08 g, 95.6% yield) as a white solid.

### Step D: Synthesis of 1-(naphthalen-2-ylsulfonyl)cyclopropane carbaldehyde

To a solution of methyl 1-(naphthalen-2-ylsulfonyl)cyclopropane carboxylate (1.90 g, 0.654 mmol) in dry DCM (30 mL) at −78° C. was added dropwise diisobutylaluminum (1.396 g, 9.816 mmol) (9.82 mL of 1 M solution in hexanes). After 1 h, MeOH (5 mL) was added and the cold bath was removed. Na₂SO₄.10H₂O (15 g) and water (2 mL) were added and the mixture was stirred at RT for 4 h. The suspension was filtered with the help of EtOAc and the solvent was evaporated. Column chromatograph (SiO₂, DCM to DCM/EtOAc=2:1) gave the title compound (1.6 g, 94% yield) as a white solid and (1-(naphthalen-2-ylsulfonyl)-cyclopropyl)methanol (50 mg, 2.9% yield).

### Step E: Synthesis of (E)-methyl 3-(1-(naphthalen-2-ylsulfonyl)cyclopropyl)acrylate

A solution of 1-(naphthalen-2-ylsulfonyl)cyclopropane carbaldehyde (700 mg, 2.69 mmol) and diisobutylaluminum hydride (1.798 g, 5.378 mmol) in DCM was stirred at RT overnight. The reaction mixture was concentrated and directed submitted to flash chromatograph (SiO₂, DCM) to give the title compound (840 mg, 98.7% yield) as a white solid.

### Step F: Synthesis of (E)-3-(1-(naphthalen-2-ylsulfonyl)cyclopropyl)acrylic acid

A solution of (E)-methyl 3-(1-(naphthalen-2-ylsulfonyl)cyclopropyl)acrylate (270 mg, 0.853 mmol) and 4 N HCl (10 mL) solution in dioxane was refluxed for 4 h. The solution was cooled to RT and the solvent was removed. The residue was submitted to column chromatography (SiO₂, DCM to EtOAc) to afford the title compound (250 mg, 96.9% yield) as a white solid.

### Step F: Synthesis of (R,E)-3-(1-(naphthalen-2-ylsulfonyl)cyclopropyl)-N-(6-(piperidin-1-yl-methyl)-1,2,3,4-tetrahydronaphthalen-1-yl)acrylamide

A solution of 1-(3-dimethylaminopropyl)-3-ethylcarbodiimide hydrochloride (0.794 mmol), 1-hydroxybenzotriazole (0.794 mmol), (E)-3-(1-(naphthalen-2-ylsulfonyl)-cyclopropyl)-acrylic acid (0.794 mmol) and (R)-6-(piperidin-1-ylmethyl)-1,2,3,4-tetrahydronaphthalen-1-amine (0.873 mmol) in DMF (1.5 mL) was stirred overnight. The reaction was quenched with sat. NaHCO₃, extracted with EtOAc/hexane=3:1, washed with brine, dried over Na₂SO₄, and evaporated. Column chromatography (SiO₂, EtOAc to EtOAc/MeOH=100:5 to 100:10 to 100:15) afforded the title compound (340 mg, 81.0% yield) as a white solid.

### Step G: Synthesis of 2,3-dihydroxy-3-(1-(naphthalen-2-ylsulfonyl)cyclopropyl)-N-((R)-6-(piperidin-1-ylmethyl)-1,2,3,4-tetrahydronaphthalen-1-yl)propanamide

To a solution of (R,E)-3-(1-(naphthalen-2-ylsulfonyl)cyclopropyl)-N-(6-(piperidin-1-ylmethyl)-1,2,3,4-tetrahydronaphthalen-1-yl)acrylamide (100 mg, 0.189 mmol) in t-BuOH/THF/H₂O (13 mL, 10:2:1) was added 4-methylmorpholine 4-oxide (44 mg, 0.378 mmol), followed by OSO₄ (57.6 mg of OSO₄ 2.5% in tBuOH). The reaction mixture was stirred at room temperature over 5 h. The solution was concentrated and diluted with EtOAc, washed with brine, dried and evaporated to dryness. Column chromatography (SiO₂, EtOAc to EtOAc/MeOH=100:5 to 100:10 to 100:15) afforded the title compound (35 mg, 33% yield) as a white solid.

### Step H: Synthesis of methyl 3-(1-(naphthalen-2-ylsulfonyl)cyclopropyl)propanoate

A suspension of palladium (8.41 mg, 79.0 μmol) (80 mg of 10% Pd/C) and (E)-methyl 3-(1-(naphthalen-2-ylsulfonyl)cyclopropyl)acrylate (250 mg, 790 μmol) in 20 mL of EtOAc was stirred under H₂ for 18 h. The reaction mixture was evaporated to dryness and was submitted to column chromatography (SiO₂, DCM to DCM/EtOAc=1:1) to give the title compound as a white solid.

### Step I: Synthesis of 3-(1-(naphthalen-2-ylsulfonyl)cyclopropyl)propanoic acid

A solution of methyl 3-(1-(naphthalen-2-ylsulfonyl)cyclopropyl)propanoate (220 mg, 0.691 mmol) in 15 mL of dioxane and 5 mL of 10% HCl/water was refluxed for 2 h. Dioxane was evaporated and the residue was extracted by EtOAc, washed with water and dried over Na₂SO₄. The EtOAc was evaporated to give the title compound (180 mg, 85.6% yield) as a white solid.

### Step J: Synthesis of (R)-3-(1-(naphthalen-2-ylsulfonyl)cyclopropyl)-N-(6-(piperidin-1-ylmethyl)-1,2,3,4-tetrahydronaphthalen-1-yl)propanamide

A solution of 1-(3-dimethylaminopropyl)-3-ethylcarbodiimide hydrochloride (0.657 mmol), 1-hydroxybenzotriazole (0.657 mmol), 3-(1-(naphthalen-2-ylsulfonyl)cyclopropyl)-propanoic acid (0.657 mmol) and (R)-6-(piperidin-1-ylmethyl)-1,2,3,4-tetrahydronaphthalen-1-amine (0.789 mmol) in DMF (1.5 mL) was stirred overnight. The reaction was quenched with sat. NaHCO₃, extracted with EtOAc/hexane (4:1), washed with brine and dried over Na₂SO₄. CC (SiO₂, EtOAc to EtOAc/2M NH₃ in MeOH=100:3 to 100:5 to 100:8) gave the title compound (210 mg, 396 μmol, 60.2% yield) as a sticky oil.

### Step K: Synthesis of tert-butyl 3-hydroxy-3-(1-(naphthalen-2-ylsulfonyl)cyclopropyl)-propanoate

To a solution of LDA (2.0 M in heptane/THF/Ethylbenzene, 4.8 mL, 9.6 mmol) in dry THF (10 mL) at −78° C. was added t-butyl acetate (0.992 mL, 7.683 mmol) and the solution was stirred at 0° C. for 1 h. To this solution at −78° C. was added 1-(naphthalen-2-ylsulfonyl)-cyclopropane-carbaldehyde (500 mg, 1.921 mmol) in 10 mL of dry THF and the reaction was stirred at −78° C. for 15 min. The reaction was quenched with sat. NH₄Cl and extracted with EtOAc, dried over Na₂SO₄ and evaporated. Column chromatography (SiO₂, DCM to DCM/EtOAc=10:1 to 100:15) gave the title compound (710 mg, 98% yield) as a white solid.

### Step L: Synthesis of 3-hydroxy-3-(1-(naphthalen-2-ylsulfonyl)cyclopropyl)propanoic acid

To a solution of tert-butyl 3-hydroxy-3-(1-(naphthalen-2-ylsulfonyl)cyclopropyl)-propanoate (530 mg, 1.408 mmol) in DCM (15 mL) was added trifluoroethanoic acid (1.464 mL, 19.710 mmol). After stirring at RT overnight, the solvent was evaporated to dryness under high vacuum and the residue was submitted to column chromatography (SiO₂, DCM/EtOAc=1:1 to pure EtOAc) to give the title compound (431 mg, 95.6% yield) as a white solid.

### Step M: Synthesis of 3-hydroxy-3-(1-(naphthalen-2-ylsulfonyl)cyclopropyl)-N-((R)-6-(piperidin-1-ylmethyl)-1,2,3,4-tetrahydronaphthalen-1-yl)propanamide

A solution of N¹-((ethylimino)methylene)-N³,N³-dimethylpropane-1,3-diamine hydrochloride (132 mg, 0.687 mmol), 1H-benzo[d][1,2,3]triazol-1-ol (92.8 mg, 0.687 mmol), 3-hydroxy-3-(1-(naphthalen-2-ylsulfonyl)cyclopropyl)propanoic acid (220 mg, 0.687 mmol) and (R)-6-(piperidin-1-ylmethyl)-1,2,3,4-tetrahydronaphthalen-1-amine (201 mg, 0.824 mmol) in DMF (1 mL) was stirred at RT over the weekend. The reaction was quenched with sat. NaHCO₃, extracted with EtOAc, washed with brine, dried over Na₂SO₄ and evaporated to dryness. CC (SiO₂, EtOAc to EtOAc/2M NH3 in MeOH=100:3 to 100:5 to 100:7 to 100:12) gave the title compound (165 mg, 302 μmol, 43.9% yield) as a sticky oil.

### Example 13

### Synthesis of 2-(naphthalen-2-ylsulfonyl)-5-oxo-5-((R)-6-(piperidin-1-ylmethyl)-1,2,3,4-tetrahydronaphthalen-1-ylamino)pentanoic acid

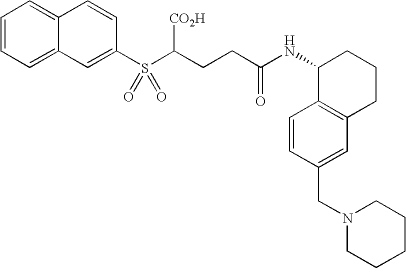

### Step A: Synthesis of 5-tert-butyl 1-methyl 2-(naphthalen-2-ylsulfonyl)pentanedioate

A suspension of potassium carbonate (0.30 mL, 5.0 mmol), tert-butyl acrylate (0.73 mL, 5.0 mmol) and methyl 2-(naphthalen-2-ylsulfonyl)acetate (1.3 g, 5.0 mmol) in DMF (2 mL) was stirred at RT for 2 h. The reaction mixture was diluted with hexane/EtOAc=5:1, washed with water, dried over Na₂SO₄ and evaporated to dryness. CC (SiO₂, DCM) afforded 5-tert-butyl 1-methyl 2-(naphthalen-2-ylsulfonyl)pentanedioate (220 mg) with 90% purity as an colorless oil, together with 5-tert-butyl 1-methyl 2-(naphthalen-2-ylsulfonyl)pentanedioate (2.0 g) with about 75% purity (the minor product is 1,5-di-tert-butyl 3-methyl 3-(naphthalen-2-ylsulfonyl)pentane-1,3,5-tricarboxylate).

### Step B: Synthesis of 5-methoxy-4-(naphthalen-2-ylsulfonyl)-5-oxopentanoic acid

To a solution of 5-tert-butyl 1-methyl 2-(naphthalen-2-ylsulfonyl)pentanedioate (1.2 g, 3.1 mmol) in DCM (20 mL) was added 2,2,2-trifluoroacetic acid (1.4 mL, 18 mmol) and the reaction mixture was stirred at RT for 14 h. The solvent and TFA was evaporated under vacuum. Column chromatography (SiO₂, DCM to DCM/EtOAc=5:1 to 2:1 to pure EtOAc) gave 5-methoxy-4-(naphthalen-2-ylsulfonyl)-5-oxopentanoic acid (0.68 g, 2.0 mmol, 66% yield) as a white solid and 4-(methoxycarbonyl)-4-(naphthalen-2-ylsulfonyl)heptanedioic acid (0.12 g, 0.29 mmol, 9.6% yield) as a white solid.

### Step C: Synthesis of methyl 2-(naphthalen-2-ylsulfonyl)-5-oxo-5-((R)-6-(piperidin-1-ylmethyl)-1,2,3,4-tetrahydronaphthalen-1-ylamino)pentanoate

A solution of N1-((ethylimino)methylene)-N3,N3-dimethylpropane-1,3-diamine hydrochloride (365 mg, 1.903 mmol), 1H-benzo[d][1,2,3]triazol-1-ol (257 mg, 1.903 mmol), 5-methoxy-4-(naphthalen-2-ylsulfonyl)-5-oxopentanoic acid, and (R)-6-(piperidin-1-ylmethyl)-1,2,3,4-tetrahydronaphthalen-1-amine (558 mg, 2.283 mmol) in DMF (1 mL) was stirred at RT over the weekend. The reaction mixture was quenched with sat. NaHCO₃, extracted with EtOAc, washed with brine, dried over Na₂SO₄ and evaporated to dryness. Column chromatography (SiO₂, EtOAc to EtOAc/2M NH₃ in MeOH=100:3 to 100:5 to 100:7 to 100:10) gave methyl (950 mg, 1688 μmol, 88.7% yield) as a white solid.

### Step D: Synthesis of 2-(naphthalen-2-ylsulfonyl)-5-oxo-5-((R)-6-(piperidin-1-yl-methyl)-1,2,3,4-tetrahydronaphthalen-1-ylamino)pentanoic acid

A solution of lithium hydroxide hydrate (107 mg, 2.559 mmol) and methyl 2-(naphthalen-2-ylsulfonyl)-5-oxo-5-((R)-6-(piperidin-1-ylmethyl)-1,2,3,4-tetrahydronaphthalen-1-ylamino)pentanoate in MeOH/THF/H₂O (5 mL, 5/8/2) was stirred at RT for 2 h. The majority of the solvent was evaporated and the residue was directly submitted to flash chromatography (EtOAc/2 M NH₃ in MeOH=100:10 to 100:20 to 100:30 to 100:50) to give the title compound (410 mg, 87.6% yield) as a white solid.

### Example 14

### Synthesis of (2R,3S)-N-((R)-6-((2,6-dimethylpiperidin-1-yl)methyl)-1,2,3,4-tetrahydro-naphthalen-1-yl)-2,3-dihydroxy-4-(naphthalen-2-ylsulfonyl)butanamide

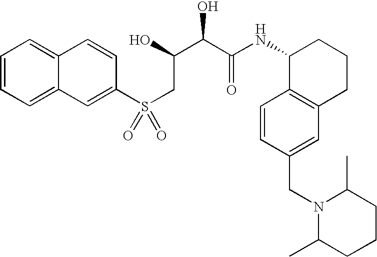

### Step A: Synthesis of (4R,5S)-N-((R)-6-(hydroxymethyl)-1,2,3,4-tetrahydronaphthalen-1-yl)-2,2-dimethyl-5-((naphthalen-2-ylsulfonyl)methyl)-1,3-dioxolane-4-carboxamide

(R)-(5-Amino-5,6,7,8-tetrahydronaphthalen-2-yl)methanol (0.33 g, 1.85 mmol) and (4R,5S)-2,2-dimethyl-5-((naphthalen-2-ylsulfonyl)methyl)-1,3-dioxolane-4-carboxylic acid (0.5 g, 1.42 mmol) were dissolved in DMF (2 mL). EDCI 0.42 g, 2.2 mmol) and HOBT (0.3 g) were added and the reaction mixture was stirred at room temp. overnight. The reaction mixture is quenched with sat. sodium bicarbonate, extracted with ethyl acetate, washed with brine, washed with water, dried with sodium sulfate, and concentrated. The crude product was purified on silica using 0 to 5% MeOH in dichloromethane to give the title compound (410 mg, 54%).

### Step B: Synthesis of (4R,5S)-N-((R)-6-formyl-1,2,3,4-tetrahydronaphthalen-1-yl)-2,2-dimethyl-5-((naphthalen-2-ylsulfonyl)methyl)-1,3-dioxolane-4-carboxamide

(4R,5S)-N-((R)-6-(Hydroxymethyl)-1,2,3,4-tetrahydronaphthalen-1-yl)-2,2-dimethyl-5-((naphthalen-2-ylsulfonyl)methyl)-1,3-dioxolane-4-carboxamide (400 mg) was dissolved in DCM and manganese oxide (0.7 g) was added. The reaction mixture was stirred overnight at room temp. then filtered through celite. The celite pad was then washed repeatedly with methanol and the reaction mixture was then concentrated. The crude product was then purified on silica using 0 to 100% ethyl acetate in hexane to give the title compound (210 mg, 53%).

### Step C: Synthesis of (4R,5S)-N-((R)-6-((2,6-dimethylpiperidin-1-yl)methyl)-1,2,3,4-tetrahydronaphthalen-1-yl)-2,2-dimethyl-5-((naphthalen-2-ylsulfonyl)methyl)-1,3-dioxolane-4-carboxamide

A solution of (4R,5S)-N-((R)-6-formyl-1,2,3,4-tetrahydronaphthalen-1-yl)-2,2-dimethyl-5-((naphthalen-2-ylsulfonyl)methyl)-1,3-dioxolane-4-carboxamide (150 mg), 2,6-dimethylpiperidine (200 mg), sodium triacetoxy borohydride (125 mg), and two drops of acetic acid in dichloroethane (2 mL) was stirred at room temp. overnight. The reaction mixture was quenched with sat. sodium bicarbonate. (10 mL), and extracted with ethyl acetate (2×25 mL). The extracts are washed with brine (10 mL), dried with sodium sulfate, and concentrated. The crude product was purified on silica using 0 to 5% methanol in DCM to give the title compound (90 mg, 50%).

### Step D: Synthesis of (2R,3S)-N-((R)-6-((2,6-dimethylpiperidin-1-yl)methyl)-1,2,3,4-tetrahydronaphthalen-1-yl)-2,3-dihydroxy-4-(naphthalen-2-ylsulfonyl)butanamide

To a solution of (4R,5S)-N-((R)-6-((2,6-dimethylpiperidin-1-yl)methyl)-1,2,3,4-tetrahydronaphthalen-1-yl)-2,2-dimethyl-5-((naphthalen-2-ylsulfonyl)methyl)-1,3-dioxolane-4-carboxamide (90 mg) in methanol (10 mL) was added 10 eq. of 4 M HCl in dioxane. The reaction mixture was allowed to stir overnight, then concentrated, dissolved in ethyl acetate, washed with sat. sodium bicarbonate, dried with sodium sulfate and concentrated again to give the title compound (60 mg, 71%). MS (ESI, pos. ion) m/z: 565.2 (M+1).

### Example 15

### Synthesis of (2R,3S)-N-((R)-6-(1-(tert-butylamino)ethyl)-1,2,3,4-tetrahydronaphthalen-1-yl)-2,3-dihydroxy-4-(3-(trifluoromethyl)phenylsulfonyl)butanamide

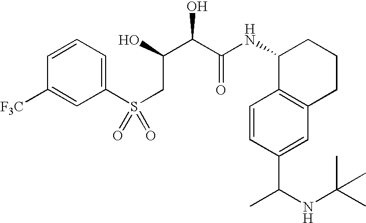

### Step A: Synthesis of (4R,5S)-2,2-dimethyl-5-((3-(trifluoromethyl)phenylsulfonyl)-methyl)-1,3-dioxolane-4-carboxylic acid

(2R,3S)-2,3-dihydroxy-4-(3-(trifluoromethyl)phenylsulfonyl)butanoic acid (140 mg), (1R)-6-(1-(tert-butylamino)ethyl)-1,2,3,4-tetrahydronaphthalen-1-amine (146 mg), and TEA (93 mg) were dissolved in dichloroethane (10 mL). HOBT and EDCI (1.3 equiv) were added and the reaction mixture was stirred overnight. The reaction mixture was quenched with sat. sodium bicarbonate, extracted with ethyl acetate, washed with brine, washed with water, and dried with sodium sulfate, and concentrated. The crude product was then purified on silica with 0 to 5% MeOH in DCM to give the title compound.

### Step B: Synthesis of (2R,3S)-N-((R)-6-(1-(tert-butylamino)ethyl)-1,2,3,4-tetrahydro-naphthalen-1-yl)-2,3-dihydroxy-4-(3-(trifluoromethyl)phenylsulfonyl)butanamide

(4R,5S)-N-((R)-6-(1-(tert-butylamino)ethyl)-1,2,3,4-tetrahydronaphthalen-1-yl)-2,2-dimethyl-5-((3-(trifluoromethyl)phenylsulfonyl)methyl)-1,3-dioxolane-4-carboxamide (90 mg, 0.151 mmol) was dissolved in MeOH, and 10 eq. of HCl (4M in dioxane) was added. The reaction mixture was capped and stirred overnight, then concentrated to give the title compound. MS (ESI, pos. ion) m/z: 557.2 (M+1).

Proceeding as described in examples above, the following compounds were prepared.

| Cpd. | | |
|---|---|---|
| # | Structure | Name |
| 1 | 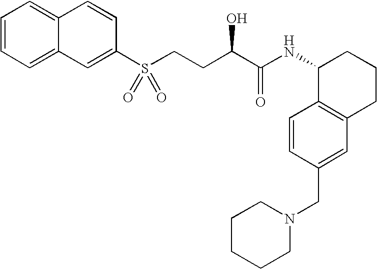 | (2R)-2-hydroxy-4-(2-naph- thalenyl-sulfonyl)-N-((1R)-6-(1-pipe- ridinylmethyl)-1,2,3,4-tetrahydro-1-naph- thalenyl)-butanamide |
| 2 | 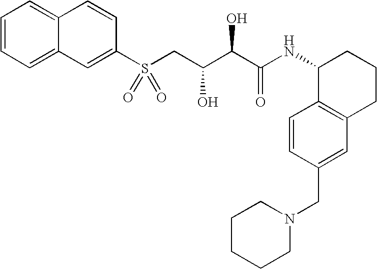 | (2R,3S)-2,3-dihydroxy-4-(naphthalen-2-yl- sulfonyl)-N-((R)-6-(piperidin-1-ylmethyl)-1,2,3,4-tetra- hydronaphthalen-1-yl)-butanamide |
| 3 | 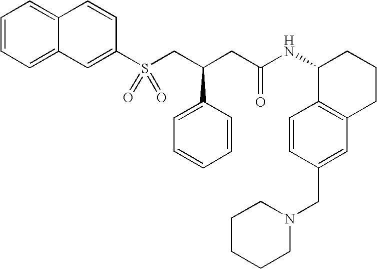 | (S)-4-(naphthalen-2-ylsulfonyl)-3-phenyl-N-((R)-6-(pipe- ridin-1-ylmethyl)-1,2,3,4-tetrahydro- naphthalen-1-yl)butanamide |
| 4 | 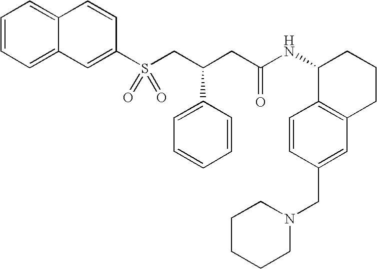 | (R)-4-(naphthalen-2-ylsulfonyl)-3-phenyl-N-((R)-6-(pipe- ridin-1-ylmethyl)-1,2,3,4-tetrahydro- naphthalen-1-yl)butanamide |
| 5 | 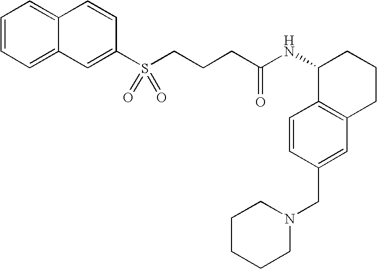 | 4-(2-naphthalenylsulfonyl)-N-((1R)-6-(1-pipe- ridinylmethyl)-1,2,3,4-tetrahydro-1-naph- thalenyl)butanamide |
| 6 | 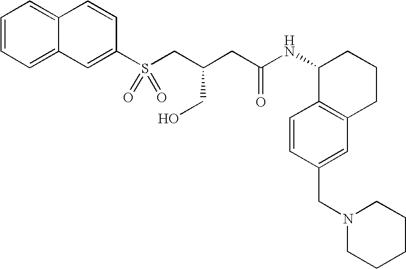 | (R)-3-(hydroxymethyl)-4-(naphthalen-2-yl- sulfonyl)-N-((R)-6-(piperidin-1-ylmethyl)-1,2,3,4-tetra- hydronaphthalen-1-yl)-butanamide |
| 7 | 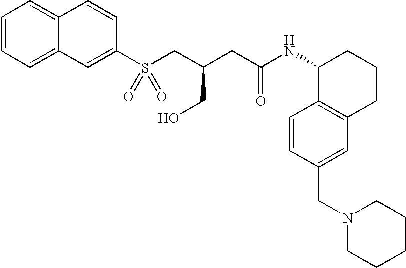 | (S)-3-(hydroxymethyl)-4-(naphthalen-2-yl- sulfonyl)-N-((R)-6-(piperidin-1-ylmethyl)-1,2,3,4-tetra- hydronaphthalen-1-yl)-butanamide |
| 8 | 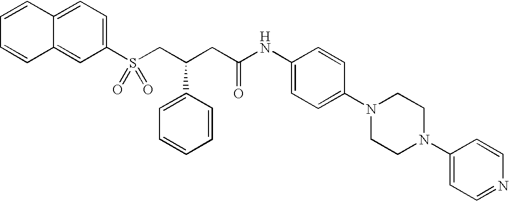 | (R)-4-(naphthalen-2-ylsulfonyl)-3-phenyl-N-(4-(4-(py- ridin-4-yl)piperazin-1-yl)-phe- nyl)butanamide |
| 9 | 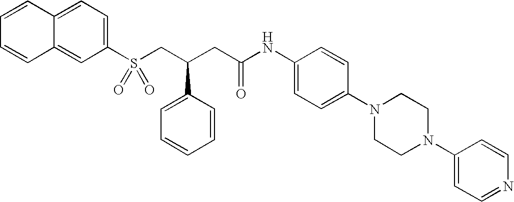 | (S)-4-(naphthalen-2-ylsulfonyl)-3-phenyl-N-(4-(4-(py- ridin-4-yl)piperazin-1-yl)phenyl)-butanamide |
| 10 | 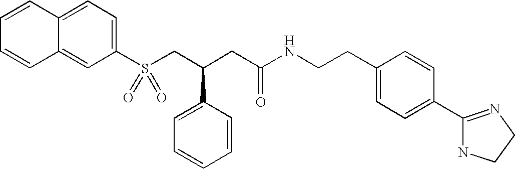 | (S)-N-(4-(4,5-dihydro-1H-imidazol-2-yl)phen- ethyl)-4-(naphthalen-2-ylsulfonyl)-3-phenyl- butanamide |
| 11 | 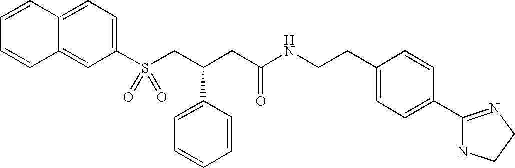 | (R)-N-(4-(4,5-dihydro-1H-imidazol-2-yl)-phen- ethyl)-4-(naphthalen-1-ylsulfonyl)-3-phenyl- butanamide |
| 12 | 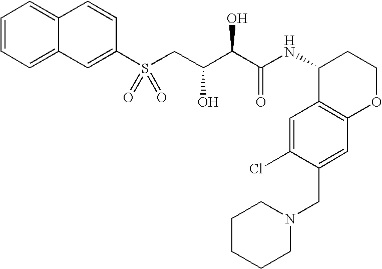 | (2R,3S)-N-((R)-6-chloro-7-(piperidin-1-yl-meth- yl)chroman-4-yl)-2,3-dihydroxy-4-(naph- thalen-2-ylsulfonyl)butanamide |
| 13 | 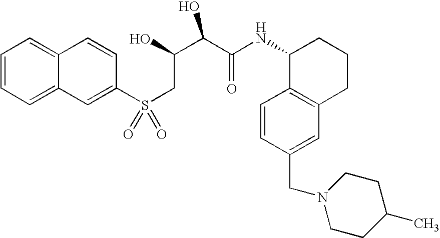 | (2R,3S)-2,3-dihydroxy-N-((R)-6-((4-methyl- piperidin-1-yl)methyl)-1,2,3,4-tetrahydro- naphthalen-1-yl)-4-(naphthalen-2-yl- sulfonyl)butanamide |
| 14 | 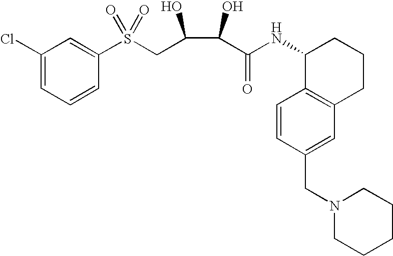 | (2R,3S)-4-(3-chlorophenylsulfonyl)-2,3-di- hydroxy-N-((R)-6-(piperidin-1-ylmethyl)-1,2,3,4-tetra- hydronaphthalen-1-yl)-butanamide |
| 15 | 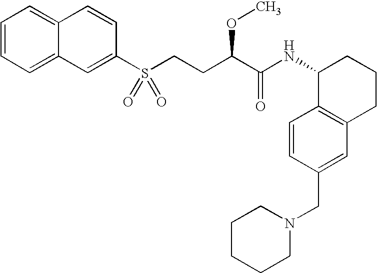 | (R)-2-methoxy-4-(naph- thalen-2-ylsulfonyl)-N-((R)-6-(pipe- ridin-1-ylmethyl)-1,2,3,4-tetra- hydronaphthalen-1-yl)butanamide |
| 16 | 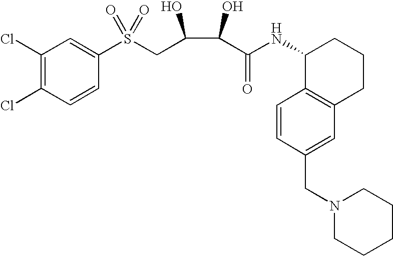 | (2R,3S)-4-(3,4-dichlorophenylsulfonyl)-2,3-di- hydroxy-N-((R)-6-(piperidin-1-ylmethyl)-1,2,3,4-tetra- hydronaphthalen-1-yl)-butanamide |
| 17 | 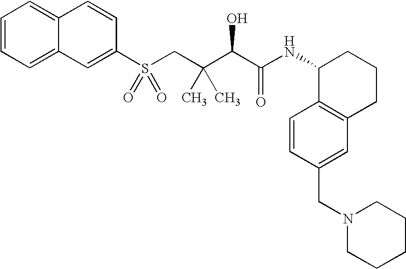 | (R)-2-hydroxy-3,3-dimethyl-4-(naphthalen-2-yl- sulfonyl)-N-((R)-6-(piperidin-1-ylmethyl)-1,2,3,4-tetra- hydronaphthalen-1-yl)butanamide |
| 18 | 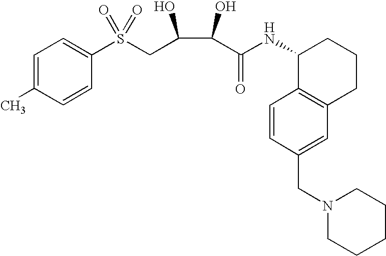 | (2R,3S)-2,3-dihydroxy-N-((R)-6-(piperidin-1-yl- methyl)-1,2,3,4-tetrahydronaphthalen-1-yl)-4-tosyl- butanamide |
| 19 | 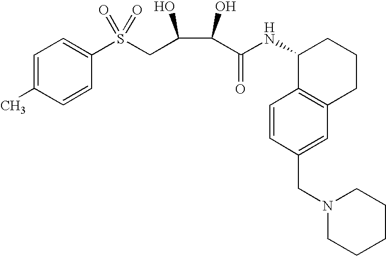 | (2R,3S)-2,3-dihydroxy-N-((R)-6-(piperidin-1-yl- methyl)-1,2,3,4-tetrahydronaphthalen-1-yl)-4-tosyl- butanamide |
| 20 | 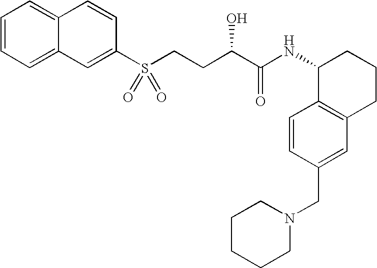 | (S)-2-hydroxy-4-(naph- thalen-2-ylsulfonyl)-N-((R)-6-(pipe- ridin-1-ylmethyl)-1,2,3,4-tetra- hydronaphthalen-1-yl)butanamide |
| 21 | 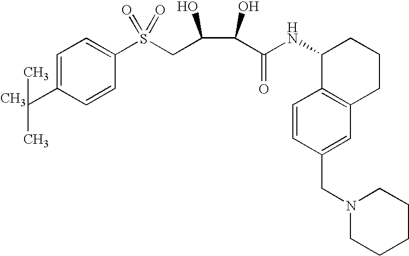 | (2R,3S)-4-(4-tert-butylphenylsulfonyl)-2,3-di- hydroxy-N-((R)-6-(piperidin-1-ylmethyl)-1,2,3,4-tetra- hydronaphthalen-1-yl)butanamide |
| 22 | 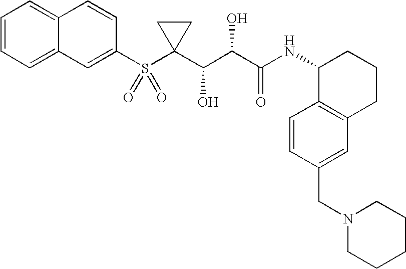 | (2S,3S)-2,3-dihydroxy-3-(1-(naphthalen-2-yl- sulfonyl)cyclopropyl)-N-((R)-6-(piperidin-1-yl- methyl)-1,2,3,4-tetrahydronaphthalen-1-yl)pro- panamide |
| 23 | 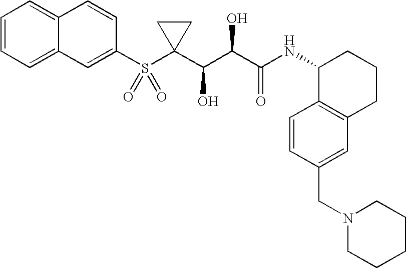 | (2R,3R)-2,3-dihydroxy-3-(1-(naphthalen-2-yl- sulfonyl)cyclopropyl)-N-((R)-6-(piperidin-1-yl- methyl)-1,2,3,4-tetrahydronaphthalen-1-yl)pro- panamide |
| 24 | 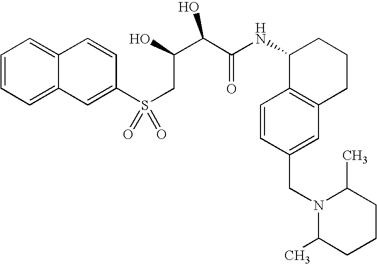 | (2R,3S)-N-((R)-6-((2,6-dimethylpiperidin-1-yl)meth- yl)-1,2,3,4-tetrahydronaphthalen-1-yl)-2,3-di- hydroxy-4-(naphthalen-2-yl- sulfonyl)butanamide |
| 25 | 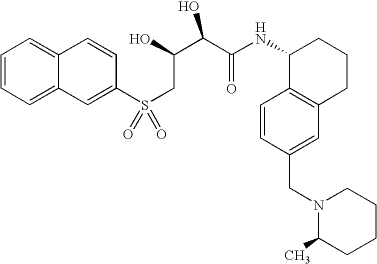 | (2R,3S)-2,3-dihydroxy-N-((R)-6-(((R)-2-meth- ylpiperidin-1-yl)methyl)-1,2,3,4-tetra- hydronaphthalen-1-yl)-4-(naphthalen-2-yl- sulfonyl)butanamide |
| 26 | 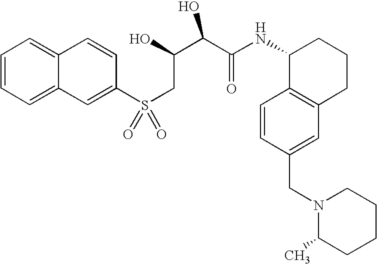 | (2R,3S)-2,3-dihydroxy-N-((R)-6-(((S)-2-meth- ylpiperidin-1-yl)methyl)-1,2,3,4-tetra- hydronaphthalen-1-yl)-4-(naphthalen-2-yl- sulfonyl)butanamide |
| 27 | 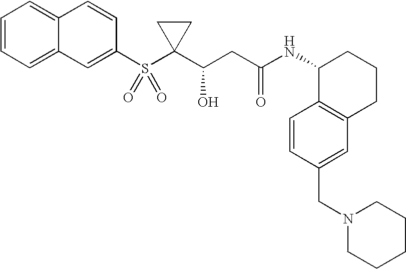 | (S)-3-hydroxy-3-(1-(naphthalen-2-yl- sulfonyl)cyclopropyl)-N-((R)-6-(piperidin-1-yl- methyl)-1,2,3,4-tetrahydronaphthalen-1-yl)pro- panamide |
| 28 | 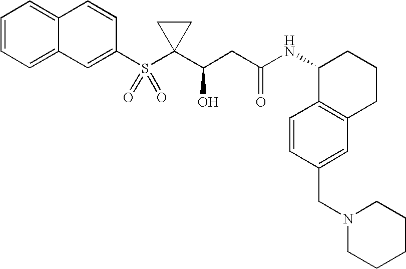 | (R)-3-hydroxy-3-(1-(naphthalen-2-yl- sulfonyl)cyclopropyl)-N-((R)-6-(piperidin-1-yl- methyl)-1,2,3,4-tetrahydronaphthalen-1-yl)pro- panamide |
| 29 | 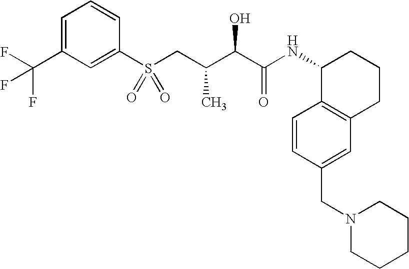 | (2R,3S)-2-hydroxy-3-methyl-N-((R)-6-(piperidin-1-yl- methyl)-1,2,3,4-tetrahydro-naphthalen-1-yl)-4-(3-(tri- fluoromethyl)-phenylsulfonyl)butanamide |
| 30 | 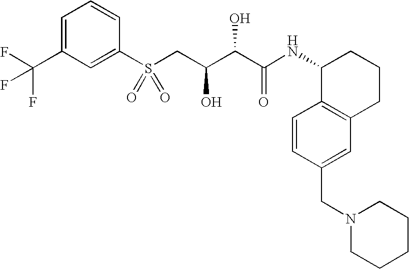 | (2S,3R)-2,3-dihydroxy-N-((R)-6-(piperidin-1-yl- methyl)-1,2,3,4-tetrahydronaphthalen-1-yl)-4-(3-(tri- fluoromethyl)phenyl-sulfonyl)butanamide |
| 31 | 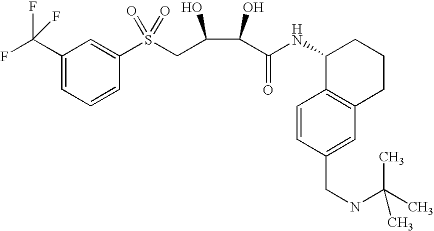 | (2R,3S)-N-((R)-6-((tert-butylamino)methyl)-1,2,3,4-tetra- hydronaphthalen-1-yl)-2,3-di- hydroxy-4-(3-(trifluoromethyl)-phenyl- sulfonyl)butanamide |
| 32 | 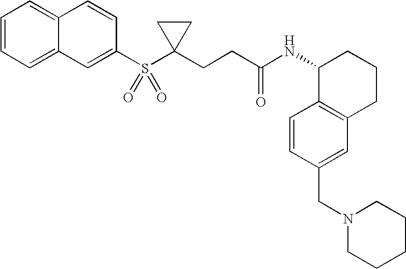 | (R)-3-(1-(naph- thalen-2-ylsulfonyl)-cyclopropyl)-N-(6-(pipe- ridin-1-ylmethyl)-1,2,3,4-tetra- hydronaphthalen-1-yl)propanamide |
| 33 | 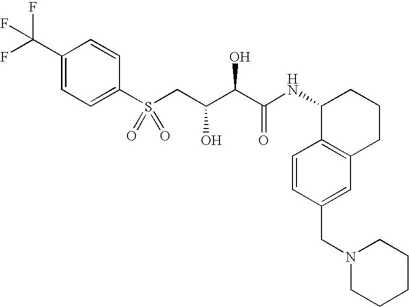 | (2R,3S)-2,3-dihydroxy-N-((R)-6-(piperidin-1-yl- methyl)-1,2,3,4-tetrahydronaphthalen-1-yl)-4-(4-(tri- fluoromethyl)phenyl-sulfonyl)butanamide |
| 34 | 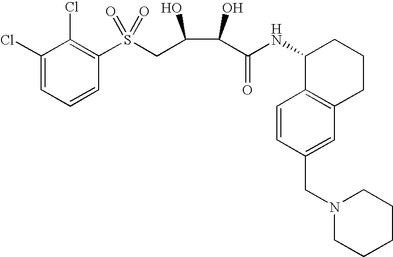 | (2R,3S)-4-(2,3-dichlorophenylsulfonyl)-2,3-di- hydroxy-N-((R)-6-(piperidin-1-ylmethyl)-1,2,3,4-tetra- hydronaphthalen-1-yl)-butanamide |
| 35 | 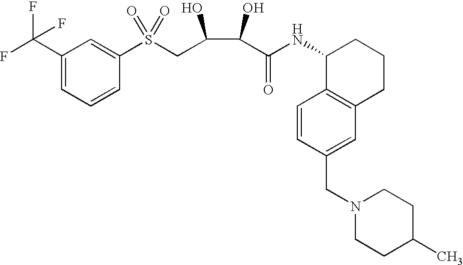 | (2R,3S)-2,3-dihydroxy-N-((R)-6-((4-methyl- piperidin-1-yl)methyl)-1,2,3,4-tetrahydro- naphthalen-1-yl)-4-(3-(trifluoromethyl)phenyl- sulfonyl)butanamide |
| 36 | 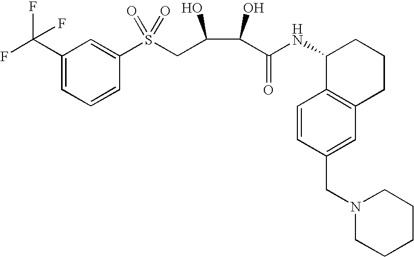 | (2R,3S)-2,3-dihydroxy-N-((R)-6-(piperidin-1-yl- methyl)-1,2,3,4-tetrahydronaphthalen-1-yl)-4-(3-(tri- fluoromethyl)phenyl-sulfonyl)butanamide |
| 37 | 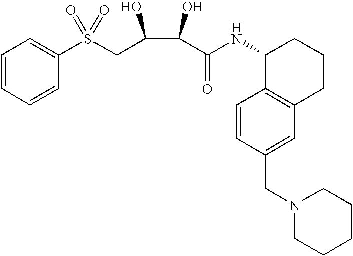 | (2R,3S)-2,3-di- hydroxy-4-(phenylsulfonyl)-N-((R)-6-(pipe- ridin-1-ylmethyl)-1,2,3,4-tetrahydro- naphthalen-1-yl)butanamide |
| 38 | 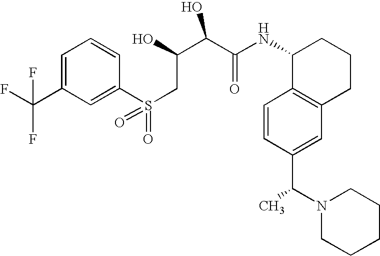 | (2R,3S)-2,3-dihydroxy-N-((R)-6-((R)-1-(pipe- ridin-1-yl)ethyl)-1,2,3,4-tetrahydro- naphthalen-1-yl)-4-(3-(trifluoromethyl)-phenyl- sulfonyl)butanamide |
| 39 | 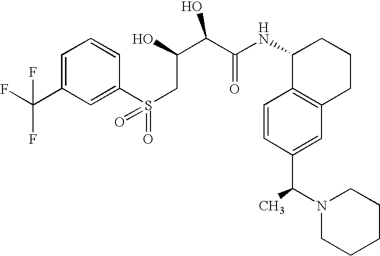 | (2R,3S)-2,3-dihydroxy-N-((R)-6-((S)-1-(pipe- ridin-1-yl)ethyl)-1,2,3,4-tetrahydro- naphthalen-1-yl)-4-(3-(trifluoromethyl)-phenyl- sulfonyl)butanamide |
| 40 | 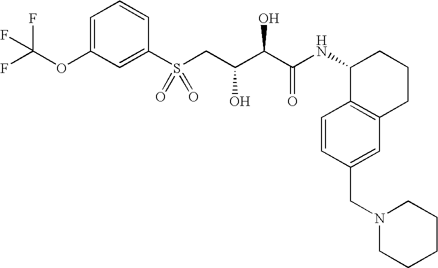 | (2R,3S)-2,3-dihydroxy-N-((R)-6-(piperidin-1-yl- methyl)-1,2,3,4-tetrahydronaphthalen-1-yl)-4-(3-(tri- fluoromethoxy)phenyl-sulfonyl)butanamide |
| 41 | 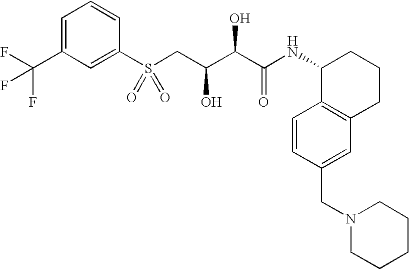 | (2R,3R)-2,3-dihydroxy-N-((R)-6-(piperidin-1-yl- methyl)-1,2,3,4-tetrahydronaphthalen-1-yl)-4-(3-(tri- fluoromethyl)phenyl-sulfonyl)butanamide |
| 42 | 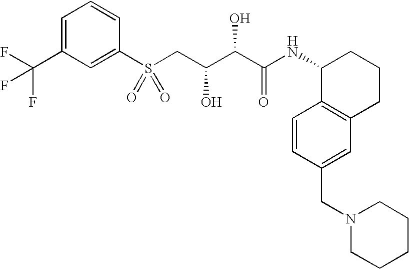 | (2S,3S)-2,3-dihydroxy-N-((R)-6-(piperidin-1-yl- methyl)-1,2,3,4-tetrahydronaphthalen-1-yl)-4-(3-(tri- fluoromethyl)phenylsulfonyl)-butanamide |
| 43 | 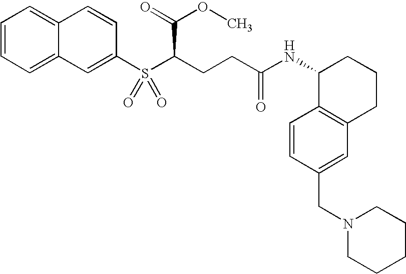 | (R)-methyl 2-(naph- thalen-2-ylsulfonyl)-5-oxo-5-((R)-6-(pipe- ridin-1-ylmethyl)-1,2,3,4-tetrahydro- naphthalen-1-ylamino)pentanoate |
| 44 | 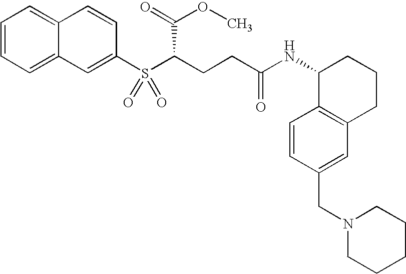 | (S)-methyl 2-(naph- thalen-2-ylsulfonyl)-5-oxo-5-((R)-6-(pipe- ridin-1-ylmethyl)-1,2,3,4-tetrahydro- naphthalen-1-ylamino)pentanoate |
| 45 | 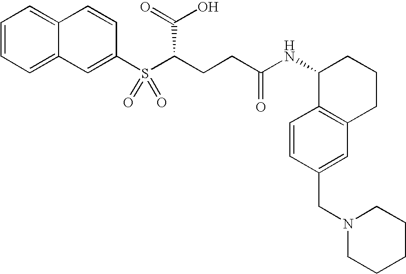 | (S)-2-(naphthalen-2-ylsulfonyl)-5-oxo-5-((R)-6-(pipe- ridin-1-ylmethyl)-1,2,3,4-tetrahydro- naphthalen-1-ylamino)pentanoic acid |
| 46 | 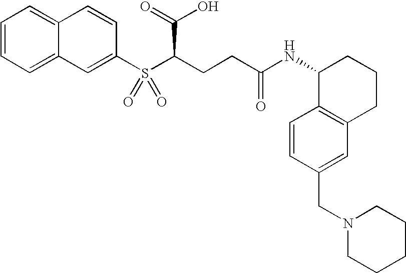 | (R)-2-(naphthalen-2-ylsulfonyl)-5-oxo-5-((R)-6-(pipe- ridin-1-ylmethyl)-1,2,3,4-tetrahydro- naphthalen-1-ylamino)pentanoic acid |
| 47 | 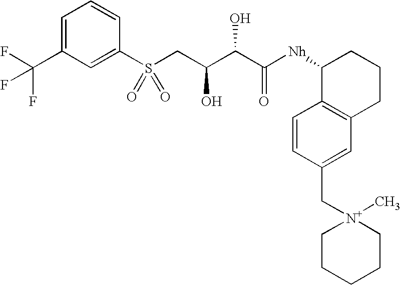 | ′1-(((5R)-5-(((2S,3R)-2,3-dihydroxy-4-((3-(tri- fluoromethyl)phenyl)sulfonyl)buta- noyl)amino)-5,6,7,8-tetra- hydro-2-naphthalenyl)methyl)-1-methyl- piperidinium |
| 48 | 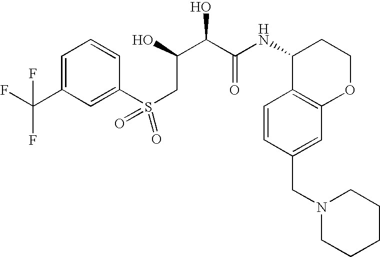 | (2R,3S)-2,3-dihydroxy-N-((R)-7-(piperidin-1-yl- methyl)chroman-4-yl)-4-(3-(trifluoromethyl)-phenyl- sulfonyl)-butanamide |
| 49 | 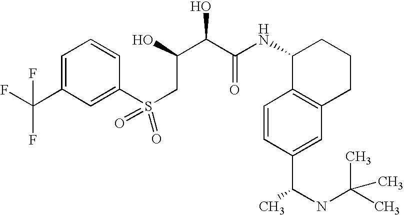 | (2R,3S)-N-((R)-6-((R)-1-(tert-butyl- amino)ethyl)-1,2,3,4-tetra- hydronaphthalen-1-yl)-2,3-di- hydroxy-4-(3-(trifluoromethyl)phenylsulfonyl)-butan- amide |
| 50 | 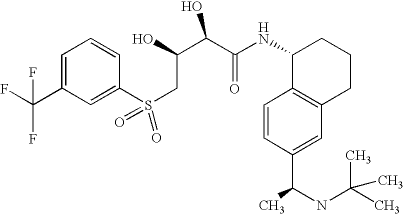 | (2R,3S)-N-((R)-6-((S)-1-(tert-butyl- amino)ethyl)-1,2,3,4-tetra- hydro-naphthalen-1-yl)-2,3-di- hydroxy-4-(3-(trifluoromethyl)phenylsulfonyl)-butan- amide |
| 51 | 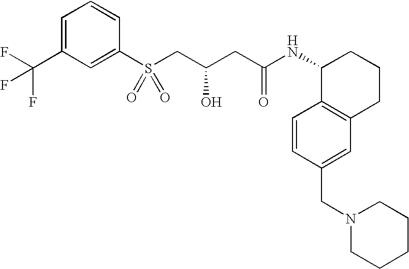 | (S)-3-hydroxy-N-((R)-6-(pipe- ridin-1-ylmethyl)-1,2,3,4-tetra- hydronaphthalen-1-yl)-4-(3-(tri- fluoromethyl)phenyl-sulfonyl)butanamide |
| 52 | 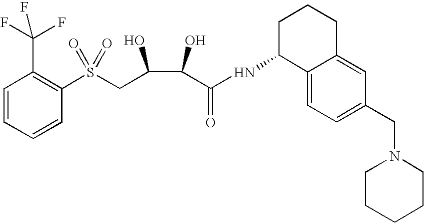 | (2R,3S)-2,3-dihydroxy-N-((R)-6-(piperidin-1-yl- methyl)-1,2,3,4-tetrahydronaphthalen-1-yl)-4-(2-(tri- fluoromethyl)phenylsulfonyl)-butanamide |
| 53 | 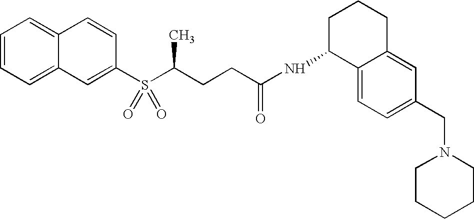 | (S)-4-(naphthalen-2-ylsulfonyl)-N-((R)-6-(pipe- ridin-1-ylmethyl)-1,2,3,4-tetrahydro- naphthalen-1-yl)pentanamide |
| 54 | 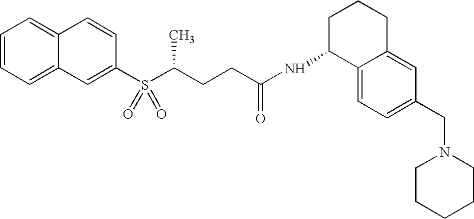 | (R)-4-(naphthalen-2-ylsulfonyl)-N-((R)-6-(pipe- ridin-1-ylmethyl)-1,2,3,4-tetrahydro- naphthalen-1-yl)pentanamide |
| 55 | 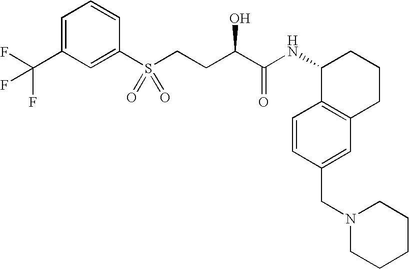 | (R)-2-hydroxy-N-((R)-6-(pipe- ridin-1-ylmethyl)-1,2,3,4-tetra- hydronaphthalen-1-yl)-4-(3-(tri- fluoromethyl)phenylsulfonyl)-butanamide |
| 56 | 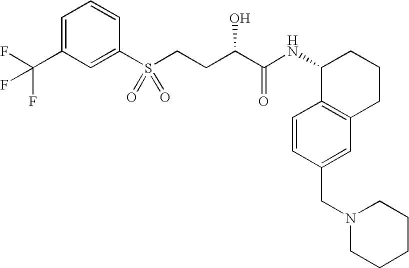 | (S)-2-hydroxy-N-((R)-6-(pipe- ridin-1-ylmethyl)-1,2,3,4-tetra- hydronaphthalen-1-yl)-4-(3-(tri- fluoromethyl)phenylsulfonyl)-butanamide |
| 57 | 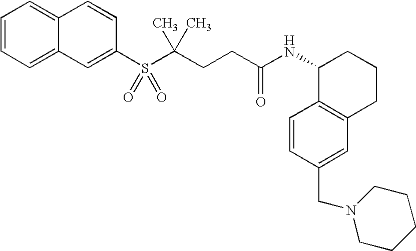 | (R)-4-methyl-4-(naphthalen-2-ylsulfonyl)-N-(6-(pipe- ridin-1-ylmethyl)-1,2,3,4-tetrahydro- naphthalen-1-yl)pentanamide |
| 58 | 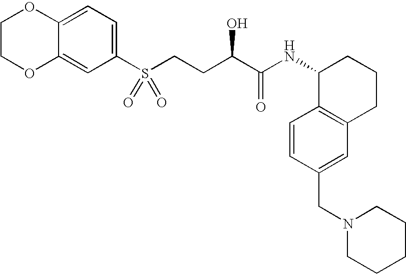 | (R)-4-(2,3-dihydrobenzo[b][1,4]dioxin-6-yl- sulfonyl)-2-hydroxy-N-((R)-6-(piperidin-1-yl- methyl)-1,2,3,4-tetrahydronaphthalen-1-yl)-butanamide |
| 59 | 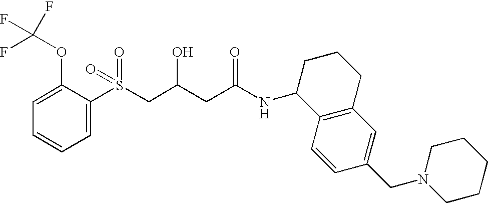 | 3-hydroxy-N-(6-(piperidin-1-ylmethyl)-1,2,3,4-tetra- hydronaphthalen-1-yl)-4-(2-(trifluoro- methoxy)phenylsulfonyl)butanamide |
| 60 | 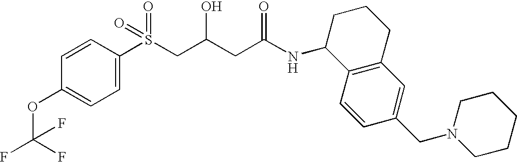 | 3-hydroxy-N-(6-(piperidin-1-ylmethyl)-1,2,3,4-tetra- hydronaphthalen-1-yl)-4-(4-(trifluoro- methoxy)phenylsulfonyl)butanamide |
| 61 | 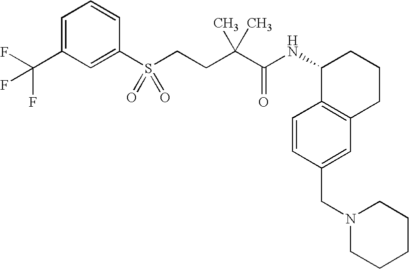 | (R)-2,2-dimeth- yl-N-(6-(piperidin-1-ylmethyl)-1,2,3,4-tetra- hydronaphthalen-1-yl)-4-(3-(tri- fluoromethyl)phenylsulfonyl)-butanamide |
| 62 | 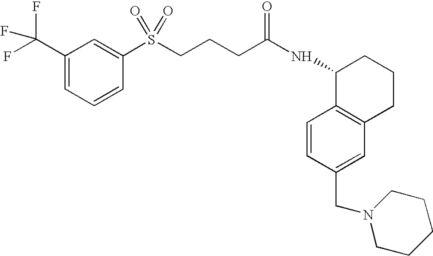 | (R)-N-(6-(piperidin-1-ylmethyl)-1,2,3,4-tetra- hydronaphthalen-1-yl)-4-(3-(trifluoro- methyl)phenylsulfonyl)butanamide |
| 63 | 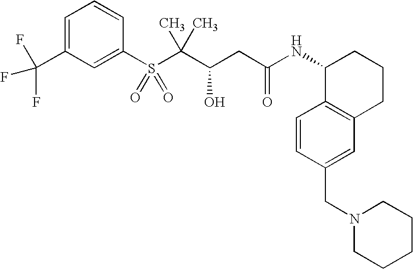 | (S)-3-hydroxy-4-methyl-N-((R)-6-(piperidin-1-yl- methyl)-1,2,3,4-tetrahydro-naphthalen-1-yl)-4-(3-(tri- fluoromethyl)-phenyl-sulfonyl)pentanamide |
| 64 | 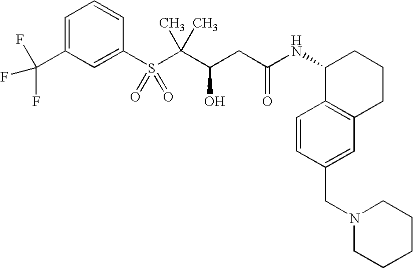 | (R)-3-hydroxy-4-methyl-N-((R)-6-(piperidin-1-yl- methyl)-1,2,3,4-tetrahydronaphthalen-1-yl)-4-(3-(tri- fluoro-methyl)phenylsulfonyl)pentanamide |

### Biological Testing

Although the pharmacological properties of the compounds of Formula I vary with structural change, in general, activity possessed by compounds of Formula I may be demonstrated in vivo. The pharmacological properties of the compounds of this invention may be confirmed by a number of pharmacological in vitro assays. The exemplified pharmacological assays, which follow, have been carried out with the compounds according to the invention and their salts.

Human Bradykinin B1 Receptor and human B2 Receptor In Vitro Binding Assays.

### Example 1

Radioligand Binding Assay for human B1 and human B2 bradykinin receptor.

Step 1 Preparation of membranes expressing human B1 bradykinin receptor:

Membranes were prepared from CHO-d′AQN cells stably transfected with human bradykinin B1 receptor cDNA. For large-scale production of membranes, cells were grown in 100 L suspension culture to 1.0E8 cells/mL then harvested using the Viafuge at continuous centrifugation of 1000 g. For pilot studies, cells were grown in 2 L spinner culture and harvested by centrifugation (1900 g, 10 min, 4° C.). The cell pellet was washed with PBS, centrifuged (1900 g, 10 min, 4° C.), then the cells resuspended in lysis buffer (25 mM HEPES, pH 7.4, 5 mM EDTA, 5 mM EGTA, 3 mM MgCl₂, 10% (w/v) sucrose, Complete Protease Inhibitor tablets (EDTA-free)) to a density of 14% w/v for passage through a microfluidizer (Microfluidics 110S, 3 passes, 6,000 psi). The resulting cell lysate was centrifuged (1900 g, 10 min, 4° C.), and the crude particulate fraction isolated by centrifugation (142,000 g, 1 h, 4° C.) of the low-speed supernatant. The resulting pellet was resuspended in ⅓ the original lysis buffer volume, homogenized, and recentrifuged as above. The membrane pellet was resuspended by homogenization in storage buffer (25 mM HEPES, pH 7.4, 3 mM MgCl₂, 10% (w/v) sucrose and Complete Protease Inhibitor tablets (EDTA-free)). Single-use aliquots were made and flash-frozen in liquid N₂ prior to storage at −80° C.

Membranes containing human bradykinin B2 receptor were purchased from Receptor Biology (now Perkin Elmer Life Sciences). They were derived from a CHO-K1 line stably expressing the human B2 receptor developed by Receptor Biology and subsequently purchased by Amgen. For some studies, membranes were prepared in-house from this same cell line using the method described for human B1 receptor membranes, except cells were grown in roller bottles and harvested using Cellmate.

Step 2 Human B1 receptor binding assay was performed in 96-well polypropylene plates (Costar 3365) by adding 50 μl [³H] des-arglo kallidin (NET1064; Perkin Elmer Life Sciences) to 10 μL test compound diluted in 90 μL assay buffer (24 mM TES, pH 6.8, 1 mM 1,10 o-phenanthroline, 0.3% BSA, 0.5 mM Pefabloc SC, 2 μg/mL aprotinin, 5 μg/mL leupeptin, and 0.7 μg/mL pepstatin A). Membranes (50 μL) were added last. [³H] des-arg¹⁰ kallidin was diluted from stock into assay buffer to yield a final concentration of 0.3 nM in the assay but was adjusted as needed to ensure a concentration at or below the K_{d} determined for each batch of receptor membranes. Nonspecific binding was defined with 2 μM des-Arg¹⁰Leu⁹ kallidin. Membranes were diluted in assay buffer to yield a final concentration of 0.068 nM hB1 receptor in the assay. Compounds were solubilized in either DMSO or ddH₂O, plated into polypropylene plates (Costar 3365), then serially diluted in either DMSO or dilution buffer (20 mM Hepes, pH 7.6, 0.1% BSA) to yield a final concentration of either 5% DMSO or no DMSO in the assay. The assay mixture was incubated with shaking for 1 h at RT and then filtered through GF/C plates presoaked in 0.5% polyethyleneimine (Unifilter; Perkin Elmer Life Sciences) using a Filtermate 96-well harvester (Perkin Elmer Life Sciences). Filter plates were rapidly washed 6 times with 200 μL ice-cold buffer (50 mM Tris, pH 7.4), dried in a vacuum oven at 55° C. for 15-20 min, backed, and 40 μL per well of Microscint 20 was added. The plates were sealed and activity read on Topcount (Perkin Elmer Life Sciences) using a count time of 3 min per channel.

For human B2 bradykinin receptor, the same procedure was followed with the following exceptions: [³H] bradykinin (NET706; Perkin Elmer Life Sciences) was used at a final concentration of 0.2 nM and non-specific binding was defined with 2 μM bradykinin. Human B2 receptor concentration was 0.068 nM final in the assay.

Data Analysis

Data was analyzed in XLFit with the four-parameter logistic y=A+((B−A)/(1+((C/x)ˆD))) and fit with the Levenburg-Marquardt algorithm. Raw cpm were converted to percent of control values prior to analysis (POC=((compound cpm−nonspecfic cpm)/(no-compound cpm−nonspecific cpm)*100)). Kᵢ values were determined from the IC₅₀ using the Cheng-Prusoff equation and K_{d} values determined by direct saturation binding of the radioligands.

### Example 2

### In Vitro B1-Inhibition Activity

In Vitro Assay of Human B1 Receptor Function Using Calcium Flux

Activation of the G_{q} linked B1 receptor results in an increase in intracellular calcium. The calcium sensitive photoprotein aequorin can, therefore, be used as an indicator of B1 receptor activation. Aequorin is a 21-kDa photoprotein that forms a bioluminescent complex when linked to the chromophore cofactor coelenterazine. Following the binding of calcium to this complex, an oxidation reaction of coelenterazine results in the production of apoacquorin, coelenteramide, CO₂, and light that can be detected by conventional luminometry.

A stable CHO D-/hB1/Aequorin cell line was established and the cells were maintained in suspension in spinner bottles containing a 1:1 ratio of DMEM and HAM F12 (Gibco 11765-047), high glucose (Gibco 11965-084), 10% Heat Inactivated Dialyzed serum (Gibco 26300-061), 1× Non-Essential Amino Acids (Gibco 11140-050), 1× Glutamine-Pen-Strep (Gibco 10378-016), and Hygromycin, 300 μg/mL (Roche 843555). 15-24 h prior to the luminometer assay, 25,000 cells/well (2.5E6 cells/10 mL/plate) were plated in 96-well black-sided clear bottom assay plates (Costar #3904).

Media was removed from the wells and replaced with 60 μL of serum free HAM's F12 with 30 mM HEPES (pH 7.5) and 15 μM coelenterazine (Coelenterazine h Luciferin #90608 from Assay Designs). The plates were incubated for 1.5-2 h. Ten point IC₅₀ compound plates containing 1:3 or 1:5 dilutions of antagonist compounds and an agonist activator plate (20 nM des-Arg10-Kallidin final concentration, EC₈₀) were prepared using Ham's F12 with 30 mM HEPES, pH 7.5. Following coelenterazine incubation, an automated flash-luminometer platform was used to dispense the B1 antagonist compounds (dissolved in DMSO and diluted with buffer to the desired concentration (final DMSO concentration <1% DMSO)) to the cell plate, a CCD camera situated underneath the cell plate took 12 images of the cell plate at 5 second intervals to determine if there was any agonist activity with the compounds. The hB1 agonist, des-Arg₁₀-Kallidin, was added to the cell plate and another 12 images were recorded to determine the IC₅₀ of the antagonist(s).

In Vitro Assay of hB2 Receptor Function Using Calcium Flux

The intracellular calcium flux induced by hB2 receptor activation was analyzed using an hB2 recombinant cell line (CHO-K1) purchased from PerkinElmer (Catalog Number: RBHB2C000EA) on a fluorometric imaging plate reader (FLIPR). The cells were cultured in T225 flask containing Ham's F12 Nutrient Mixture (Invitrogen Corp., Cat # 11765-047), 10% Fetal Clone II Bovine Serum (HyClone, Cat # SH3006603), 1 mM Sodium pyruvate (100 mM stock, Invitrogen Corp., Cat# 12454-013), and 0.4 mg/mL Geneticin (G418; 50 mg/mL active geneticin, Invitrogen, Cat# 10131-207). Culture medium was changed every other day. 24 h prior to the FLIPR assay, the hB2/CHO cells were washed once with PBS (Invitrogen) and 10 mL of Versene (1:5000, Invitrogen, Cat# 15040-066) was added to each flask. After 5 min incubation at 37° C., Versene was removed and cells were detached from the flask and resuspended in culture medium. Cells were counted and 25,000 cells/well were plated in 96-well black-sided clear bottom assay plates (Costar #3904). Cells were incubated in a 37° C. CO₂ incubator overnight.

The media was aspirated from the cells and replaced with 65 μL of dye-loading buffer. The loading buffer was prepared by diluting a stock solution of 0.5 mM Fluo-4 AM (Molecular Probes, dissolved in DMSO containing 10% [w/v] pluronic acid) to a concentration of 1 μM in Clear Dulbecco's Modified Eagle Medium (DMEM) containing 0.1% BSA, 20 mM HEPES, and 2.5 mM probenecid. The cells were dye-loaded for 1 h at RT. The excess dye was removed by washing the cells 2× with assay buffer. The assay buffer consists of Hank's Balanced Salt Solution (HBSS) containing 20 mM HEPES, 0.1% BSA, and 2.5 mM probenecid. After the wash cycles, a volume of 100 μL was left in each well, and the plate was ready to be assayed in the FLIPR System. Single point (10 μM final concentration) POC antagonist compound plates or ten point IC₅₀ compound plates containing 1:3 or 1:5 dilutions of antagonist compounds (dissolved in DMSO and diluted with buffer to the desired concentration (final DMSO concentration <1% DMSO)) and an agonist activator plate (0.3 nM bradykinin final concentration, EC₈₀) were prepared using assay buffer. The cell plate and the compound plates were loaded onto the FLIPR and during the assay, fluorescence readings are taken simultaneously from all 96 wells of the cell plate. Ten 1-second readings were taken to establish a stable baseline for each well, then 25 μL from the B1 antagonist plate was rapidly (50 μL/sec.) added. The fluorescence signal was measured in 1-second (1 min) followed by 6-second (2 min) intervals for a total of 3 min to determine if there is any agonist activity with the compounds. The B2 agonist, bradykinin, was added to the cell plate and another 3 min were recorded to determine the percent inhibition at 10 μM (POC plates) or the IC₅₀ of the antagonist.

### Example 3

Cell and Tissue Based In Vitro Assays of hB1 Receptor Binding

These studies established the antagonist activity of several compounds at the bradykinin B1 receptors in in vitro cell-based and isolated organ assays.

Rabbit endothelial cell B1-specific PGI₂ secretion Assay.

B1 and B2 umbilical vein Assay.

In Vitro B1-Inhibition Activity:

The effectiveness of the compounds as inhibitors of B1 activity (i.e., B1 “neutralization”) can be evaluated by measuring the ability of each compound to block B1 stimulated CGRP and substance P release and calcium signaling in Dorsal Root Ganglion (DRG) neuronal cultures.

Dorsal Root Ganglion Neuronal Cultures:

Dorsal root ganglia are dissected one by one under aseptic conditions from all spinal segments of embryonic 19-day old (E19) rats that are surgically removed from the uterus of timed-pregnant, terminally anesthetized Sprague-Dawley rats (Charles River, Wilmington, Mass.). DRG are collected in ice-cold L-15 media (GibcoBRL, Grand Island, N.Y.) containing 5% heat inactivated horse serum (GibcoBRL), and any loose connective tissue and blood vessels are removed. The DRG are rinsed twice in Ca²⁺- and Mg²⁺-free Dulbecco's phosphate buffered saline (DPBS), pH 7.4 (GibcoBRL). The DRG are dissociated into single cell suspension using a papain dissociation system (Worthington Biochemical Corp., Freehold, N.J.). Briefly, DRG are incubated in a digestion solution containing 20 U/mL of papain in Earle's Balanced Salt Solution (EBSS) at 37° C. for fifty minutes. Cells are dissociated by trituration through fire-polished Pasteur pipettes in a dissociation medium consisting of MEM/Ham's F12, 1:1, 1 mg/mL ovomucoid inhibitor and 1 mg/mL ovalbumin, and 0.005% deoxyribonuclease I (DNase). The dissociated cells are pelleted at 200×g for 5 min and re-suspended in EBSS containing 1 mg/mL ovomucoid inhibitor, 1 mg/mL ovalbumin and 0.005% DNase. Cell suspension is centrifuged through a gradient solution containing 10 mg/mL ovomucoid inhibitor, 10 mg/mL ovalbumin at 200×g for 6 min to remove cell debris, then filtered through a 88-EM nylon mesh (Fisher Scientific, Pittsburgh, Pa.) to remove any clumps. Cell number is determined with a hemocytometer, and cells are seeded into poly-ornithine 100 μg/mL (Sigma, St. Louis, Mo.) and mouse laminin 1 μg/mL (GibcoBRL)-coated 96-well plates at 10×10³ cells/well in complete medium. The complete medium consists of minimal essential medium (MEM) and Ham's F12, 1:1, penicillin (100 U/mL), streptomycin (100 μg/mL), and 10% heat inactivated horse serum (GibcoBRL). The cultures are kept at 37° C., 5% CO₂ and 100% humidity. For controlling the growth of non-neuronal cells, 5-fluoro-2′-deoxyuridine (75 μM) and uridine (180 μM) are included in the medium.

Two hours after plating, cells are treated with recombinant human β-b1 or recombinant rat β-b1 at a concentration of 10 mg/ml (0.38 nm). Positive controls comprising serial-diluted anti-b1 antibody (r&d systems, Minneapolis, Minn.) are applied to each culture plate. Compounds are added at ten concentrations using 3.16-fold serial dilutions. All samples are diluted in complete medium before being added to the cultures. Incubation time is generally around 40 h prior to measurement of vr1 expression.

Measurement of VR1 Expression in DRG Neurons:

Cultures are fixed with 4% paraformaldehyde in Hanks' balanced salt solution for 15 min, blocked with Superblock (Pierce, Rockford, Ill.), and permeabilized with 0.25% Nonidet P-40 (Sigma) in Tris.HCl (Sigma)-buffered saline (TBS) for 1 h at RT. Cultures are rinsed once with TBS containing 0.1% Tween 20 (Sigma) and incubated with rabbit anti-VR1 IgG (prepared at Amgen) for 1.5 h at RT, followed by incubation of Eu-labeled anti-rabbit second antibody (Wallac Oy, Turku, Finland) for 1 h at RT. Washes with TBS (3× five min with slow shaking) are applied after each antibody incubation. Enhance solution (150 mL/well, Wallac Oy) is added to the cultures. The fluorescence signal is measured in a time-resolved fluorometer (Wallac Oy). VR1 expression in samples treated with the compounds is determined by comparing to a standard curve of B1 titration from 0-1000 ng/mL. Percent inhibition (compared to maximum possible inhibition) of B1 effect on VR1 expression in DRG neurons is determined by comparing to controls that are not B1-treated.

### Example 4

In Vivo Antinociceptive Activity in Rat and Monkey Pain Models

Rat Neuropathic Pain Model

Male Sprague-Dawley rats (200 g) are anesthetized with isoflurane inhalant anesthesia and the left lumbar spinal nerves at the level of L5 and L6 are tightly ligated (4-0 silk suture) distal to the dorsal root ganglion and prior to entrance into the sciatic nerve, as first described by Kim and Chung (Kim, S. H.; Chung, J. M. An experimental model for peripheral neuropathy produced by segmental spinal nerve ligation in the rat. Pain 50:355-363, (1992)). The incisions are closed and the rats are allowed to recover. This procedure results in mechanical (tactile) allodynia in the left hind paw as assessed by recording the pressure at which the affected paw (ipsilateral to the site of nerve injury) was withdrawn from graded stimuli (von Frey filaments ranging from 4.0 to 148.1 mN) applied perpendicularly to the plantar surface of the paw (between the footpads) through wire-mesh observation cages. A paw withdrawal threshold (PWT) was determined by sequentially increasing and decreasing the stimulus strength and analyzing withdrawal data using a Dixon non-parametric test, as described by Chaplan et al. (Chaplan, S. R.; Bach, F. W.; Pogrel, J. W.; Chung, J. M.; Yaksh, T. L. Quantitative assessment of tactile allodynia in the rat paw. J. Neurosci. Meth., 53:55-63 (1994)).

Normal rats and sham surgery rats (nerves isolated but not ligated) withstand at least 148.1 mN (equivalent to 15 g) of pressure without responding. Spinal nerve ligated rats respond to as little as 4.0 mN (equivalent to 0.41 g) of pressure on the affected paw. Rats are included in the study only if they did not exhibit motor dysfunction (e.g., paw dragging or dropping) and their PWT was below 39.2 mN (equivalent to 4.0 g). At least seven days after surgery rats are treated with compounds (usually a screening dose of 60 mg/kg) or control diluent (PBS) once by s.c. injection and PWT was determined each day thereafter for 7 days.

Rat CFA Inflammatory Pain Model

Male Sprague-Dawley rats (200 g) are lightly anesthetized with isoflurane inhalant anesthesia and the left hindpaw is injected with complete Freund's adjuvant (CFA), 0.15 mL. This procedure results in mechanical (tactile) allodynia in the left hind paw as assessed by recording the pressure at which the affected paw is withdrawn from graded stimuli (von Frey filaments ranging from 4.0 to 148.1 mN) applied perpendicularly to the plantar surface of the paw (between the footpads) through wire-mesh observation cages. PWT is determined by sequentially increasing and decreasing the stimulus strength and analyzing withdrawal data using a Dixon non-parametric test, as described by Chaplan et al. (1994). Rats are included in the study only if they do not exhibit motor dysfunction (e.g., paw dragging or dropping) or broken skin and their PWT is below 39.2 mN (equivalent to 4.0 g). At least seven days after CFA injection rats are treated with compounds (usually a screening dose of 60 mg/kg) or control solution (PBS) once by s.c. injection and PWT is determined each day thereafter for 7 days. Average paw withdrawal threshold (PWT) is converted to percent of maximum possible effect (% MPE) using the following formula: % MPE=100*(PWT of treated rats−PWT of control rats)/(15−PWT of control rats). Thus, the cutoff value of 15 g (148.1 mN) is equivalent to 100% of the MPE and the control response is equivalent to 0% MPE.

At the screening dose of 60 mg/kg, compounds in vehicle are expected to produce an antinociceptive effect with a PD relationship.

### Example 5

Green Monkey LPS Inflammation Model

The effectiveness of the compounds as inhibitors of B1 activity are evaluated in Male green monkeys (Cercopithaecus aethiops St Kitts) challenged locally with B1 agonists essentially as described by deBlois and Horlick (British Journal of Pharmacology, 132:327-335 (2002), which is hereby incorporated by reference in its entirety).

In order to determine whether compounds of the present invention inhibit B1 induced oedema the studies described below are conducted on male green monkeys (Cercopithaecus aethiops St Kitts) at the Caribbean Primates Ltd. experimental farm (St Kitts, West Indies). Procedures are reviewed and accepted by the Animal Care Committees of the CR-CHUM (Montreal, Canada) and of Caribbean Primates Ltd. (St Kitts, West Indies). Animals weighing 6.0±0.5 kg (n=67) were anaesthetized (50 mg ketamine kg⁻¹) and pretreated with a single intravenous injection of LPS (90 μg kg⁻¹) or saline (1 mL) via the saphenous vein.

Inflammation Studies

Kinin-induced oedema is evaluated by the ventral skin fold assay (Sciberras et al., 1987). Briefly, anaesthetized monkeys were injected with captopril (1 mg kg⁻¹ 30 min before assay). A single subcutaneous injection of dKD, BK or the vehicle (2 mM amastatin in 100 μL Ringer's lactate) is given in the ventral area and the increase in thickness of skin folds is monitored for 30-45 min using a calibrated caliper. The results are expressed as the difference between the skin fold thickness before and after the subcutaneous injection. Captopril and amastatin are used to reduce degradation of kinins at the carboxyl- and amino-terminus, respectively.

Antagonist Schild Analysis:

The dose-response relationship for dKD (1-100 nmol)-induced oedema is determined at 24 h post-LPS in the absence or presence of different concentrations of antagonist. BK (30 nmol) is used as a positive control.

Antagonist Time Course

The time course of inhibition by antagonist is determined at 4, 24 and 48 h, 72 and/or 96 h after single bolus administration. BK (30 nmol) is used as a positive control.

Drugs

Ketamine hydrochloride, LPS, amastatin and captopril are from Sigma (MO, U.S.A.). All peptides are from Phoenix Pharmaceuticals (CA, U.S.A.).

Statistics

Values are presented as mean±standard error of the mean (s.e. mean). In edema studies, the pre-injection thickness of the skin folds was subtracted from the values after subcutaneous challenge. Curve fitting and EC₅₀ calculations were obtained using the Delta Graph 4.0 software for Apple Computers. Data were compared by two-way analysis of variance followed by unpaired, one tail Student's t-test with Bonferroni correction. P<0.05 was considered statistically significant.

The foregoing is merely illustrative of the invention and is not intended to limit the invention to the disclosed compounds. Variations and changes which are obvious to one skilled in the art are intended to be within the scope and nature of the invention which are defined in the appended claims.

From the foregoing description, one skilled in the art can easily ascertain the essential characteristics of this invention, and without departing from the spirit and scope thereof, can make various changes and modifications of the invention to adapt it to various usages and conditions.

No unacceptable toxological effects are expected when compounds of the present invention are administered in accordance with the present invention.

All mentioned references, patents, applications and publications, are hereby incorporated by reference in their entirety, as if here written.

What is claimed is:

1. A compound of Formula (I): 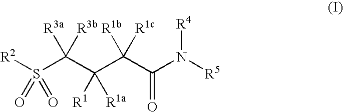
wherein:
R¹ is selected from H, R^{g}, halo, cyano, nitro, —C(═O)R^{b}, —C(═O)OR^{a}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylOR^{a}, benzyl and C₁₋₆alkyl, with the benzyl and C₁₋₆alkyl being substituted by 0, 1, 2, or 3 groups independently selected from R^{g}, cyano, oxo, nitro, —C(═O)R^{b}, —C(═O)OR^{a}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆ alkylNR^{a}R^{a} and —NR^{a}C₂₋₆alkylOR^{a}, and additionally substituted by 0, 1, 2, 3, 4, 5 or 6 atoms selected from Br, Cl, F and I;
R^{1a} and R^{1b} are each independently, H, F, Cl, —OH, OCH₃, C₁₋₂alkyl or CF₃;
R^{1c} is H, C₁₋₈alkyl, C₁₋₄haloalkyl, halo, cyano, nitro, —C(═O)R^{b}, —C(═O)OR^{b}, C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}, or C₁₋₆alkyl substituted by 0, 1, 2 or 3 substituents independently selected from C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{b}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} or —NR^{a}C₂₋₆alkylOR^{a};
R^{3a} is H, R^{g}, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}, benzyl or C₁₋₆alkyl, with the benzyl and C₁₋₆alkyl being substituted by 0, 1, 2 or 3 substituents independently selected from R^{e}, R^{g}, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1, 2, 3, 4, 5 or 6 atom selected from Br, Cl, F and I;
R^{3b} is H, F, Cl, OCH₃, C₁₋₂alkyl or CF₃; or
R^{3a} and R^{3b} together are C₂₋₆alkylenyl to form a spiroalkyl that is substituted by 0, 1, 2 or 3 substituents independently selected from R^{e}, R^{g}, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆ alkylNR^{a}R^{a} and —NR^{a}C₂₋₆alkylOR^{a}, and additionally substituted by 0, 1, 2, 3, 4, 5 or 6 atom selected from Br, Cl, F and I;
R² is a saturated, partially saturated or unsaturated 5-, 6- or 7-membered monocyclic or 6-, 7-, 8-, 9-, 10- or 11-membered bicyclic hydrocarbon ring containing 0, 1, 2, 3 or 4 atoms independently selected from N, O and S, wherein the carbon atoms of the ring are substituted by 0, 1 or 2 oxo groups and the ring is substituted by 0, 1, 2 or 3 substituents selected from R^{e}, R^{g}, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and the ring is additionally substituted by 0, 1, 2, 3, 4 or 5 substituents independently selected from Br, Cl, F and I;
R⁴ is H, phenyl, benzyl or C₁₋₆alkyl, the phenyl, benzyl and C₁₋₆alkyl being substituted by 0, 1, 2 or 3 substituents independently selected from C₁₋₄alkyl, C₁₋₃haloalkyl, —OC₁₋₄alkyl, —NH₂, —NHC₁₋₄alkyl, and —N(C₁₋₄alkyl)C₁₋₄alkyl, and additionally substituted by 0, 1, 2, 3, 4, 5 or 6 atom selected from Br, Cl, F and I;
R⁵ is -(alkylene)n-R where n is 0 or 1 and R is a 5-, 6-, 7-, or 8-membered saturated, partially saturated or unsaturated monocyclic, a saturated, partially saturated or unsaturated 8-, 9-, 10- or 11-membered bicyclic or 12-, 13-, 14- or 15-membered tricyclic hydrocarbon ring containing 0, 1, 2, 3 or 4 atoms selected from N, O and S, wherein the carbon and sulfur atoms of the ring are substituted by 0, 1 or 2 oxo groups and the ring is substituted by R⁶, R⁷ or R⁸ independently selected from basic moieties, and additionally substituted by 0, 1, 2 or 3 substituents selected from R⁶, R⁷ and R⁸ which are selected from R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and the ring is additionally substituted by 0, 1, 2, 3, 4 or 5 substituents selected from R⁶, R⁷, R⁸, R⁹ and R¹⁰ which are independently selected from Br, Cl, F and I; or
R^{1c} and R⁴ together may additionally be C₂₋₄alkylene substituted by 0, 1 or 2 substituents independently selected from C₁₋₈alkyl, C₁₋₄haloalkyl, halo, oxo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1, 2, 3 or 4 substituents independently selected from Br, Cl, F and I;
R^{a} is independently, at each instance, H or R^{b};
R^{b} is independently, at each instance, phenyl, benzyl or C₁₋₆alkyl, the phenyl, benzyl and C₁₋₆alkyl being substituted by 0, 1, 2 or 3 substituents selected from halo, C₁₋₄alkyl, C₁₋₃haloalkyl, —OC₁₋₄alkyl, —NH₂, —NHC₁₋₄alkyl, —N(C₁₋₄alkyl)C₁₋₄alkyl;
R^{d} is independently, at each instance, C₁₋₈alkyl, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} or NR^{a}C₂₋₆alkylOR^{a};
R^{e} is independently, at each instance, C₁₋₆alkyl substituted by 0, 1, 2 or 3 substituents independently selected from R^{d} and additionally substituted by 0 or 1 substituents selected from R^{g}; and
R^{g} is independently, at each instance, a saturated, partially saturated or unsaturated 5-, 6- or 7-membered monocyclic or 6-, 7-, 8-, 9-, 10- or 11-membered bicyclic hydrocarbon ring containing 0, 1, 2, 3 or 4 atoms selected from N, O and S, wherein the carbon atoms of the ring are substituted by 0, 1 or 2 oxo groups and the ring is substituted by 0, 1, 2 or 3 substituents selected from C₁₋₈alkyl, C₁₋₄haloalkyl, halo, cyano, nitro, —C(═O)R^{b}, —C(═O)OR^{b}, C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R a)C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆ alkylNR^{a}R^{a} and —NR^{a}C₂₋₆alkylOR^{a}, and the ring is additionally substituted by 0, 1, 2, 3, 4 or 5 substituents independently selected from Br, Cl, F and I; or
any pharmaceutically-acceptable salt or hydrate thereof.

2. The compound of claim 1 wherein:
R¹ is selected from H, R^{g}, cyano, nitro, —C(═O)R^{b}, —C(═O)OR^{a}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylOR^{a}, benzyl and C₁₋₆alkyl, with the benzyl and C₁₋₆alkyl being substituted by 0, 1, 2, or 3 groups independently selected from R^{g}, cyano, oxo, nitro, —C(═O)R^{b}, —C(═O)OR^{a}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1, 2, 3, 4, 5 or 6 atoms selected from Br, Cl, F and I;
R⁵ is a saturated, partially saturated or unsaturated 8-, 9-, 10- or 11-membered bicyclic or 12-, 13-, 14- or 15-membered tricyclic ring hydrocarbon containing 0, 1, 2, 3 or 4 atoms selected from N, O and S, wherein the carbon and sulfur atoms of the ring are substituted by 0, 1 or 2 oxo groups and the ring is substituted by R⁶, R⁷ or R⁸ independently selected from basic moieties, and additionally substituted by 0, 1, 2 or 3 substituents independently selected from R⁶, R⁷ and R⁸ which are independently selected from R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and the ring is additionally substituted by 0, 1, 2, 3, 4 or 5 substituents selected from R⁶, R⁷, R⁸, R⁹ and R¹⁰ which are independently selected from Br, Cl, F and I; and
R^{1a} and R^{1b} are each independently, H, F, Cl, OCH₃, C₁₋₂alkyl or CF₃.

3. The compound of claim 2 wherein the basic moiety is amino, cycloalkylamino-(C₁-C₆)alkyl, cycloalkyl(C₁-C₆)alkylamino(C₁-C₆)alkyl, heterocyclylamino(C₁-C₆)alkyl, heterocyclyl(C₁-C₆)alkylamino(C₁-C₆)alkyl, arylamino(C₁-C₆)alkyl, aryl(C₁-C₆)alkylamino-(C₁-C₆)alkyl, (C₁-C₆)alkylamino(C₁-C₆)alkoxy, (C₁-C₆)alkylamino(C₁-C₆)alkoxy(C₁-C₆)-alkoxy, amino(C₁-C₆)alkoxy, amino(C₁-C₆)alkyl, (C₁-C₆)alkylamino(C₁-C₆)alkyl, (C₁-C₄)alkylamino-(C₂-C₆)alkenyl, 4-8-membered nitrogen-containing heterocyclyl(C₂-C₆)alkenyl, 5-6 membered heterocyclyloxy, 5-6 membered nitrogen-containing heterocyclyl and 5-7 membered nitrogen-containing heterocyclylalkyl; more specifically amino, cycloalkylamino(C₁-C₆)alkyl, cycloalkyl(C₁-C₆)alkylamino-(C₁-C₆)alkyl, heterocyclylamino(C₁-C₆)alkyl, heterocyclyl(C₁-C₆)alkylamino-(C₁-C₆)alkyl, arylamino(C₁-C₆)alkyl, aryl(C₁-C₆)alkylamino(C₁-C₆)alkyl, (C₁-C₆)alkyl amino(C₁-C₆)alkoxy, (C₁-C₆)alkylamino(C₁-C₆)alkoxy(C₁-C₆)alkoxy, amino(C₁-C₆)alkoxy, amino(C₁-C₆)alkyl, (C₁-C₆)alkylamino(C₁-C₆)alkyl, (C₁-C₄)alkylamino-(C₂-C₆)alkenyl, 5-8-membered nitrogen-containing heterocyclyl(C₂-C₆)alkenyl, heterocyclyl(C₁-C₆)amino(C₂-C₆)alkyl, 5-6 membered heterocyclyloxy, 5-6 membered nitrogen-containing heterocyclyl and 5-7 membered nitrogen-containing heterocyclyl(C₁-C₆)alkyl wherein the basic moiety can be substituted by 0, 1, 2 or 3 groups independently selected from halo, —NH₂, —OH, —CN, —CF₃, (C₁-C₆)alkylamino, haloalkyl, oxo, (C₁-C₆)alkoxy, (C₁-C₆)alkoxyalkyl, (C₂-C₆)alkenyl, (C₂-C₆)alkynyl, di(C₁-C₆)alkylamino, ═NCN; and (C₁-C₆)alkyl, aryl, heteroaryl, cycloalkyl and heterocyclyl, each of which is substituted by 0, 1, 2 or 3 groups independently selected from halo, —NH₂, —OH, —CN, —CF₃, (C₁-C₆)alkylamino, haloalkyl, oxo, (C₁-C₆)alkoxy, (C₁-C₆)alkoxyalkyl, (C₁-C₆)alkyl, (C₂-C₆)alkenyl, (C₂-C₆)alkynyl, or di(C₁-C₆)alkylamino.

4. The compound of claim 1 wherein R⁵ is -(alkylene)n-R where n is 0 or 1 and R is a 5-, 6-, 7-, or 8-membered saturated, partially saturated or unsaturated monocyclic hydrocarbon ring containing 0, 1, 2, 3 or 4 atoms selected from N, O and S, wherein the carbon and sulfur atoms of the ring are substituted by 0, 1 or 2 oxo groups and the ring is substituted by 1, 2 or 3 substituents selected from R⁶, R⁷ or R⁸ independently selected from basic moieties, and additionally substituted by 0, 1, 2 or 3 substituents selected from R⁶, R⁷ and R⁸ which are selected from R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and the ring is additionally substituted by 0, 1, 2, 3, 4 or 5 substituents selected from R⁶, R⁷, R⁸, R⁹ and R¹⁰ which are independently selected from Br, Cl, F and I.

5. The compound of claim 4 wherein the basic moiety is amino, cycloalkylamino-(C₁-C₆)alkyl, cycloalkyl(C₁-C₆)alkylamino(C₁-C₆)alkyl, heterocyclylamino(C₁-C₆)alkyl, heterocyclyl(C₁-C₆)alkylamino(C₁-C₆)alkyl, arylamino(C₁-C₆)alkyl, aryl(C₁-C₆)alkylamino-(C₁-C₆)alkyl, (C₁-C₆)alkylamino(C₁-C₆)alkoxy, (C₁-C₆)alkylamino(C₁-C₆)alkoxy(C₁-C₆)-alkoxy, amino(C₁-C₆)alkoxy, amino(C₁-C₆)alkyl, (C₁-C₆)alkylamino(C₁-C₆)alkyl, (C₁-C₄)alkylamino-(C₂-C₆)alkenyl, 4-8-membered nitrogen-containing heterocyclyl(C₂-C₆)alkenyl, 5-6 membered heterocyclyloxy, 5-6 membered nitrogen-containing heterocyclyl and 5-7 membered nitrogen-containing heterocyclylalkyl; more specifically amino, cycloalkylamino(C₁-C₆)alkyl, cycloalkyl(C₁-C₆)alkylamino-(C₁-C₆)alkyl, heterocyclylamino(C₁-C₆)alkyl, heterocyclyl(C₁-C₆)alkylamino-(C₁-C₆)alkyl, arylamino(C₁-C₆)alkyl, aryl(C₁-C₆)alkylamino(C₁-C₆)alkyl, (C₁-C₆)alkyl amino(C₁-C₆)alkoxy, (C₁-C₆)alkylamino(C₁-C₆)alkoxy(C₁-C₆)alkoxy, amino(C₁-C₆)alkoxy, amino(C₁-C₆)alkyl, (C₁-C₆)alkylamino(C₁-C₆)alkyl, (C₁-C₄)alkylamino-(C₂-C₆)alkenyl, 5-8-membered nitrogen-containing heterocyclyl(C₂-C₆)alkenyl, heterocyclyl(C₁-C₆)amino(C₂-C₆)alkyl, 5-6 membered heterocyclyloxy, 5-6 membered nitrogen-containing heterocyclyl and 5-7 membered nitrogen-containing heterocyclyl(C₁-C₆)alkyl wherein the basic moiety can be substituted by 0, 1, 2 or 3 groups independently selected from halo, —NH₂, —OH, —CN, —CF₃, (C₁-C₆)alkylamino, haloalkyl, oxo, (C₁-C₆)alkoxy, (C₁-C₆)alkoxyalkyl, (C₂-C₆)alkenyl, (C₂-C₆)alkynyl, di(C₁-C₆)alkylamino, ═NCN; and (C₁-C₆)alkyl, aryl, heteroaryl, cycloalkyl and heterocyclyl, each of which is substituted by 0, 1, 2 or 3 groups independently selected from halo, —NH₂, —OH, —CN, —CF₃, (C₁-C₆)alkylamino, haloalkyl, oxo, (C₁-C₆)alkoxy, (C₁-C₆)alkoxyalkyl, (C₁-C₆)alkyl, (C₂-C₆)alkenyl, (C₂-C₆)alkynyl, or di(C₁-C₆)alkylamino.

6. The compound of claim 3 wherein:
R^{3a} is hydrogen, C₁₋₆alkyl, —C(═O)R^{b}, —C(═O)OR^{a}, or C(═O)NR^{a}R^{b};
R^{3b} is hydrogen or C₁₋₂alkyl; and
R⁴ is hydrogen.

7. The compound of claim 2 wherein:
R^{3a} is hydrogen, C₁₋₆alkyl, —C(═O)R^{b}, —C(═O)OR^{a}, or —C(═O)NR^{a}R^{a};
R^{3b} is hydrogen or C₁₋₂alkyl;
R⁴ is hydrogen.
R¹ is hydrogen, —OR^{a}, R^{g}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylOR^{a}, and C₁₋₆alkyl being substituted by 0, 1, 2, or 3 groups selected from fluoro or —OR^{a};
R^{1a} and R^{1b} are hydrogen; and
R^{1c} are independently selected from hydrogen, methyl, or methoxy.

8. The compound of claim 3 wherein:
R^{3a} is hydrogen, C₁₋₆alkyl, —C(═O)R^{b}, —C(═O)OR^{a}, or —C(═O)NR^{a}R^{a};
R^{3b} is hydrogen or C₁₋₂alkyl;
R⁴ is hydrogen.
R¹ is hydrogen, —OR^{a}, R^{g}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylOR^{a}, and C₁₋₆alkyl being substituted by 0, 1, 2, or 3 groups selected from fluoro or —OR^{a};
R^{1a} and R^{1b} are hydrogen; and
R^{1c} are independently selected from hydrogen, hydroxyl, methyl, or methoxy.

9. The compound of claim 6 wherein:
R⁵ is: 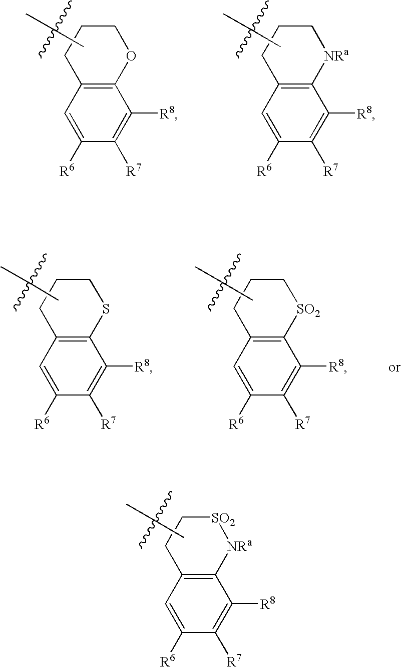
the portion of the each ring above that is attached to the nitrogen atom in Formula (I) is substituted by 0 or 1 substituents selected from R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1, 2 or 3 substituents independently selected from Br, Cl, F and I;
and R⁶, R⁷ and R⁸ are as defined above.

10. The compound of claim 9 wherein:
R² is an unsaturated 5-, 6- or 7-membered monocyclic ring containing 1, 2 or 3 atoms selected from N, O and S, wherein the carbon atoms of the ring are substituted by 0, 1 or 2 oxo groups and the ring is substituted by 0, 1, 2 or 3 substituents selected from R^{e}, R^{g}, C₁₋₄haloalkyl, halo, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and the ring is additionally substituted by 0, 1, 2, 3, 4 or 5 substituents independently selected from Br, Cl, F and I.

11. The compound of claim 8 wherein:
R⁵ is: 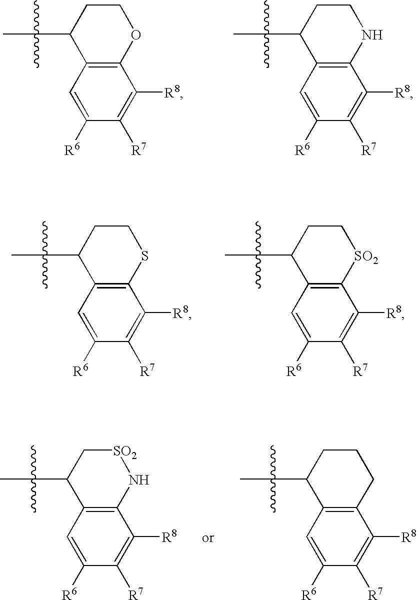
where the portion of the above rings that is attached to the nitrogen atom in Formula (I) is substituted by 0 or 1 substituents selected from R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1, 2 or 3 substituents independently selected from Br, Cl, F and I and R⁶, R⁷ and R⁸ are independently selected from H, a basic moiety, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}, Br, Cl, F and I; provided that 1 or 2 of R⁶, R⁷ and R⁸ are a basic moiety.

12. The compound of claim 11 wherein R² is phenyl substituted by 0, 1, 2 or 3 substituents independently selected from C₁₋₄haloalkyl, halo, C₁₋₆alkyl, or —OR^{a} or naphthyl.

13. The compound of claim 12 wherein R⁶ and R⁸ are H; and R⁷ is selected from amino, aminomethyl, isopropylaminomethyl, t-butylaminomethyl, 2-t-butylaminoethyl, 2-tert-butylamino-1-methyl-ethyl, 1-tert-butylaminoethyl, 1-(tert-butylamino-methyl)-vinyl, 1-(piperidin-1-ylmethyl)-vinyl, N-isobutyl-aminomethyl, N-isobutyl-aminoethyl, (2,2-dimethyl)propylaminomethyl, N-isopropyl-N-ethylaminomethyl, N-isopropyl-N-methylaminomethyl, N-t-butyl-N-methylaminomethyl, N-iso-butyl-N-methylaminomethyl, N-t-butyl-N-ethylaminomethyl, N-isobutyl-N-methylaminomethyl, N-t-butyl-N-isopropylaminomethyl, N,N-di(isopropyl)aminomethyl, N,N-dimethylaminomethyl, N,N-diethylaminomethyl, N,N-di(t-butyl)-aminomethyl, cyclopropylaminomethyl, cyclopropylaminoethyl, cyclopropylmethylaminomethyl, cyclopropylmethylaminoethyl, cyclobutylaminomethyl, cyclobutylaminoethyl, cyclobutylmethylaminomethyl, cyclobutylmethylaminoethyl, 4,5-dihydro-imidazolyl, 1-piperidinylmethyl, 4-fluoropiperidin-1-ylmethyl, 4,4-difluoropiperidin-1-ylmethyl, 3-hydroxypiperidin-1-ylmethyl, 4-hydroxypiperidin-1-ylmethyl, 4-(piperidin-1-yl)piperidinylmethyl, 4-(dimethylamino)piperidin-1-ylmethyl, 2,6-dimethylpiperidin-1-ylmethyl, 4-morpholinylmethyl, 1-pyrrolidinylmethyl, 2-methylpyrrolidin-1-ylmethyl, 2,5-dimethylpyrrolidin-1-ylmethyl, piperazin-1-ylmethyl, azocan-1-ylmethyl, azepan-1-ylmethyl, (7-azabicyclomethyl, (1,3,3-trimethyl-6-azaicyclo[3.2.1]oct-6-yl)methyl, 2-piperidinyl and 4-methylpiperazin-1-ylmethyl.

14. The compound of claim 3 wherein:
R⁴ is hydrogen; and
R⁵ is: 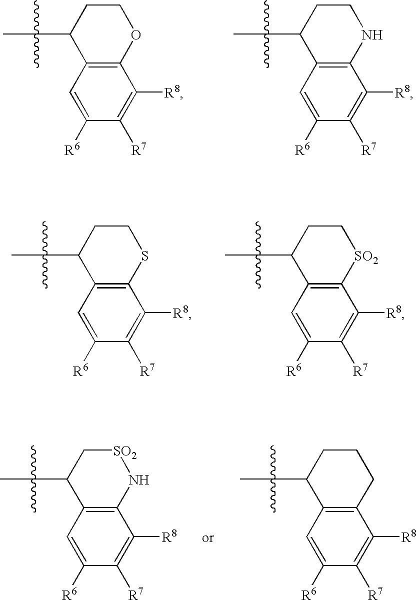
where the portion of the above rings that is attached to the nitrogen atom in Formula (I) is substituted by 0 or 1 substituents selected from R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkylNR^{a}R^{a} and —NR^{a}C₂₋₆alkylOR^{a}, and additionally substituted by 0, 1, 2 or 3 substituents independently selected from Br, Cl, F and I and R⁶, R⁷ and R⁸ are independently selected from H, a basic moiety, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}, Br, Cl, F and I; provided that 1 or 2 of R⁶, R⁷ and R⁸ are a basic moiety.

15. The compound of claim 14 wherein:
R⁶ and R⁸ are H; and R⁷ is selected from amino, aminomethyl, isopropylaminomethyl, t-butylaminomethyl, 2-t-butylaminoethyl, 2-tert-butylamino-1-methyl-ethyl, 1-tert-butylaminoethyl, 1-(tert-butylamino-methyl)-vinyl, 1-(piperidin-1-ylmethyl)-vinyl, N-isobutyl-aminomethyl, N-isobutyl-aminoethyl, (2,2-dimethyl)propylaminomethyl, N-isopropyl-N-ethylaminomethyl, N-isopropyl-N-methylaminomethyl, N-t-butyl-N-methylaminomethyl, N-iso-butyl-N-methylaminomethyl, N-t-butyl-N-ethylaminomethyl, N-isobutyl-N-methylaminomethyl, N-t-butyl-N-isopropylaminomethyl, N,N-di(isopropyl)aminomethyl, N,N-dimethylaminomethyl, N,N-diethylaminomethyl, N,N-di(t-butyl)-aminomethyl, cyclopropylaminomethyl, cyclopropylaminoethyl, cyclopropylmethylaminomethyl, cyclopropylmethylaminoethyl, cyclobutylaminomethyl, cyclobutylaminoethyl, cyclobutylmethylaminomethyl, cyclobutylmethylaminoethyl, 4,5-dihydro-imidazolyl, 1-piperidinylmethyl, 4-fluoropiperidin-1-ylmethyl, 4,4-difluoropiperidin-1-ylmethyl, 3-hydroxypiperidin-1-ylmethyl, 4-hydroxypiperidin-1-ylmethyl, 4-(piperidin-1-yl)-piperidinylmethyl, 4-(dimethylamino)piperidin-1-ylmethyl, 2,6-dimethylpiperidin-1-ylmethyl, 4-morpholinylmethyl, 1-pyrrolidinylmethyl, 2-methylpyrrolidin-1-ylmethyl, 2,5-dimethyl-pyrrolidin-1-ylmethyl, piperazin-1-ylmethyl, azocan-1-ylmethyl, azepan-1-ylmethyl, (7-azabicyclo[2.2.1]hept-7-yl)methyl, (1,3,3-trimethyl-6-azaicyclo[3.2.1]oct-6-yl)methyl, 2-piperidinyl and 4-methylpiperazin-1-ylmethyl.

16. The compound of claim 1 wherein:
R^{3a} is hydrogen, C₁₋₆alkyl, —C(═O)R^{b}, —C(═O)OR^{a}, or —C(═O)NR^{a}R^{a};
R^{3b} is hydrogen or C₁₋₂alkyl;
R⁴ is hydrogen.
R¹ is hydrogen, —OR^{a}, R^{g}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}, and C₁₋₆alkyl being substituted by 0, 1, 2, or 3 groups selected from fluoro or —OR^{a};
R^{1a} and R^{1b} are hydrogen;
R^{1c} are independently selected from hydrogen, hydroxyl, methyl, or methoxy; and
R⁵ is a saturated, partially saturated or unsaturated 8-, 9-, 10- or 11-membered bicyclic or 12-, 13-, 14- or 15-membered tricyclic ring hydrocarbon containing 0, 1, 2, 3 or 4 atoms selected from N, O and S, wherein the carbon and sulfur atoms of the ring are substituted by 0, 1 or 2 oxo groups and the ring is substituted by R⁶, R⁷ or R⁸ independently selected from basic moieties, and additionally substituted by 0, 1, 2 or 3 substituents independently selected from R⁶, R⁷ and R⁸which are independently selected from R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a} and —NR^{a}C₂₋₆alkylOR^{a}, and the ring is additionally substituted by 0, 1, 2, 3, 4 or 5 substituents selected from R⁶, R⁷, R⁸, R⁹ and R¹⁰ which are independently selected from Br, Cl, F and I.

17. The compound of claim 16 wherein:
R⁵ is: 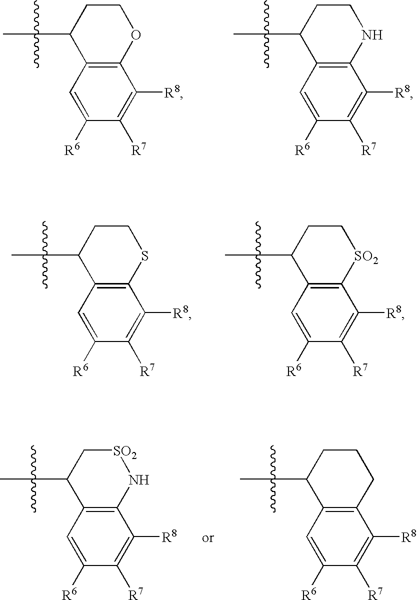
where the portion of the above rings that is attached to the nitrogen atom in Formula (I) is substituted by 0 or 1 substituents selected from R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆ alkylNR^{a}R^{a} and —NR^{a}C₂₋₆alkylOR^{a}, and additionally substituted by 0, 1, 2 or 3 substituents independently selected from Br, Cl, F and I and R⁶, R⁷ and R⁸ are independently selected from H, a basic moiety, R^{g}, C₁₋₈alkyl, C₁₋₄haloalkyl, cyano, nitro, —C(═O)R^{b}, —C(═O)OR^{b}, —C(═O)NR^{a}R^{a}, —C(═NR^{a})NR^{a}R^{a}, —OR^{a}, —OC(═O)R^{b}, —OC(═O)NR^{a}R^{a}, —OC(═O)N(R^{a})S(═O)₂R^{b}, —OC₂₋₆alkylNR^{a}R^{a}, —OC₂₋₆alkylOR^{a}, —SR^{a}, —S(═O)R^{b}, —S(═O)₂R^{b}, —S(═O)₂NR^{a}R^{a}, —S(═O)₂N(R^{a})C(═O)R^{b}, —S(═O)₂N(R^{a})C(═O)OR^{b}, —S(═O)₂N(R^{a})C(═O)NR^{a}R^{a}, —NR^{a}R^{a}, —N(R^{a})C(═O)R^{b}, —N(R^{a})C(═O)OR^{b}, —NR^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkylNR^{a}R^{a}, —NR^{a}C₂₋₆alkylOR^{a}, Br, Cl, F and I; provided that 1 or 2 of R⁶, R⁷ and R⁸ are a basic moiety.

18. The compound of claim 17 wherein:
R² is phenyl substituted by 0, 1, 2 or 3 substituents independently selected from C₁₋₄haloalkyl, halo, C₁₋₄alkyl, or OR^{a} or naphthyl; and
R⁶ and R⁸ are H; and R⁷ is selected from amino, aminomethyl, isopropylaminomethyl, t-butylaminomethyl, 2-t-butylaminoethyl, 2-tert-butylamino-1-methyl-ethyl, 1-tert-butylaminoethyl, 1-(tert-butylamino-methyl)-vinyl, 1-(piperidin-1-ylmethyl)-vinyl, N-isobutyl-aminomethyl, N-isobutyl-aminoethyl, (2,2-dimethyl)propylaminomethyl, N-isopropyl-N-ethylaminomethyl, N-isopropyl-N-methylaminomethyl, N-t-butyl-N-methylaminomethyl, N-iso-butyl-N-methylaminomethyl, N-t-butyl-N-ethylaminomethyl, N-isobutyl-N-methylaminomethyl, N-t-butyl-N-isopropylaminomethyl, N,N-di(isopropyl)aminomethyl, N,N-dimethylaminomethyl, N,N-diethylaminomethyl, N,N-di(t-butyl)-aminomethyl, cyclopropylaminomethyl, cyclopropylaminoethyl, cyclopropylmethylaminomethyl, cyclopropylmethylaminoethyl, cyclobutylaminomethyl, cyclobutylaminoethyl, cyclobutylmethylaminomethyl, cyclobutylmethylaminoethyl, 4,5-dihydro-imidazolyl, 1-piperidinylmethyl, 4-fluoropiperidin-1-ylmethyl, 4,4-difluoropiperidin-1-ylmethyl, 3-hydroxypiperidin-1-ylmethyl, 4-hydroxypiperidin-1-ylmethyl, 4-(piperidin-1-yl)-piperidinylmethyl, 4-(dimethylamino)piperidin-1-ylmethyl, 2,6-dimethylpiperidin-1-ylmethyl, 4-morpholinylmethyl, 1-pyrrolidinylmethyl, 2-methylpyrrolidin-1-ylmethyl, 2,5-dimethyl-pyrrolidin-1-ylmethyl, piperazin-1-ylmethyl, azocan-1-ylmethyl, azepan-1-ylmethyl, (7-azabicyclo[2.2.1]hept-7-yl)methyl, (1,3,3-trimethyl-6-azaicyclo[3.2.1]oct-6-yl)methyl, 2-piperidinyl and 4-methylpiperazin-1-ylmethyl.

19. A compound selected from the group consisting of:
(3R)-4-(2-naphthalenylsulfonyl)-3-phenyl-N-((4R)-7-(1-piperidinylmethyl)-3,4-dihydro-2H-chromen-4-yl)butanamide;
(3S)-3-(acetylamino)-N-((4R)-6-chloro-7-(((1,1-dimethylethyl)amino)methyl)-3,4-dihydro-2H-chromen-4-yl)-4-((3,4-dichlorophenyl)sulfonyl)butanamide;
(3S)-3-(methyloxy)-4-(2-naphthalenylsulfonyl)-N-((1R)-6-(1-piperidinylmethyl)-1,2,3,4-tetrahydro-1-naphthalenyl)butanamide;
(3S)-3-amino-N-((4R)-6-chloro-7-(((1,1-dimethylethyl)amino)methyl)-3,4-dihydro-2H-chromen-4-yl)-4-((3,4-dichlorophenyl)sulfonyl)butanamide;
(3S)-3-hydroxy-4-(2-naphthalenylsulfonyl)-N-((1R)-6-(1-(1-piperidinylmethyl)-ethenyl)-1,2,3,4-tetrahydro-1-naphthalenyl)butanamide;
(3S)-3-hydroxy-4-(2-naphthalenylsulfonyl)-N-((1R)-6-(1-piperidinylmethyl)-1,2,3,4-tetrahydro-1-naphthalenyl)butanamide;
(3S)-3-hydroxy-N-((1R)-6-(((2-methylpropyl)amino)methyl)-1,2,3,4-tetrahydro-1-naphthalenyl)-4-(2-naphthalenylsulfonyl)butanamide;
(3S)-3-hydroxy-N-((1R)-6-((4-methyl-1-piperazinyl)methyl)-1,2,3,4-tetrahydro-1-naphthalenyl)-4-(2-naphthalenylsulfonyl)butanamide;
(3S)-3-hydroxy-N-((1R)-6-(hydroxymethyl)-1,2,3,4-tetrahydro-1-naphthalenyl)-4-(2-naphthalenylsulfonyl)butanamide;
(3S)-3-methyl-4-(2-naphthalenylsulfonyl)-N-((1R)-6-(1-piperidinylmethyl)-1,2,3,4-tetrahydro-1-naphthalenyl)butanamide;
(3S)-3-phenyl-N-((1R)-5-(1-piperidinylmethyl)-2,3-dihydro-1H-inden-1-yl)-4-((3-(trifluoromethyl)phenyl)sulfonyl)butanamide;
(3S)-4-((3,4-dichlorophenyl)sulfonyl)-3-hydroxy-N-((1R)-6-(1-piperidinylmethyl)-1,2,3,4-tetrahydro-1-naphthalenyl)butanamide;
(3S)-4-((4-(1,1-dimethylethyl)phenyl)sulfonyl)-3-hydroxy-N-((1R)-6-(1-piperidinylmethyl)-1,2,3,4-tetrahydro-1-naphthalenyl)butanamide;
(3S)-4-(2-naphthalenylsulfonyl)-3-phenyl-N-((1R)-5-(1-piperidinylmethyl)-2,3-dihydro-1H-inden-1-yl)butanamide;
(3S)-N-((1R)-6-(((1,1-dimethylethyl)amino)methyl)-1,2,3,4-tetrahydro-1-naphthalenyl)-3-phenyl-4-((3-(trifluoromethyl)phenyl)sulfonyl)butanamide;
(3S)-N-((1R)-6-((4-fluoro-1-piperidinyl)methyl)-1,2,3,4-tetrahydro-1-naphthalenyl)-3-hydroxy-4-(2-naphthalenylsulfonyl)butanamide;
(3S)-N-((1R)-6-((cyclopentylamino)methyl)-1,2,3,4-tetrahydro-1-naphthalenyl)-3-hydroxy-4-(2-naphthalenylsulfonyl)butanamide;
(3S)-N-((4R)-6-chloro-7-(((1,1-dimethylethyl)amino)methyl)-3,4-dihydro-2H-chromen-4-yl)-4-((3,4-dichlorophenyl)sulfonyl)-3-hydroxybutanamide;
(R)-2-hydroxy-4-(naphthalen-2-ylsulfonyl)-N-((R)-6-(piperidin-1-ylmethyl)-1,2,3,4-tetrahydronaphthalen-1-yl)butanamide;
1,1-dimethylethyl (1S)-3-(((4R)-6-chloro-7-(((1,1-dimethylethyl)amino)methyl)-3,4-dihydro-2H-chromen-4-yl)amino)-1-(((3,4-dichlorophenyl)sulfonyl)methyl)-3-oxopropylcarbamate;
4-(2-naphthalenylsulfonyl)-N-((1R)-5-(1-piperidinylmethyl)-2,3-dihydro-1H-inden-1-yl)butanamide;
(2R)-2-hydroxy-4-(2-naphthalenyl-sulfonyl)-N-((1R)-6-(1-piperidinylmethyl)-1,2,3,4-tetrahydro-1-naphthalenyl)-butanamide;
(4R,5S)-2,2-dimethyl-5-((naphthalen-2-yl-sulfonyl)methyl)-N-((R)-6-(piperidin-1-ylmethyl)-1,2,3,4-tetrahydronaphthalen-1-yl)-1,3-dioxolane-4-carboxamide;
(2R,3S)-2,3-dihydroxy-4-(naphthalen-2-ylsulfonyl)-N-((R)-6-(piperidin-1-ylmethyl)-1,2,3,4-tetrahydronaphthalen-1-yl)-butanamide;
(S)-4-(naphthalen-2-ylsulfonyl)-3-phenyl-N-((R)-6-(piperidin-1-ylmethyl)-1,2,3,4-tetrahydronaphthalen-1-yl)butanamide;
(R)-4-(naphthalen-2-ylsulfonyl)-3-phenyl-N-((R)-6-(piperidin-1-ylmethyl)-1,2,3,4-tetrahydronaphthalen-1-yl)butanamide;
4-(2-naphthalenylsulfonyl)-N-((1R)-6-(1-piperidinylmethyl)-1,2,3,4-tetrahydro-1-naphthalenyl)butanamide;
(R)-3-(hydroxymethyl)-4-(naphthalen-2-ylsulfonyl)-N-((R)-6-(piperidin-1-ylmethyl)-1,2,3,4-tetrahydronaphthalen-1-yl)-butanamide;
(S)-3-(hydroxymethyl)-4-(naphthalen-2-ylsulfonyl)-N-((R)-6-(piperidin-1-ylmethyl)-1,2,3,4-tetrahydronaphthalen-1-yl)-butanamide;
(R)-4-(naphthalen-2-ylsulfonyl)-3-phenyl-N-(4-(4-(pyridin-4-yl)piperazin-1-yl)-phenyl)butanamide;
(S)-4-(naphthalen-2-ylsulfonyl)-3-phenyl-N-(4-(4-(pyridin-4-yl)piperazin-1-yl)phenyl)-butanamide;
(S)-N-(4-(4,5-dihydro-1H-imidazol-2-yl)phenethyl)-4-(naphthalen-2-ylsulfonyl)-3-phenylbutanamide;
(R)-N-(4-(4,5-dihydro-1H-imidazol-2-yl)-phenethyl)-4-(naphthalen-2-ylsulfonyl)-3-phenylbutanamide;
(2R,3S)-N-((R)-6-chloro-7-(piperidin-1-yl-methyl)chroman-4-yl)-2,3-dihydroxy-4-(naphthalen-2-ylsulfonyl)butanamide;
(2R,3S)-2,3-dihydroxy-N-((R)-6-((4-methyl-piperidin-1-yl)methyl)-1,2,3,4-tetrahydro-naphthalen-1-yl)-4-(naphthalen-2-ylsulfonyl)butanamide;
(2R,3S)-4-(3-chlorophenylsulfonyl)-2,3-dihydroxy-N-((R)-6-(piperidin-1-ylmethyl)-1,2,3,4-tetrahydronaphthalen-1-yl)-butanamide;
(R)-2-methoxy-4-(naphthalen-2-ylsulfonyl)-N-((R)-6-(piperidin-1-ylmethyl)-1,2,3,4-tetrahydronaphthalen-1-yl)butanamide;
(2R,3S)-4-(3,4-dichlorophenylsulfonyl)-2,3-dihydroxy-N-((R)-6-(piperidin-1-ylmethyl)-1,2,3,4-tetrahydronaphthalen-1-yl)-butanamide;
(R)-2-hydroxy-3,3-dimethyl-4-(naphthalen-2-ylsulfonyl)-N-((R)-6-(piperidin-1-ylmethyl)-1,2,3,4-tetrahydronaphthalen-1-yl)butanamide;
(2R,3S)-2,3-dihydroxy-N-((R)-6-(piperidin-1-ylmethyl)-1,2,3,4-tetrahydronaphthalen-1-yl)-4-tosylbutanamide;
(2R,3S)-2,3-dihydroxy-N-((R)-6-(piperidin-1-ylmethyl)-1,2,3,4-tetrahydronaphthalen-1-yl)-4-tosylbutanamide;
(S)-2-hydroxy-4-(naphthalen-2-ylsulfonyl)-N-((R)-6-(piperidin-1-ylmethyl)-1,2,3,4-tetrahydronaphthalen-1-yl)butanamide;
(2R,3S)-4-(4-tert-butylphenylsulfonyl)-2,3-dihydroxy-N-((R)-6-(piperidin-1-ylmethyl)-1,2,3,4-tetrahydronaphthalen-1-yl)butanamide;
(2S,3S)-2,3-dihydroxy-3-(1-(naphthalen-2-ylsulfonyl)cyclopropyl)-N-((R)-6-(piperidin-1-ylmethyl)-1,2,3,4-tetrahydronaphthalen-1-yl)propanamide;
(2R,3R)-2,3-dihydroxy-3-(1-(naphthalen-2-ylsulfonyl)cyclopropyl)-N-((R)-6-(piperidin-1-ylmethyl)-1,2,3,4-tetrahydronaphthalen-1-yl)propanamide;
(2R,3S)-N-((R)-6-((2,6-dimethylpiperidin-1-yl)methyl)-1,2,3,4-tetrahydronaphthalen-1-yl)-2,3-dihydroxy-4-(naphthalen-2-yl-sulfonyl)butanamide;
(2R,3S)-2,3-dihydroxy-N-((R)-6-(((R)-2-methylpiperidin-1-yl)methyl)-1,2,3,4-tetrahydronaphthalen-1-yl)-4-(naphthalen-2-ylsulfonyl)butanamide;
(2R,3S)-2,3-dihydroxy-N-((R)-6-(((S)-2-methylpiperidin-1-yl)methyl)-1,2,3,4-tetrahydronaphthalen-1-yl)-4-(naphthalen-2-ylsulfonyl)butanamide;
(S)-3-hydroxy-3-(1-(naphthalen-2-yl-sulfonyl)cyclopropyl)-N-((R)-6-(piperidin-1-ylmethyl)-1,2,3,4-tetrahydronaphthalen-1-yl)propanamide;
(R)-3-hydroxy-3-(1-(naphthalen-2-yl-sulfonyl)cyclopropyl)-N-((R)-6-(piperidin-1-ylmethyl)-1,2,3,4-tetrahydronaphthalen-1-yl)propanamide;
(2R,3S)-2-hydroxy-3-methyl-N-((R)-6-(piperidin-1-ylmethyl)-1,2,3,4-tetrahydro-naphthalen-1-yl)-4-(3-(trifluoromethyl)-phenylsulfonyl)butanamide;
(2S,3R)-2,3-dihydroxy-N-((R)-6-(piperidin-1-ylmethyl)-1,2,3,4-tetrahydronaphthalen-1-yl)-4-(3-(trifluoromethyl)phenyl-sulfonyl)butanamide;
(2R,3S)-N-((R)-6-((tert-butylamino)methyl)-1,2,3,4-tetrahydronaphthalen-1-yl)-2,3-dihydroxy-4-(3-(trifluoromethyl)-phenylsulfonyl)butanamide;
(R)-3-(1-(naphthalen-2-ylsulfonyl)-cyclopropyl)-N-(6-(piperidin-1-ylmethyl)-1,2,3,4-tetrahydronaphthalen-1-yl)propanamide;
(2R,3S)-2,3-dihydroxy-N-((R)-6-(piperidin-1-ylmethyl)-1,2,3,4-tetrahydronaphthalen-1-yl)-4-(4-(trifluoromethyl)phenyl-sulfonyl)butanamide;
(2R,3S)-4-(2,3-dichlorophenylsulfonyl)-2,3-dihydroxy-N-((R)-6-(piperidin-1-yl-methyl)-1,2,3,4-tetrahydronaphthalen-1-yl)-butanamide;
(2R,3S)-2,3-dihydroxy-N-((R)-6-((4-methylpiperidin-1-yl)methyl)-1,2,3,4-tetrahydro-naphthalen-1-yl)-4-(3-(trifluoromethyl)phenylsulfonyl)butanamide;
(2R,3S)-2,3-dihydroxy-N-((R)-6-(piperidin-1-ylmethyl)-1,2,3,4-tetrahydronaphthalen-1-yl)-4-(3-(trifluoromethyl)phenyl-sulfonyl)butanamide;
(2R,3S)-2,3-dihydroxy-N-((1R)-6-((1R,1S)-1-(1-piperidinyl)ethyl)-1,2,3,4-tetrahydro-1-naphthalenyl)-4-((3-(trifluoromethyl)-phenyl)-sulfonyl)butanamide;
(2R,3S)-2,3-dihydroxy-4-(phenylsulfonyl)-N-((R)-6-(piperidin-1-ylmethyl)-1,2,3,4-tetrahydronaphthalen-1-yl)butanamide;
(2R,3S)-2,3-dihydroxy-N-((R)-6-((R)-1-(piperidin-1-yl)ethyl)-1,2,3,4-tetrahydro-naphthalen-1-yl)-4-(3-(trifluoromethyl)-phenylsulfonyl)butanamide;
(2R,3S)-2,3-dihydroxy-N-((R)-6-((S)-1-(piperidin-1-yl)ethyl)-1,2,3,4-tetrahydro-naphthalen-1-yl)-4-(3-(trifluoromethyl)-phenylsulfonyl)butanamide;
(2R,3S)-2,3-dihydroxy-N-((R)-6-(piperidin-1-ylmethyl)-1,2,3,4-tetrahydronaphthalen-1-yl)-4-(3-(trifluoromethoxy)phenyl-sulfonyl)butanamide;
(2R,3R)-2,3-dihydroxy-N-((R)-6-(piperidin-1-ylmethyl)-1,2,3,4-tetrahydronaphthalen-1-yl)-4-(3-(trifluoromethyl)phenyl-sulfonyl)butanamide;
(2S,3S)-2,3-dihydroxy-N-((1R)-6-(1-piperidinylmethyl)-1,2,3,4-tetrahydro-1-naphthalenyl)-4-((3-(trifluoromethyl)-phenyl)sulfonyl)butanamide;
(2S,3S)-2,3-dihydroxy-N-((R)-6-(piperidin-1-ylmethyl)-1,2,3,4-tetrahydronaphthalen-1-yl)-4-(3-(trifluoromethyl)phenylsulfonyl)-butanamide;
(R)-methyl 2-(naphthalen-2-ylsulfonyl)-5-oxo-5-((R)-6-(piperidin-1-ylmethyl)-1,2,3,4-tetrahydronaphthalen-1-ylamino)pentanoate;
(S)-methyl 2-(naphthalen-2-ylsulfonyl)-5-oxo-5-((R)-6-(piperidin-1-ylmethyl)-1,2,3,4-tetrahydronaphthalen-1-ylamino)pentanoate;
(S)-2-(naphthalen-2-ylsulfonyl)-5-oxo-5-((R)-6-(piperidin-1-ylmethyl)-1,2,3,4-tetrahydronaphthalen-1-ylamino)pentanoic acid;
(R)-2-(naphthalen-2-ylsulfonyl)-5-oxo-5-((R)-6-(piperidin-1-ylmethyl)-1,2,3,4-tetrahydronaphthalen-1-ylamino)pentanoic acid;
1-(((5R)-5-(((2S,3R)-2,3-dihydroxy-4-((3-(trifluoromethyl)phenyl)sulfonyl)butanoyl)-amino)-5,6,7,8-tetrahydro-2-naphthalenyl)methyl)-1-methylpiperidinium;
(2R,3S)-2,3-dihydroxy-N-((R)-7-(piperidin-1-ylmethyl)chroman-4-yl)-4-(3-(trifluoro-methyl)phenylsulfonyl)-butanamide;
(2R,3S)-N-((R)-6-((R)-1-(tert-butylamino)ethyl)-1,2,3,4-tetrahydronaphthalen-1-yl)-2,3-dihydroxy-4-(3-(trifluoromethyl)phenyl-sulfonyl)butanamide;
(2R,3S)-N-((R)-6-((S)-1-(tert-butyl-amino)ethyl)-1,2,3,4-tetrahydro-naphthalen-1-yl)-2,3-dihydroxy-4-(3-(trifluoromethyl)phenylsulfonyl)butanamide;
(S)-3-hydroxy-N-((R)-6-(piperidin-1-ylmethyl)-1,2,3,4-tetrahydronaphthalen-1-yl)-4-(3-(trifluoromethyl)phenyl-sulfonyl)butanamide;
(4R,5S)-2,2-dimethyl-N-((1R)-6-(1-piperidinylmethyl)-1,2,3,4-tetrahydro-1-naphthalenyl)-5-(((2-(trifluoromethyl)-phenyl)sulfonyl)methyl)-1,3-dioxolane-4-carboxamide;
(2R,3S)-2,3-dihydroxy-N-((R)-6-(piperidin-1-ylmethyl)-1,2,3,4-tetrahydronaphthalen-1-yl)-4-(2-(trifluoromethyl)phenylsulfonyl)-butanamide;
(S)-4-(naphthalen-2-ylsulfonyl)-N-((R)-6-(piperidin-1-ylmethyl)-1,2,3,4-tetrahydro-naphthalen-1-yl)pentanamide;
(R)-4-(naphthalen-2-ylsulfonyl)-N-((R)-6-(piperidin-1-ylmethyl)-1,2,3,4-tetrahydro-naphthalen-1-yl)pentanamide;
(R)-2-hydroxy-N-((R)-6-(piperidin-1-ylmethyl)-1,2,3,4-tetrahydronaphthalen-1-yl)-4-(3-(trifluoromethyl)phenylsulfonyl)-butanamide;
(S)-2-hydroxy-N-((R)-6-(piperidin-1-ylmethyl)-1,2,3,4-tetrahydronaphthalen-1-yl)-4-(3-(trifluoromethyl)phenylsulfonyl)-butanamide;
(R)-4-methyl-4-(naphthalen-2-ylsulfonyl)-N-(6-(piperidin-1-ylmethyl)-1,2,3,4-tetrahydronaphthalen-1-yl)pentanamide;
(R)-4-(2,3-dihydrobenzo[b][1,4]dioxin-6-ylsulfonyl)-2-hydroxy-N-((R)-6-(piperidin-1-ylmethyl)-1,2,3,4-tetrahydronaphthalen-1-yl)butanamide;
3-hydroxy-N-(6-(piperidin-1-ylmethyl)-1,2,3,4-tetrahydronaphthalen-1-yl)-4-(2-(trifluoromethoxy)phenylsulfonyl)butanamide;
3-hydroxy-N-(6-(piperidin-1-ylmethyl)-1,2,3,4-tetrahydronaphthalen-1-yl)-4-(4-(trifluoromethoxy)phenylsulfonyl)butanamide;
(R)-2,2-dimethyl-N-(6-(piperidin-1-ylmethyl)-1,2,3,4-tetrahydronaphthalen-1-yl)-4-(3-(trifluoromethyl)phenylsulfonyl)-butanamide;
(R)-N-(6-(piperidin-1-ylmethyl)-1,2,3,4-tetrahydronaphthalen-1-yl)-4-(3-(trifluoro-methyl)phenylsulfonyl)butanamide;
(S)-3-hydroxy-4-methyl-N-((R)-6-(piperidin-1-ylmethyl)-1,2,3,4-tetrahydro-naphthalen-1-yl)-4-(3-(trifluoromethyl)-phenyl-sulfonyl)pentanamide;
(R)-3-hydroxy-4-methyl-N-((R)-6-(piperidin-1-ylmethyl)-1,2,3,4-tetrahydro-naphthalen-1-yl)-4-(3-(trifluoro-methyl)phenylsulfonyl)pentanamide; or
a pharmaceutically acceptable salt thereof.

20. A method of treating a disease mediated by Bradykinin 1 receptor in a patient comprising administering to the patient a pharmaceutical composition comprising a compound of claim 1 and a pharmaceutically acceptable excipient.

21. The method of claim 20 wherein the disease is inflammation, rheumatoid arthritis, cystitis, post-traumatic and post ischemic cerebral edema, liver cirrhosis, Alzheimer's disease, cardiovascular disease, pain, common cold, allergies, asthma, pancreatitis, burns, virus infection, head injury, multiple trauma, rhinitis, hepatorenal failure, diabetes, metastasis, pancreatitis, neovascularization, corneal haze, glaucoma, ocular pain, ocular hypertension or angio edema.

22. The method of claim 20 wherein the disease is osteroarthritis.

23. A pharmaceutical composition comprising a compound of claim 1 and a pharmaceutically acceptable excipient.
